SEQUENCE LISTING (10 sequences)

SEQ ID NO: 1 - protein, 24 aa, Artificial Sequence
  Synthetic
SPQPGAGHDEGPGSGWAAKGTVRG

SEQ ID NO: 2 - protein, 21 aa, Artificial Sequence
  Synthetic
KPGDQILDWQYGVTQAFPHTE

SEQ ID NO: 3 - protein, 500 aa, Artificial Sequence
  Synthetic
MRGELWLLVLVLREAARALSPQPGAGHDEGPGSGWAAKGTVRGWNRRARESPGHVSEPDRTQLSQDLGGGTLAMDTLPDNRTRVVEDNHSYYVSRLYGPSEPHSRELWVDVAEANRSQVKIHTILSNTHRQASRVVLSFDFPFYGHPLRQITIATGGFIFMGDVIHRMLTATQYVAPLMANFNPGYSDNSTVVYFDNGTVFVVQWDHVYLQGWEDKGSFTFQAALHHDGRIVFAYKEIPMSVPEISSSQHPVKTGLSDAFMILNPSPDVPESRRRSIFEYHRIELDPSKVTSMSAVEFTPLPTCLQHRSCDACMSSDLTFNCSWCHVLQRCSSGFDRYRQEWMDYGCAQEAEGRMCEDFQDEDHDSASPDTSFSPYDGDLTTTSSSLFIDSLTTEDDTKLNPYAGGDGLQNNLSPKTKGTPVHLGTIVGIVLAVLLVAAIILAGIYINGHPTSNAALFFIERRPHHWPAMKFRSHPDHSTYAEVEPSGHEKEGFMEAEQC

SEQ ID NO: 4 - protein, 529 aa, Artificial Sequence
  Synthetic
MARFPKADLAAAGVMLLCHFFTDQFQFADGKPGDQILDWQYGVTQAFPHTEEEVEVDSHAYSHRWKRNLDFLKAVDTNRASVGQDSPEPRSFTDLLLDDGQDNNTQIEEDTDHNYYISRIYGPSDSASRDLWVNIDQMEKDKVKIHGILSNTHRQAARVNLSFDFPFYGHFLREITVATGGFIYTGEVVHRMLTATQYIAPLMANFDPSVSRNSTVRYFDNGTALVVQWDHVHLQDNYNLGSFTFQATLLMDGRIIFGYKEIPVLVTQISSTNHPVKVGLSDAFVVVHRIQQIPNVRRRTIYEYHRVELQMSKITNISAVEMTPLPTCLQFNRCGPCVSSQIGFNCSWCSKLQRCSSGFDRHRQDWVDSGCPEESKEKMCENTEPVETSSRTTTTVGATTTQFRVLTTTRRAVTSQFPTSLPTEDDTKIALHLKDNGASTDDSAAEKKGGTLHAGLIIGILILVLIVATAILVTVYMYHHPTSAASIFFIERRPSRWPAMKFRRGSGHPAYAEVEPVGEKEGFIVSEQC

SEQ ID NO: 5 - DNA, 6230 bp, Artificial Sequence
  Synthetic
gctcccggaggccgcagcctccagctccgctcgcgctctcgccgctcctgccggctcgcccggccccgcgctccgccgtctcctcgccgcccgcccctccgccagccccggggaccgcgcggccgcagcctgagccagggccccctccctcgtcaggaccggggcagcaagcaggccgggggcaggtccgggcacccaccatgcgaggcgagctctggctcctggtgctggtgctcagggaggctgcccgggcgctgagcccccagcccggagcaggtcacgatgagggcccaggctctggatgggctgccaaagggaccgtgcggggctggaaccggagagcccgagagagccctgggcatgtgtcagagccggacaggacccagctgagccaggacctgggtgggggcaccctggccatggacacgctgccagataacaggaccagggtggtggaggacaaccacagctattatgtgtcccgtctctatggccccagcgagccccacagccgggaactgtgggtagatgtggccgaggccaaccggagccaagtgaagatccacacaatactctccaacacccaccggcaggcttcgagagtggtcttgtcctttgatttccctttctacgggcatcctctgcggcagatcaccatagcaactggaggcttcatcttcatgggggacgtgatccatcggatgctcacagctactcagtatgtggcgcccctgatggccaacttcaaccctggctactccgacaactccacagttgtttactttgacaatgggacagtctttgtggttcagtgggaccacgtttatctccaaggctgggaagacaagggcagtttcaccttccaggcagctctgcaccatgacggccgcattgtctttgcctataaagagatccctatgtctgtcccggaaatcagctcctcccagcatcctgtcaaaaccggcctatcggatgccttcatgattctcaatccatccccggatgtgccagaatctcggcgaaggagcatctttgaatatcaccgcatagagctggaccccagcaaggtcaccagcatgtcggccgtggagttcaccccattgccgacctgcctgcagcataggagctgtgacgcctgcatgtcctcagacctgaccttcaactgcagctggtgccatgtcctccagagatgctccagtggctttgaccgctatcgccaggagtggatggactatggctgtgcacaggaggcagagggcaggatgtgcgaggacttccaggatgaggaccacgactcagcctcccctgacacttccttcagcccctatgatggagacctcaccactacctcctcctccctcttcatcgacagcctcaccacagaagatgacaccaagttgaatccctatgcaggaggagacggccttcagaacaacctgtcccccaagacaaagggcactcctgtgcacctgggcaccatcgtgggcatcgtgctggcagtcctcctcgtggcggccatcatcctggctggaatttacatcaatggccaccccacatccaatgctgcgctcttcttcatcgagcgtagacctcaccactggccagccatgaagtttcgcagccaccctgaccattccacctatgcggaggtggagccctcgggccatgagaaggagggcttcatggaggctgagcagtgctgagaacaccaagtctcccctttgaagactttgaggccacagaaaagacagttaaagcaaagaagagaagtgacttttcctggcctctcccagcatgccctgggctgagatgagatggtggtttatggctccagagctgctgctcgcttcgtcagcacaccccgaatattgaagagggggccaaaaaacaaccacatggattttttataggaacaacaacctaatctcatcctgttttgatgcaagggttctcttctgtgtcttgtaaccatgaaacagcagaagaactaacataactaactccatttttgtttaaggggcctttacctattcctgcacctaggctaggataactttagagcactgacataaaacgcaaaaacaggaatcatgccgtttgcaaaactaactctgggattaaaggggaagcatgtaaacagctaactgtttttgttaaagatttataggaatgaggaggtttggctattgtcacatgacagactgttagccaaggacaaagaagttctgcaaacctcccctggacccttgctggtgtccagatgtctgcggttgtcagccccttcctttcccccgacctaaacataaaagacaaggcaaagcccgcataattttaagacggttctttaggacattagtccaccatcttcttggtttgctggctctccgaaataaagtccctttccttgctccaactccttgtctctcaacgtattggctatgacgcagcaagcagaatgaatttggactcagttacaggctgtcaatggtctgctctgtagcagtctcagagcctccccgacccactacctggagatagccagatagccagatgccctgctcctggccacctttaaagcccctgcatatgacacaggttaactaaagtcaagattggggctgctgcattccaggttccctagactcacaagctggtccttggccaggtgcagtggctcacgcctgtaatcccagcactttgggaggctgaggcaggcggatcacctgaagtcagaagtttgagaccagcctggccaacataattaaaatgtctctactaaaaatacaaaaaattagctgggtgtggtgacgcttgcctgtatcccagctactcaggaagctgagacacgagaatcacttgaacctgggaggcagaggttgcagtgagctcagatagtgccactgcactccagcctgggtgacagagcgagactccgtctcaaaaaaaaaaaaagaaagcagatcctcatggctatagagttggcattttagccccagcttctgtagctctgaaagcctaaagaaggtattctctccatctgttaaacacagtatagtggctctcagcccttggggcatgttatcatgggagggaagtcaaataagaggagagaaaagaactcaagggggaaactgcatttttaggctttgctctcttaccttgccctttctactcagaaccaataacttctgcatcaaaacatgttacagcctgcatcaagggctttaccccaacctgcagcccagccttccctgggtgagcttgctatgcgcagccacatttaccatgtggggctccctattctgatggcctgttcggtgccgggtttactcactgccctgttctgatgtcagtgcctgtacatacctccaaaggcaggacttgcctgataaatatttttcctcctctgaactggattttataggcattaaagacaagtcgggtggctagagggctccttgagacatacctagcagggaactgcaggtggattctgttgagaggcaaagcacctgagtggttgggacacaggcagctggcatgggagggactttttttgagacagggtctcactgtgtcgcccagggcaaggatgcccaaagacaccaggttggagaggcacctgccaactacttgctttccctggagcctgcatgtgcctgtggggtggggaggcgtaggggtctacggctgcctgagatgggtgtgcacagtgtgtgaagtacctacctccttgccttgctggactgtcagccagtcgcagggccggccacaagacccatgtctccatctggtcatactccatagctaccaagttaacctgctctaaactttggagaactggatctgtccaataaacgcttatttggccaagcctgatggctcgtgcctgtactcccagcactttgggaggctgaggtgggagggttgcttgagcccaggggtttgagaccagcttgggcaacaacaacaaaaatgccaggtgtggtggggtgcacctgtagtcccagctactagggaggctgagccaggaggatcacttgagcccgggaggttgaggctgcagtggggggtcataatcatgccactgtactccagcctgggtgacagagtgagaccctgtctccgaaaaaaaaaaaaaaaaaaagaacggaaaaagaaatgcttacattgtcagggatcctgtagacaatcattaactctatgagatgcttggttctatttttttgggagactttgtccaagtgttttggcttaagaaatccataggcctctcttggtgacacatctctagtactttttgtcataaacaaacaggccatctgccgccaaatacatccactccccatgccactgacatcctatgggtcagccaggcttgctttgactgaggccgaggcatctggaactttctctgcctgcaggggctagcagcagaggcttcaccgcatcaccaccccttcctccactcctgacattctttcccttcagggatccaaaatggttggccgagctcccagtgggaaaacgtgtgctagagttggggagtgagatgagtggtgctgtccatggaatcaggccacagcaggaactgccccactggccatttgagacacacacaggtggtaaatgctctgctggtgggctgtgcttccctcattcagagagctctgttacagcccactgtgtcctttagaagcttgaaaggaacccaactctttgctgcactgtcctttttcttcctcaaattcagaccctccttccaccggcacccccctactccaccctcagctcttccttgcctggtttatcaagcagagctgaggccccacgtttccaactctgattgtcacttgcatcttcacaaaggataaaccacggagcaactggaaaaccatcagccaagcgttcggatgagtctggttattggtccacccccgaccagattcccttacacttaactcacttctttctttggcaatgaccctcatgacatgtataaatgggtatgactaagaagaggctgtgatctaacatttatttgctgccattttttactctggggagaagcagccccaactcatcactgggaaagaactccccctgcaaaccagctaaatttgataatttaaaccccctgcccctaaaacttctcacagagctggggagttggtggcaactttccaagtcaaggtcttgcttagaaagtccttcactacatggccaggtgcagtggctcacgcctgtagtcccaggtacttgggagcctgaggcaggaggattgcttgagctcaggagttcaaggctgcagagagctatgatcatcccactgcatttgtttaaaaataaatttttaaaatttgtgtgttttatcaggggtctcctgtacagtgtatctgtgtatgtttgtgtgtgtgtttgtatacagccttgtttaatgttttgagcaataagatatgcacacacaggtattttgttgctaaagagattggacaaggttgtagctgtgctcaggcttcagcttggtttgttaaattgagagataaacaatgacaagagctgccagccaaccacactattcaaaaagcaaagtgttcaccactaaagctaaccattcatctggttgcaggcaaggctaaggctctctctcctctagttcctggaacagactcacagattggcatgaagcactgatcaggggctgcactcagactccctggccaagcaaacctacaccagaagagtcagtgtcacagatatgatgcggccaatctctgtctccaaaaacctacctgaacttaatggtagaattcaaagatctggggactgagggcacccagccttctaaaacacaatgtattcatgtgtttagtgtaaactctctgcatggattctcagtgttaataataaaaggaagcattcttttacaactcctgctgtgtgcaaaagaaagtgcaaaggatttggagtggcattccgaagatcaccacacataccttggttctgatggctgctgaactccgacttcttcgctgagacatgactgtgggaacagcctccagctatctgctcatcagaggtgctttcctcaacctcctgcaccacctccaagagaaacagcctaaaaagaaaccccagctgtttacttatattggtctgtaaatccctggaagtaaaccccatgcatttttatctactgtctgaggacatacaataaatctgagaaagtc

SEQ ID NO: 6 - protein, 500 aa, Artificial Sequence; identical to SEQ ID NO: 3
  Synthetic

SEQ ID NO: 7 - DNA, 12275 bp, Artificial Sequence
  Synthetic
agactgctgcagccctaaccttcccagggctcagctctttggagctgcccattcctccggctgcgagaaaggacgcgcgccctgcgtcgggcgaagaaaagaagcaaaacttgtcgggagggtttcgtcatcaacctccttcccgcaaacctaaacctcctgccggggccatccctagacagaggaaagttcctgcagagccgaccagccctagtggatctggggcaggcagcggcgctggctgtggaattagatctgttttgaacccagtggagcgcatcgctggggctcggaagtcaccgtccgcgggcaccgggttggcgctgcccgagtggaaccgacagtttgcgagcctcggctgcaagtggcctctcctccccgcggttgttgttcagtgtcgggtgagggctgcgagtgtggcaagttgcaaagagagcctcagaggtccgaagagcgctgcgctcctactcgcgttcgcttcttcctcttctcggttccctactgtgaaatcgcagcgacatttacaaaggcctccgggtcctaccgagaccgatccgcagcgtttggcccggtcgtgcctattgcatcgggagcccccgagcaccggcgaaggactggcgggtggggtagggaggtggcggcggcggcatggcgaggttcccgaaggccgacctggccgctgcaggagttatgttactttgccacttcttcacggaccagtttcagttcgccgatgggaaacccggagaccaaatccttgattggcagtatggagttactcaggccttccctcacacagaggaggaggtggaagttgattcacacgcgtacagccacaggtggaaaagaaacttggactttctcaaggcggtagacacgaaccgagcaagcgtcggccaagactctcctgagcccagaagcttcacagacctgctgctggatgatgggcaggacaataacactcagatcgaggaggatacagaccacaattactatatatctcgaatatatggtccatctgattctgccagccgggatttatgggtgaacatagaccaaatggaaaaagataaagtgaagattcatggaatattgtccaatactcatcggcaagctgcaagagtgaatctgtccttcgattttccattttatggccacttcctacgtgaaatcactgtggcaaccgggggtttcatatacactggagaagtcgtacatcgaatgctaacagccacacagtacatagcacctttaatggcaaatttcgatcccagtgtatccagaaattcaactgtcagatattttgataatggcacagcacttgtggtccagtgggaccatgtacatctccaggataattataacctgggaagcttcacattccaggcaaccctgctcatggatggacgaatcatctttggatacaaagaaattcctgtcttggtcacacagataagttcaaccaatcatccagtgaaagtcggactgtccgatgcatttgtcgttgtccacaggatccaacaaattcccaatgttcgaagaagaacaatttatgaataccaccgagtagagctacaaatgtcaaaaattaccaacatttcggctgtggagatgaccccattacccacatgcctccagtttaacagatgtggcccctgtgtatcttctcagattggcttcaactgcagttggtgtagtaaacttcaaagatgttccagtggatttgatcgtcatcggcaggactgggtggacagtggatgccctgaagagtcaaaagagaagatgtgtgagaatacagaaccagtggaaacttcttctcgaaccaccacaaccgtaggagcgacaaccacccagttcagggtcctaactaccaccagaagagcagtgacttctcagtttcccaccagcctccctacagaagatgataccaagatagcactacatctaaaagataatggagcttctacagatgacagtgcagctgagaagaaagggggaaccctccacgctggcctcatcattggaatcctcatcctggtcctcattgtagccacagccattcttgtgacagtctatatgtatcaccacccaacatcagcagccagcatcttctttattgagagacgcccaagcagatggcctgcgatgaagtttagaagaggctctggacatcctgcctatgctgaagttgaaccagttggagagaaagaaggctttattgtatcagagcagtgctaaaatttctaggacagaacaacaccagtactggtttacaggtgttaagactaaaattttgcctatacctttaagacaaacaaacaaacacacacacaaacaagctctaagctgctgtagcctgaagaagacaagatttctggacaagctcagcccaggaaacaaagggtaaacaaaaaactaaaacttatacaagataccatttacactgaacatagaattccctagtggaatgtcatctatagttcactcggaacatctcccgtggacttatctgaagtatgacaagattataatgcttttggcttaggtgcagggttgcaaagggatcagaaaaaaaaaatcataataaagctttagttcatgagggatcgacacctttggttcaaatgttctctgatgtctcaaagataactgttttccaaagcctgaaccctttcactcaaaagagcaatgatgaatgtctcaagattgctaagaaaaacagcccatgcaagagtgagaacaaacacaaaataagagattttctacattttcaaaacagatgtgtggcaaaaggatgttgtttttctggtctagatccatctgtaccaacaagttcatcactttacagaacgaatctttttatccgtacaggaggttcaaaccatgtctgcctcttcctttgtaatgaatgacctttctatgagctgtgacaaaatttccgaacaattaactaaggatttgggaagagggggtggcaaacggggctttctgttttcctgcctcagcatgaaaacatctgatttatgctttatggaagccttacctccaatccccaactgttaagtcccatgaaaccacagttgctctgggctgatggaaacaaaaggaaacagtatgaagagttccttaatcatttttgaaacaaaaatgttaagggattttaaacatatgattatttttaattttatgccttttcagtactaaacacccatttcattgctgattcctgtctaagaagccattcacgtcagcatggcgatagaaagaatgaaaaaaccctgctgaatcatacagtaattttctttaaagcacatagtagttacataaatatatatatataaatatatttttgtttataactaacacaaggcaggatcttgtgactctaagagtgcgttttgtcatcaaggcaaaacagatgcaagatgcatcactgcattacttccatagagttgtaaaataatccttaatattagaatatttttctgtcacttagcaaaagtggttcagttcattgccgcgcccatcatgttcttgactatttgatccactttttcgtttatgtcaaccccttccctctctggctaaataaagtggatgcagaaagctccttaaatggagatatcgattgccttggaatcacaatcctgattttgaaaattcctcatgaatgaagaaaggaatggcatcccttgagaaggaaagtggttaatatacatactgagctcctaaagtttaaattcaggtactgagtgtacaatttcaccaacattctaacccatgaaacttttacactctgtgccaagaaactgttggcttttgtaaggtacagtgctcaacatttgcagattcaggtctcaagaagcagagatgtctcataagcagcattttcccaacagtttagcatctgtacacatctgccttggtcatcagtccactcacagagtaccatactttatcatcacaagtgtctgacgtgaacgaatgccattttctattccatatattttgctttacaattttaagtatttgatgaagatggtaactttttcctaacttagttaactattaaaaaaaattttgaaaagcaaggtgattgaaggattgtgatgacaatctctttgcagcagctatgtatggtttatgtgaagtatccccacttattcttgtggagcaggtttggtgagacagcaataaccaaatgacatgccaatattactggtgcaactggtattctacaaatgcataaggaacacatagacgacttccttttaggataaaatgatgcttctttcactaccttttgtggtagctgtggcttccaatagcaactgtttgacagttatataaatcttgcatgtgtattcttagtttgtgtcccttaagtactacttaattctcaagtagtaatgttattcttatacccttcagcgttctattttgattcaaaacaattgattcaaaacaattttgaatcaattttctattttgattcaaaacaattgataattctgtaaaaaaacataaacactgaattcttcagtgaaccaaagcaacaaataatagagaaaacttcttgaaactggagtgtgggaaaacttcttaacagaactaagagttaaaggtagtgagaagtgtgtggtgtgtgaattctttagtggtaaggggaaatgtgggctaaatccttttctttcatgaaactctcattctattttatatttctggtttgttcttgttcccatgtgagaaaacatacagtttctgaaaattcaaaatggtcatcaatgctttggactttacataattatgtattagagaaggtgcaactgtacattacttaatatactatgaacataatagaataacaaaaaaagataacaaagagatgcaaacttctggataaatcagataaatggtgctacagaggaatttagttatttcagcttaattatttgtaaaaataataatcagtgactaggtaaagatactgaaactcaagaaaaatatcttgacccatttatgtctagtgttccattattggaacgctaagcttatgggagttatttagagcctactgctcaaggtcatcaccaaggtctggttgcaaaaattcaaaaaattgcaacctcaggcataaatggcttaagcccaagagatggcactgaccataaatggtggcttgtgtgtgaagggtttggacttcaagatatactaacacctagaaaactaaaagtgcaattgacccccaattactattttagtttgaggtatcaagggcatttgccaatctcttatatgtagcttctagagttgtgctgtccaatattatatccactagccaacgtggcttttgaaattaaaattaattaaaattaaattaaaaatgtaattcttcagtcacaccagccacatttcaaaggctcaatagccacgcctggcttgtggctaccatattggataacaagaaacacttccgtggttgaagaaaattttaatggacaatgctgtgtttcatttgaattcgttgtccttgaagaaaagggaagaagaggaaagaaacaagatttggtagctcttgggcatatccaagcatttttgattttagagaggccctatggagtaatgaaaaaaatacaagctgacacagctttaaatccccactgtgttatcttgggaaaatcacttcaacctccttgagtctcaatttgcaattttggaaaagaaaaacaatttttgtcctgtctttcttgcaagattattatgcaacctgattaaatgtacagatgtgcagactcttaaaaaccttatgttaatgtaacccatataaagtacgacttttggtcgagtaccagcacaaatttgcattcaaaggaagaatagttcatcagtgcaaaaagcgttcaaaggtaatcagttcatcacgtatcttgagaaaaagagaatgcattcaaaacacaaagcaaaattgcttcagtttcttttctgcccatctgaatatttcctttagtatttgtcccacatgacatccatcccatgtatttatccactctttcacaacattgcctatgaacatccttcaactggctcagttctttattgcatagagccacttagcttctctagtttcttatgatttcctattttacacttaacagcaaaaggaggcttccatttagaaggcactatctcaaacataaataatttcatatttattttatcactgtaacactaaggagctatggcattaaaatataaaactatttggaacttaatgtaaacctttgcacatgcagactatataaagggaataaaattattttaaattatgctaatatccagatacatattctaaggttagtatcatttatcagtttttcgcaacagattcttatgttacatcttcatgagggagtataaatttgatcacgtccctagtgtctattgcccacttgagaaaggttttgccaggatccacagatcgcttcaagatgctttcgtttatgataggaaattaatatttaaataagactaattgaaaactagattatctggttctaatttcttcctcctgtgaaatgttcttttggtttgtatttctttaggtattttccaagttactgctgcttttacttcacgacctccaaccccacttttgttatcagcgtcatatgccagagtcatggcagatccagcctggacaacatagtgagacccatctctatcaaatatacatatatatatatatatatacacacacacacacacacacacaaatatacatatatatcaaatatatatgtatatttgatagagatgatagcaattatgtgtgtgtgtgtgtgtgtgtgtgtgtgtgtgttagccaagtgtggtttctcatgcctgtagtcccagctactttggaggctgaagtgtaagagttgcttgagcccatgagttagaggctgctgtgagctatgatcatgccactgcactccagcctgggcaacagagcaaaaccccatctctaaataaaaaaaaagaatcgtgagacatcatgtaatccaacctcttaatgttacagataagtaaactgaggcccaaagagatatagtaatagtcccagtgtcagtaaactgggtcttgaacttaaatgtttgtttcagttctcattgccttcatctcttaattgtcggcacttaaggccaaccaagcagcttagacagcgcagtccactcaacggtaactttatttgctatattgacaatctgctctgttctgtaatatagaacattggatcaatctttatctttttgctgacttaaggaggctgctattgttgcaatatccagagatttcaagttctagccattgcggggtctttgtggtgttttcaatttctcctctctccacccctgcccccatctgtggccatctaagttagcaatacaatttattttggccatttaaaatttttctgcctttgttttatttcttcccaaatatatttgaaaatgaatgatctatatagctgattttctgaccacatatataattgcaatttttatttgctcttcaaactattacaacatttttgtccttataaggttttttgggcagataatgcgaggtctgtggtgttaccttttatatactcctattaccttatgtttgtgaagcattttacattttacagagcactttgattcatctccctatttgttcatttatttaactgttcagttcactgaaatatttattgaacacctactggaaccaagtgactgtgctagtaacttgtgatatacagtagtgagagtccaggcatgtaaccgatcattataatacaataacatccctaaaccagactttacaagtagataggatcaacaatactctatcaattttcaactccaattttctattctacttaaaatgggaactagaaactgtttgcctgaagaatgtgtctgaaacataatatatcactgcatgtctgtgctgtagacctgttaattttatctgtgagaaaaaaagttactcaaaattctccctgacctaagaatactttctagttcactgcagtcttactgaccagatgcaacagttgaagtttgatttctcgacccaatatttctattgggttttgaattattaatatcactgttttgaggtattcagaaacaccagtgtatcaaaaaagcatttgcactttaggtgtgtgtggtggttatgtcatttattagaccatcccagacataagacaatcagggaaatcagaaaactccagcctcaaatgtgtctataatttcctgttctaccattgtcatatcataacatggtattacttcttaaggttttgattaagttgatctagcctcaacttaaattgtaatacatctgcctaattattgtctggaataacttttcaaccatccaatgcccactgctctcacaatgattattgtagagaagtaaaatggtaattattcaagtaaagtcacataatttctggagtcagtttttcataacaagtttatggaatacatcatcattggcttcttcataatatatttattatgagtgaccaaattttgcctggaggagcaaaatgctcaaaacttgttattataggttaatttccagctcactgttgacactgaaagattctgtgttactttaaacccaggataaaaggctggaaaaaaaattaaatgtaagtcataaactagtactcagcttttcctagtttctaaggcttattaacatttgcaaattactcaataaatgtctttcataatggaataacataaaagcttttgatttggcagatagtgatattttatttattttcattctggttgaaaaaaatctcagtggcttctcttcattccacgagaattttgatttttaacagcagtctctctttttctcagcattgcaaatatatatgtatatatacattcatgaccaaagtatcgcttactgaccatgcagctgtaaaccttctgtgcctatcaaacaaatacatagcatgaaactaattttagaagtttcatgggggaattttaggggaaagtataaacctaagagtgagtgaatggagatgattcatggaaaaaaaaataaaaatctaaatgtgctattaggcagagttattaacttcttttagttgttgtttgagatggggttttgctcttgttacccaggctggagtgcaatggcgtgatctcgtctcactgcaacctccgcctcccaggttcaagcgattctcctgcctcagccgcccaagtagctgggattacaggcatatgccactacagccggctaattttgtatttttagtagagacggggtttctccatgctggtcaggctggtccccagctcccgacctcaggtgatccgcccacctccgcctcccaaagtgctggaattacaggcctgagccaccgtgcctggccgagttattaacttcttaagagcaatgtgctaataaatactcattgatgaccagctcaaatttaggtcattcagacatccagacactggggcacatattctgcaagcaatgctgagacccctgacatagagaaagcaaaggatatgcctatgattagtctaaaatgcagccatcaccccccatacctcttctatggcattcatcctaacatcatggaggcctttgtcctagagaattatgtgacttgccctagagaattagtgaatgaccaaaaagagactttccatttatcttcctttgacttaaaaggcatgaaaataaggcaaaaaaatcaataaaataattttcctgaggaaaggttaagagatggcactttcctttccgggcccagagctggatttctctgagatttgaccatctcaggactcagacagactccagtgctattttctggacatttgtggtgaagcctcagccaccatgttcaaggtggtttgaatgaaaacatactcagattgtcacatttatagcagggaaataaaaactcaaattaggcatcctgcagcaggctactctgaaaataacattaggcaacatcagagcttcacttgcaaagaaatgttaaaatcattttagggaaatcagtgaagtctctttagaaacagacatcttgtgtatggcgtaacccagtcttggggccctcacggagaagagggggaagtcttttcattgattggtcaaaacaaatctctcatttgcctgatttgaagcatttcaacagtgcctggagagtacattattttcaggggaaaaaaaggagagagtcttttttatatgccagctgggatcatgggaacttcgaatgccaggaatttactactgtttctggtaattctgtgtgtagtcatttgaaatgttgaagtgtgaaaagagaagaaattgggcacttcttgcggcgggggagggggggggggcggtggctttccagattttatgccagttgcaccagcatgcagaatatttgtaatgcatttcaaagtggatataatggcaccctttgtcagaatcacaaagctcactgcggcactgctacaagaggacactgaggaaaatctggccctatgaacctagtcaaccccaagcaaaaagaatgactatgtgtgtgagtgcagcacatggccagttcgtttctcactgttttggaaagccctgtgtgccaaaccaaggacgtgtctttcagggaaaggttaattttccgaagtttattaaaatagaacttggaaaaccaagcattttgaatttattccagtcctctgggcatcattcctatttcttctgccatgtcaaggagaaattccaagcctgcattctgtcatgctaaaataaccagcccatacttctcggtgaccttctgttgaacgtacctgagcctgcaaatgtaaaaatgattgtatctgaatctgcactaatggtgtctgagagcaaaaagagtgtgacctctattggaaacctttgttcaaattcaataattcagagatgctacatacttctgcaagcttcctgattatgttcactgtaatattaatgacctaagtttgaatgtatttccttacagtccattaatttgacatccatcttttacctggggattattacaattgcaataagtcattaatgttttcttcacacagcttcttaaaccaagtttctctgcagctctttcggttctgcttacagtgtgtgggaaatctgatttttttcccctagtaatagtttgataagaaatttagtgtattgactgcctcagtgacacaatttatctttaaaggtgtggaagctggtggggaccaaatgttacctgtgtttttgctgttgattgctattttcagaagcaaaccatgtttttcacttacagtaggagtcaacaaatttgggattttagaagggggaggagggagctatttgtgtaagactgctgtcatatttgactacatattaaaaacagtaaatgagcattttgttttaatttcttaaataccttgtctttcaacatacgttttgtttcctttcttccattagtgttcaaaaggttctacccattgtggaagaaattctgtgtgcagaattcagaggcacaaggctgatggcaagatagaaagttattttgcttctaaacccaccccgatgtggaaactgatactagctagagggagctgtagaaaacaaagatttcaggattgcacagtgtgtgggcaatgggatggagactttttcccctattcccagccacagtgcccaagcgttcaagtctcctggatcagacagatgggattttagctgctgctttaaatcctagtgctggaataagtcaaggtacttcagttcagctcttgcctctgtcactaatcttgctttatgaactcctttgattttctgaataagttccagaaggttctctattattctgtccttcttccaaactggaaatggctgtatctaattctcaggatattttggatgtgtgcctcaggtaatttatgtggaatgtgtaaagcaagatgtctccaattctgaatattccttccccttttcccaatcctccactcttggactacctttataacaacaccgagtacgcacagacctgaacccatgcccaagaagcacacacaatgactggagctgtcgggaattcctgtcagtggcattccctgagcactggctctgtacaactcaattataattttttaagaatcatacctctgtatagatcttttggactgtactgattaaactttgatattgtggagtaaattcagaagtgcaattttaaa

SEQ ID NO: 8 - protein, 529 aa, Artificial Sequence; identical to SEQ ID NO: 4
  Synthetic

SEQ ID NO: 9 - DNA, 12135 bp, Artificial Sequence
  Synthetic
gccctaaccttcccagggctcagctctttggagctgcccattcctccggctgcgagaaaggacgcgcgccctgcgtcgggcgaagaaaagaagcaaaacttgtcgggagggtttcgtcatcaacctccttcccgcaaacctaaacctcctgccggggccatccctagacagaggaaagttcctgcagagccgaccagccctagtggatctggggcaggcagcggcgctggctgtggaattagatctgttttgaacccagtggagcgcatcgctggggctcggaagtcaccgtccgcgggcaccgggttggcgctgcccgagtggaaccgacagtttgcgagcctcggctgcaagtggcctctcctccccgcggttgttgttcagtgtcgggtgagggctgcgagtgtggcaagttgcaaagagagcctcagaggtccgaagagcgctgcgctcctactcgcgttcgcttcttcctcttctcggttccctactgtgaaatcgcagcgacatttacaaaggcctccgggtcctaccgagaccgatccgcagcgtttggcccggtcgtgcctattgcatcgggagcccccgagcaccggcgaaggactggcgggtggggtagggaggtggcggcggcggcatggcgaggttcccgaaggccgacctggccgctgcaggagttatgttactttgccacttcttcacggaccagtttcagttcgccgatgggaaacccggagaccaaatccttgattggcagtatggagttactcaggccttccctcacacagaggaggaggtggaagttgattcacacgcgtacagccacaggtggaaaagaaacttggactttctcaaggcggtagacacgaaccgagcaagcgtcggccaagactctcctgagcccagaagcttcacagacctgctgctggatgatgggcaggacaataacactcagatcgagagagtgaatctgtccttcgattttccattttatggccacttcctacgtgaaatcactgtggcaaccgggggtttcatatacactggagaagtcgtacatcgaatgctaacagccacacagtacatagcacctttaatggcaaatttcgatcccagtgtatccagaaattcaactgtcagatattttgataatggcacagcacttgtggtccagtgggaccatgtacatctccaggataattataacctgggaagcttcacattccaggcaaccctgctcatggatggacgaatcatctttggatacaaagaaattcctgtcttggtcacacagataagttcaaccaatcatccagtgaaagtcggactgtccgatgcatttgtcgttgtccacaggatccaacaaattcccaatgttcgaagaagaacaatttatgaataccaccgagtagagctacaaatgtcaaaaattaccaacatttcggctgtggagatgaccccattacccacatgcctccagtttaacagatgtggcccctgtgtatcttctcagattggcttcaactgcagttggtgtagtaaacttcaaagatgttccagtggatttgatcgtcatcggcaggactgggtggacagtggatgccctgaagagtcaaaagagaagatgtgtgagaatacagaaccagtggaaacttcttctcgaaccaccacaaccgtaggagcgacaaccacccagttcagggtcctaactaccaccagaagagcagtgacttctcagtttcccaccagcctccctacagaagatgataccaagatagcactacatctaaaagataatggagcttctacagatgacagtgcagctgagaagaaagggggaaccctccacgctggcctcatcattggaatcctcatcctggtcctcattgtagccacagccattcttgtgacagtctatatgtatcaccacccaacatcagcagccagcatcttctttattgagagacgcccaagcagatggcctgcgatgaagtttagaagaggctctggacatcctgcctatgctgaagttgaaccagttggagagaaagaaggctttattgtatcagagcagtgctaaaatttctaggacagaacaacaccagtactggtttacaggtgttaagactaaaattttgcctatacctttaagacaaacaaacaaacacacacacaaacaagctctaagctgctgtagcctgaagaagacaagatttctggacaagctcagcccaggaaacaaagggtaaacaaaaaactaaaacttatacaagataccatttacactgaacatagaattccctagtggaatgtcatctatagttcactcggaacatctcccgtggacttatctgaagtatgacaagattataatgcttttggcttaggtgcagggttgcaaagggatcagaaaaaaaaaatcataataaagctttagttcatgagggatcgacacctttggttcaaatgttctctgatgtctcaaagataactgttttccaaagcctgaaccctttcactcaaaagagcaatgatgaatgtctcaagattgctaagaaaaacagcccatgcaagagtgagaacaaacacaaaataagagattttctacattttcaaaacagatgtgtggcaaaaggatgttgtttttctggtctagatccatctgtaccaacaagttcatcactttacagaacgaatctttttatccgtacaggaggttcaaaccatgtctgcctcttcctttgtaatgaatgacctttctatgagctgtgacaaaatttccgaacaattaactaaggatttgggaagagggggtggcaaacggggctttctgttttcctgcctcagcatgaaaacatctgatttatgctttatggaagccttacctccaatccccaactgttaagtcccatgaaaccacagttgctctgggctgatggaaacaaaaggaaacagtatgaagagttccttaatcatttttgaaacaaaaatgttaagggattttaaacatatgattatttttaattttatgccttttcagtactaaacacccatttcattgctgattcctgtctaagaagccattcacgtcagcatggcgatagaaagaatgaaaaaaccctgctgaatcatacagtaattttctttaaagcacatagtagttacataaatatatatatataaatatatttttgtttataactaacacaaggcaggatcttgtgactctaagagtgcgttttgtcatcaaggcaaaacagatgcaagatgcatcactgcattacttccatagagttgtaaaataatccttaatattagaatatttttctgtcacttagcaaaagtggttcagttcattgccgcgcccatcatgttcttgactatttgatccactttttcgtttatgtcaaccccttccctctctggctaaataaagtggatgcagaaagctccttaaatggagatatcgattgccttggaatcacaatcctgattttgaaaattcctcatgaatgaagaaaggaatggcatcccttgagaaggaaagtggttaatatacatactgagctcctaaagtttaaattcaggtactgagtgtacaatttcaccaacattctaacccatgaaacttttacactctgtgccaagaaactgttggcttttgtaaggtacagtgctcaacatttgcagattcaggtctcaagaagcagagatgtctcataagcagcattttcccaacagtttagcatctgtacacatctgccttggtcatcagtccactcacagagtaccatactttatcatcacaagtgtctgacgtgaacgaatgccattttctattccatatattttgctttacaattttaagtatttgatgaagatggtaactttttcctaacttagttaactattaaaaaaaattttgaaaagcaaggtgattgaaggattgtgatgacaatctctttgcagcagctatgtatggtttatgtgaagtatccccacttattcttgtggagcaggtttggtgagacagcaataaccaaatgacatgccaatattactggtgcaactggtattctacaaatgcataaggaacacatagacgacttccttttaggataaaatgatgcttctttcactaccttttgtggtagctgtggcttccaatagcaactgtttgacagttatataaatcttgcatgtgtattcttagtttgtgtcccttaagtactacttaattctcaagtagtaatgttattcttatacccttcagcgttctattttgattcaaaacaattgattcaaaacaattttgaatcaattttctattttgattcaaaacaattgataattctgtaaaaaaacataaacactgaattcttcagtgaaccaaagcaacaaataatagagaaaacttcttgaaactggagtgtgggaaaacttcttaacagaactaagagttaaaggtagtgagaagtgtgtggtgtgtgaattctttagtggtaaggggaaatgtgggctaaatccttttctttcatgaaactctcattctattttatatttctggtttgttcttgttcccatgtgagaaaacatacagtttctgaaaattcaaaatggtcatcaatgctttggactttacataattatgtattagagaaggtgcaactgtacattacttaatatactatgaacataatagaataacaaaaaaagataacaaagagatgcaaacttctggataaatcagataaatggtgctacagaggaatttagttatttcagcttaattatttgtaaaaataataatcagtgactaggtaaagatactgaaactcaagaaaaatatcttgacccatttatgtctagtgttccattattggaacgctaagcttatgggagttatttagagcctactgctcaaggtcatcaccaaggtctggttgcaaaaattcaaaaaattgcaacctcaggcataaatggcttaagcccaagagatggcactgaccataaatggtggcttgtgtgtgaagggtttggacttcaagatatactaacacctagaaaactaaaagtgcaattgacccccaattactattttagtttgaggtatcaagggcatttgccaatctcttatatgtagcttctagagttgtgctgtccaatattatatccactagccaacgtggcttttgaaattaaaattaattaaaattaaattaaaaatgtaattcttcagtcacaccagccacatttcaaaggctcaatagccacgcctggcttgtggctaccatattggataacaagaaacacttccgtggttgaagaaaattttaatggacaatgctgtgtttcatttgaattcgttgtccttgaagaaaagggaagaagaggaaagaaacaagatttggtagctcttgggcatatccaagcatttttgattttagagaggccctatggagtaatgaaaaaaatacaagctgacacagctttaaatccccactgtgttatcttgggaaaatcacttcaacctccttgagtctcaatttgcaattttggaaaagaaaaacaatttttgtcctgtctttcttgcaagattattatgcaacctgattaaatgtacagatgtgcagactcttaaaaaccttatgttaatgtaacccatataaagtacgacttttggtcgagtaccagcacaaatttgcattcaaaggaagaatagttcatcagtgcaaaaagcgttcaaaggtaatcagttcatcacgtatcttgagaaaaagagaatgcattcaaaacacaaagcaaaattgcttcagtttcttttctgcccatctgaatatttcctttagtatttgtcccacatgacatccatcccatgtatttatccactctttcacaacattgcctatgaacatccttcaactggctcagttctttattgcatagagccacttagcttctctagtttcttatgatttcctattttacacttaacagcaaaaggaggcttccatttagaaggcactatctcaaacataaataatttcatatttattttatcactgtaacactaaggagctatggcattaaaatataaaactatttggaacttaatgtaaacctttgcacatgcagactatataaagggaataaaattattttaaattatgctaatatccagatacatattctaaggttagtatcatttatcagtttttcgcaacagattcttatgttacatcttcatgagggagtataaatttgatcacgtccctagtgtctattgcccacttgagaaaggttttgccaggatccacagatcgcttcaagatgctttcgtttatgataggaaattaatatttaaataagactaattgaaaactagattatctggttctaatttcttcctcctgtgaaatgttcttttggtttgtatttctttaggtattttccaagttactgctgcttttacttcacgacctccaaccccacttttgttatcagcgtcatatgccagagtcatggcagatccagcctggacaacatagtgagacccatctctatcaaatatacatatatatatatatatatacacacacacacacacacacacaaatatacatatatatcaaatatatatgtatatttgatagagatgatagcaattatgtgtgtgtgtgtgtgtgtgtgtgtgtgtgtgttagccaagtgtggtttctcatgcctgtagtcccagctactttggaggctgaagtgtaagagttgcttgagcccatgagttagaggctgctgtgagctatgatcatgccactgcactccagcctgggcaacagagcaaaaccccatctctaaataaaaaaaaagaatcgtgagacatcatgtaatccaacctcttaatgttacagataagtaaactgaggcccaaagagatatagtaatagtcccagtgtcagtaaactgggtcttgaacttaaatgtttgtttcagttctcattgccttcatctcttaattgtcggcacttaaggccaaccaagcagcttagacagcgcagtccactcaacggtaactttatttgctatattgacaatctgctctgttctgtaatatagaacattggatcaatctttatctttttgctgacttaaggaggctgctattgttgcaatatccagagatttcaagttctagccattgcggggtctttgtggtgttttcaatttctcctctctccacccctgcccccatctgtggccatctaagttagcaatacaatttattttggccatttaaaatttttctgcctttgttttatttcttcccaaatatatttgaaaatgaatgatctatatagctgattttctgaccacatatataattgcaatttttatttgctcttcaaactattacaacatttttgtccttataaggttttttgggcagataatgcgaggtctgtggtgttaccttttatatactcctattaccttatgtttgtgaagcattttacattttacagagcactttgattcatctccctatttgttcatttatttaactgttcagttcactgaaatatttattgaacacctactggaaccaagtgactgtgctagtaacttgtgatatacagtagtgagagtccaggcatgtaaccgatcattataatacaataacatccctaaaccagactttacaagtagataggatcaacaatactctatcaattttcaactccaattttctattctacttaaaatgggaactagaaactgtttgcctgaagaatgtgtctgaaacataatatatcactgcatgtctgtgctgtagacctgttaattttatctgtgagaaaaaaagttactcaaaattctccctgacctaagaatactttctagttcactgcagtcttactgaccagatgcaacagttgaagtttgatttctcgacccaatatttctattgggttttgaattattaatatcactgttttgaggtattcagaaacaccagtgtatcaaaaaagcatttgcactttaggtgtgtgtggtggttatgtcatttattagaccatcccagacataagacaatcagggaaatcagaaaactccagcctcaaatgtgtctataatttcctgttctaccattgtcatatcataacatggtattacttcttaaggttttgattaagttgatctagcctcaacttaaattgtaatacatctgcctaattattgtctggaataacttttcaaccatccaatgcccactgctctcacaatgattattgtagagaagtaaaatggtaattattcaagtaaagtcacataatttctggagtcagtttttcataacaagtttatggaatacatcatcattggcttcttcataatatatttattatgagtgaccaaattttgcctggaggagcaaaatgctcaaaacttgttattataggttaatttccagctcactgttgacactgaaagattctgtgttactttaaacccaggataaaaggctggaaaaaaaattaaatgtaagtcataaactagtactcagcttttcctagtttctaaggcttattaacatttgcaaattactcaataaatgtctttcataatggaataacataaaagcttttgatttggcagatagtgatattttatttattttcattctggttgaaaaaaatctcagtggcttctcttcattccacgagaattttgatttttaacagcagtctctctttttctcagcattgcaaatatatatgtatatatacattcatgaccaaagtatcgcttactgaccatgcagctgtaaaccttctgtgcctatcaaacaaatacatagcatgaaactaattttagaagtttcatgggggaattttaggggaaagtataaacctaagagtgagtgaatggagatgattcatggaaaaaaaaataaaaatctaaatgtgctattaggcagagttattaacttcttttagttgttgtttgagatggggttttgctcttgttacccaggctggagtgcaatggcgtgatctcgtctcactgcaacctccgcctcccaggttcaagcgattctcctgcctcagccgcccaagtagctgggattacaggcatatgccactacagccggctaattttgtatttttagtagagacggggtttctccatgctggtcaggctggtccccagctcccgacctcaggtgatccgcccacctccgcctcccaaagtgctggaattacaggcctgagccaccgtgcctggccgagttattaacttcttaagagcaatgtgctaataaatactcattgatgaccagctcaaatttaggtcattcagacatccagacactggggcacatattctgcaagcaatgctgagacccctgacatagagaaagcaaaggatatgcctatgattagtctaaaatgcagccatcaccccccatacctcttctatggcattcatcctaacatcatggaggcctttgtcctagagaattatgtgacttgccctagagaattagtgaatgaccaaaaagagactttccatttatcttcctttgacttaaaaggcatgaaaataaggcaaaaaaatcaataaaataattttcctgaggaaaggttaagagatggcactttcctttccgggcccagagctggatttctctgagatttgaccatctcaggactcagacagactccagtgctattttctggacatttgtggtgaagcctcagccaccatgttcaaggtggtttgaatgaaaacatactcagattgtcacatttatagcagggaaataaaaactcaaattaggcatcctgcagcaggctactctgaaaataacattaggcaacatcagagcttcacttgcaaagaaatgttaaaatcattttagggaaatcagtgaagtctctttagaaacagacatcttgtgtatggcgtaacccagtcttggggccctcacggagaagagggggaagtcttttcattgattggtcaaaacaaatctctcatttgcctgatttgaagcatttcaacagtgcctggagagtacattattttcaggggaaaaaaaggagagagtcttttttatatgccagctgggatcatgggaacttcgaatgccaggaatttactactgtttctggtaattctgtgtgtagtcatttgaaatgttgaagtgtgaaaagagaagaaattgggcacttcttgcggcgggggagggggggggggcggtggctttccagattttatgccagttgcaccagcatgcagaatatttgtaatgcatttcaaagtggatataatggcaccctttgtcagaatcacaaagctcactgcggcactgctacaagaggacactgaggaaaatctggccctatgaacctagtcaaccccaagcaaaaagaatgactatgtgtgtgagtgcagcacatggccagttcgtttctcactgttttggaaagccctgtgtgccaaaccaaggacgtgtctttcagggaaaggttaattttccgaagtttattaaaatagaacttggaaaaccaagcattttgaatttattccagtcctctgggcatcattcctatttcttctgccatgtcaaggagaaattccaagcctgcattctgtcatgctaaaataaccagcccatacttctcggtgaccttctgttgaacgtacctgagcctgcaaatgtaaaaatgattgtatctgaatctgcactaatggtgtctgagagcaaaaagagtgtgacctctattggaaacctttgttcaaattcaataattcagagatgctacatacttctgcaagcttcctgattatgttcactgtaatattaatgacctaagtttgaatgtatttccttacagtccattaatttgacatccatcttttacctggggattattacaattgcaataagtcattaatgttttcttcacacagcttcttaaaccaagtttctctgcagctctttcggttctgcttacagtgtgtgggaaatctgatttttttcccctagtaatagtttgataagaaatttagtgtattgactgcctcagtgacacaatttatctttaaaggtgtggaagctggtggggaccaaatgttacctgtgtttttgctgttgattgctattttcagaagcaaaccatgtttttcacttacagtaggagtcaacaaatttgggattttagaagggggaggagggagctatttgtgtaagactgctgtcatatttgactacatattaaaaacagtaaatgagcattttgttttaatttcttaaataccttgtctttcaacatacgttttgtttcctttcttccattagtgttcaaaaggttctacccattgtggaagaaattctgtgtgcagaattcagaggcacaaggctgatggcaagatagaaagttattttgcttctaaacccaccccgatgtggaaactgatactagctagagggagctgtagaaaacaaagatttcaggattgcacagtgtgtgggcaatgggatggagactttttcccctattcccagccacagtgcccaagcgttcaagtctcctggatcagacagatgggattttagctgctgctttaaatcctagtgctggaataagtcaaggtacttcagttcagctcttgcctctgtcactaatcttgctttatgaactcctttgattttctgaataagttccagaaggttctctattattctgtccttcttccaaactggaaatggctgtatctaattctcaggatattttggatgtgtgcctcaggtaatttatgtggaatgtgtaaagcaagatgtctccaattctgaatattccttccccttttcccaatcctccactcttggactacctttataacaacaccgagtacgcacagacctgaacccatgcccaagaagcacacacaatgactggagctgtcgggaattcctgtcagtggcattccctgagcactggctctgtacaactcaattataattttttaagaatcatacctctgtatagatcttttggactgtactgattaaactttgatattgtggagtaaattcagaagtgcaattttaaaaaaaaaaaaaaaaaaaaa

SEQ ID NO: 10 - protein, 480 aa, Artificial Sequence
  Synthetic
MARFPKADLAAAGVMLLCHFFTDQFQFADGKPGDQILDWQYGVTQAFPHTEEEVEVDSHAYSHRWKRNLDFLKAVDTNRASVGQDSPEPRSFTDLLLDDGQDNNTQIERVNLSFDFPFYGHFLREITVATGGFIYTGEVVHRMLTATQYIAPLMANFDPSVSRNSTVRYFDNGTALVVQWDHVHLQDNYNLGSFTFQATLLMDGRIIFGYKEIPVLVTQISSTNHPVKVGLSDAFVVVHRIQQIPNVRRRTIYEYHRVELQMSKITNISAVEMTPLPTCLQFNRCGPCVSSQIGFNCSWCSKLQRCSSGFDRHRQDWVDSGCPEESKEKMCENTEPVETSSRTTTTVGATTTQFRVLTTTRRAVTSQFPTSLPTEDDTKIALHLKDNGASTDDSAAEKKGGTLHAGLIIGILILVLIVATAILVTVYMYHHPTSAASIFFIERRPSRWPAMKFRRGSGHPAYAEVEPVGEKEGFIVSEQC

(19) United States
(12) Patent Application Publication
(10) Pub. No.: US 2024/0118284 A1
(43) Pub. Date: Apr. 11, 2024

(54) COMPOSITIONS AND METHODS FOR DETECTING PLXDC1 AND PLXCD2 IN HUMAN TISSUES

(71) Applicant: THE REGENTS OF THE UNIVERSITY OF CALIFORNIA, Oakland, CA (US)

(72) Inventors: Hui SUN, Los Angeles, CA (US); Adrian Chichuen AU, Los Angeles, CA (US); Guo Cheng, Los Angeles, CA (US)

(73) Assignee: THE REGENTS OF THE UNIVERSITY OF CALIFORNIA, Oakland, CA (US)

(21) Appl. No.: 17/769,171

(22) PCT Filed: Oct. 16, 2020
(86) PCT No.: PCT/US2020/056003

§ 371 Date: Apr. 14, 2022

Related U.S. Application Data
(60) Provisional application No. 62/923,029, filed on Oct. 18, 2019.

(51) Int. Cl. G01N 33/574; C07K 16/28; G01N 1/30

(52) U.S. Cl. CPC G01N 33/57492; C07K 16/28; G01N 1/30; G01N 33/57438

(57) ABSTRACT

The present disclosure relates generally to methods for preparing a tissue sample for detection of the expression of a transmembrane protein in the tissue sample. The present disclosure also provides antibodies and treatments targeting tumor samples expressing the PLXDC1 or the PLXDC2 proteins.

### CROSS REFERENCE TO RELATED APPLICATIONS

This application claims the benefit under 35 U.S.C. § 119(e) of U.S. Provisional Application Ser. No. 62/923,029 filed Oct. 18, 2019, the content of which is incorporated by reference in its entirety into the present disclosure.

### BACKGROUND

Plexin domain containing 1 (PLXDC1, or TEM7) is a cell-surface transmembrane domain protein that can be a therapeutic target to treat angiogenesis-dependent human diseases. PLXDC1 is highly enriched in pathogenic blood vessels of diverse types of human tumors and of diabetic retinopathy, a major cause of blindness.

Future development of therapies that target PLXDC1 to suppress tumor angiogenesis can be facilitated by the detection of PLXDC1 expression in human cancer patients. A patient whose tumor has high PLXDC1 expression would be most suitable for new therapies that target this therapeutic target. There is a need to develop a highly sensitive method to detect PLXDC1 in human pathology samples for clinical diagnosis and to investigate the expression pattern of PLXDC2, a less known homologue of PLXDC1, in disease samples.

### SUMMARY

The present disclosure, in various embodiments, describes methods for preparing tissue samples for immunohistochemical analysis of the expression of PLXDC1 or PLXDC2 proteins. Antibodies that can be used for such analysis are also described, which can also be used for treating diseases that express these proteins. In accordance with certain embodiments of the disclosure, therefore, provided is a method for preparing a tissue sample for detection of the expression of a transmembrane protein in the tissue sample, comprising: sectioning a tissue slide from the tissue sample, and fixing the tissue slide in a non-crosslinking fixative at a temperature of about 0° C. to 25° C.

In some embodiments, the non-crosslinking fixative is selected from the group consisting of methanol, ethanol, acetone, acetic acid and combinations thereof. In some embodiments, the non-crosslinking fixative is methanol. In some embodiments, the non-crosslinking fixative is used at a concentration that is at least 99%. The non-crosslinking fixative can also be a mixture of agents, such as ethanol and acetic acid at a ratio of about 2:1 to about 4:1 (v/v). In some embodiments, the tissue slide is fixed in the non-crosslinking fixative for about 2 hours to about 24 hours. In some embodiments, the tissue slide is fixed in the non-crosslinking fixative for 5 to 16 hours.

In some embodiments, the method further comprises drying the tissue slide prior to fixing. In some embodiments, the fixing starts within 16 hours following the sectioning. In some embodiments, the fixing starts within 2 hours following the sectioning. In some embodiments, the fixing starts within 30 minutes following the drying.

In some embodiments, the tissue block and tissue slide are not treated with a crosslinking fixative. In some embodiments, the crosslinking fixative comprises paraformaldehyde, formaldehyde, glutaraldehyde acrolein, and osmium tetroxide.

In some embodiments, the transmembrane protein is Plexin domain containing 1 (PLXDC1) or Plexin domain containing 2 (PLXDC2). In some embodiments, the method further comprises detecting the transmembrane protein with immunohistochemical staining of the tissue slide. In some embodiments, the immunohistochemical staining uses an antibody that recognizes at least an amino acid residue of the PLXDC1 protein within SEQ ID NO:1 (SPQPGAGHDEGPGSGWAAKGTVRG). In some embodiments, the immunohistochemical staining uses an antibody that recognizes at least an amino acid residue of the PLXDC2 protein within SEQ ID NO:2 (KPGDQILDWQYGVTQAFPHTE).

In some embodiments, the tissue sample was frozen within two hours after isolation from a human patient. In some embodiments, the tissue sample comprises a blood vessel. In some embodiments, the human patient suffers from tumor or diabetic retinopathy. In some embodiments, the tissue slide has a thickness of about 1 micrometer to about 25 micrometers.

Also provided, in one aspect, is an antibody or antigen-binding fragment thereof having binding specificity to a human Plexin domain containing 1 (PLXDC1) protein, wherein the antibody or fragment thereof is capable of binding to at least one of the amino acid residues within SEQ ID NO:1 (SPQPGAGHDEGPGSGWAAKGTVRG). In some embodiments, the antibody or fragment thereof is capable of binding to at least two non-adjacent amino acid residues within SEQ ID NO:1. In some embodiments, the binding between the antibody or fragment thereof and amino acid residues in SEQ ID NO:1 is sufficient to maintain the binding specificity between the antibody or fragment thereof and the PLXDC1 protein.

In some embodiments, the antibody or fragment thereof is a polyclonal antibody or fragment thereof, or is a monoclonal antibody or fragment thereof.

In some embodiments, the antibody or fragment thereof is obtained by immunizing an animal with a fusion protein comprising a fragment of PLXDC1 shorter than 50 amino acid residues and comprising at least 50% of SEQ ID NO:1. In some embodiments, the fragment comprises SEQ ID NO:1.

Still further, provided is an antibody or antigen-binding fragment thereof having binding specificity to a human Plexin domain containing 2 (PLXDC2) protein, wherein the antibody or fragment thereof is capable of binding to at least one of the amino acid residues within SEQ ID NO:2 (KPGDQILDWQYGVTQAFPHTE). In some embodiments, the antibody or fragment thereof is capable of binding to at least two non-adjacent amino acid residues within SEQ ID NO:2. In some embodiments, the binding between the antibody or fragment thereof and amino acid residues in SEQ ID NO:2 is sufficient to maintain the binding specificity between the antibody or fragment thereof and the PLXDC2 protein.

In some embodiments, the antibody or fragment thereof is obtained by immunizing an animal with a fusion protein comprising a fragment of PLXDC2 shorter than 50 amino acid residues and comprising at least 50% of SEQ ID NO:2. In some embodiments, the fragment comprises SEQ ID NO:1.

A method is provided, in some embodiments, for detecting the expression of PLXDC1 or PLXDC2 in a human sample, comprising contacting the sample with an antibody or fragment of the present disclosure, and detecting the binding of the antibody to the PLXDC1 or PLXDC2 in the sample.

In other embodiments, a method of treating a cancer patient has PLXDC1 or PLXDC2 expressed in a cancer endothelial or tumor cell is provided, comprising administering to the patient an antibody or fragment of the present disclosure.

Still in another aspect, provided is a method for identifying a human cancer patient suitable for an anti-PLXDC2 (Plexin domain containing 2) therapy, comprising detecting the expression of the PLXDC2 protein in a liver cancer tumor sample isolated from the patient, wherein expression of the PLXDC2 protein in the liver sample indicates that the patient is suitable for a therapy comprising an agent that inhibits the PLXDC2 signaling. In some embodiments, the agent is an anti-PLXDC2 antibody.

Yet other embodiments provide a method for treating a human cancer patient identified as having expression of the PLXDC2 (Plexin domain containing 2) protein in the liver, comprising administering to the patient an agent that inhibits the PLXDC2 signaling. In some embodiments, the patient suffers from liver cancer or a metastatic cancer that has spread to liver. In some embodiments, the agent is an anti-PLXDC2 antibody.

### BRIEF DESCRIPTION OF THE DRAWINGS

FIGS. 1A-1D show the binding specificity of polyclonal antibodies obtained for PLXDC1 and PLXDC2 in immunostaining. FIG. 1A shows that polyclonal antibody against human TEM7 recognized human TEM7 transfected into HEK293 cells. FIG. 1B shows that polyclonal antibody against human TEM7 did not recognize human PLXDC2 transfected into HEK293 cells or untransfected cells. FIG. 1C shows that polyclonal antibody against human PLXDC2 did not recognize human TEM7 transfected into HEK293 cells or untransfected cells. FIG. 1D shows that polyclonal antibody against human PLXDC2 recognized human PLXDC2 transfected into HEK293 cells. Antibody staining signal is in green and cell nuclei are in blue. Cells were fixed with 100% methanol.

FIGS. 2A-2D demonstrate the importance of sufficient fixation time in revealing the transmembrane proteins. Five hours of methanol fixation (FIG. 2A) revealed more robust TEM7 signals in tumor blood vessels than two hours of methanol fixation (FIG. 2B) of fresh frozen tumor sections. TEM7 was detected using the polyclonal antibody against TEM7. As a control, the tumor sections were stained using antibody against VEGFR2, a marker of blood vessels (FIG. 2C). A control staining by omitting the primary antibody is shown in FIG. 2D. All sections are from the same human liver cancer tumor (hepatocellular carcinoma). Immunostaining signal is in brown color.

FIGS. 3A-3F show the expression of PLXDC1 and VEGFR2 in human liver cancer samples. FIGS. 3A and 3B show that polyclonal antibody against human TEM7 recognized human TEM7 expressed in tumor blood vessels in human liver cancer. FIGS. 3C and 3D show control immunostaining using antibody against VEGFR2, a known marker of blood vessels in different sections of the same tumor. FIGS. 3E and 3F show control immunostaining without the primary antibody (but with all other steps). Antibody staining signal is in brown color.

FIGS. 4A-4F show the expression of PLXDC1, PLXDC2 and VEGFR2 in the blood vessels of liver cancer samples. FIGS. 4A-4C show staining in the region of the tumor that has abundant large tumor vessels. FIGS. 4D-4F show staining in the region of the tumor that has mostly tumor microvessels. FIGS. 4A and 4D show that polyclonal antibody against human TEM7 recognizes human TEM7 expressed in tumor blood vessels in human liver cancer. FIGS. 4B and 4E show that polyclonal antibody against human PLXDC2 recognized human PLXDC2 expressed in tumor blood vessels in human liver cancer. FIGS. 4C and 4F show control immunostaining using antibody against VEGFR2, a known marker of blood vessels in different sections of the same tumor. Antibody staining signal is in brown color.

FIGS. 5A-5B show immunostaining of a metastatic human tumor from colon cancer with polyclonal antibodies against human PLXDC1 and PLXDC2. FIG. 5A shows that polyclonal antibody against human TEM7 recognized human TEM7 expressed in tumor blood vessels in human metastatic colon cancer. FIG. 5B shows that polyclonal antibody against human PLXDC2 recognized human PLXDC2 expressed in tumor blood vessels in human metastatic colon cancer. Like PLXDC1, PLXDC2 was also highly enriched in tumor blood vessels in this metastatic colon cancer tumor. Antibody staining signal is in brown.

FIGS. 6A-D show immunostaining of human pancreatic tumor tissues with polyclonal antibodies against human PLXDC1 and PLXDC2. FIG. 6A shows polyclonal antibody against human TEM7 recognized human TEM7 expressed in tumor blood vessels of a pancreatic tumor tissue. FIG. 6B shows that as a positive control, an antibody against von Willebrand factor (vWF), a general marker of blood vessels, demonstrates the effectiveness of the fixation method. FIG. 6C shows that unlike TEM7, PLXDC2 was not expressed in these samples. FIG. 6D presents control staining without a primary antibody.

It will be recognized that some or all of the figures are schematic representations for purpose of illustration.

### DETAILED DESCRIPTION

The following description sets forth exemplary embodiments of the present technology. It should be recognized, however, that such description is not intended as a limitation on the scope of the present disclosure but is instead provided as a description of exemplary embodiments.

Plexin domain containing 1 (PLXDC1, or TEM7) is a cell-surface transmembrane domain protein that can be a therapeutic target to treat angiogenesis-dependent human diseases. PLXDC1 is highly enriched in pathogenic blood vessels of diverse types of human tumors and of diabetic retinopathy, a major cause of blindness. The identification of PLXDC1 as the cell-surface receptor for PEDF, an endogenous anti-angiogenic factor is consistent with the specificity of PEDF's inhibitory activity against pathogenic blood vessels and paved the way to develop new therapies based on this ligand/receptor pair.

Based on the observations that PLXDC1 is highly expressed in tumors (e.g., tumor blood vessels), anti-PLXDC1 polyclonal antibodies were generated. The generated polyclonal antibodies have binding specificity to a PLXDC1 protein. For example, the PLXDC1 polyclonal antibodies may be capable of binding to SEQ ID NO:1 (SPQPGAGHDEGPGSGWAAKGTVRG) of PLXDC1. Polyclonal antibodies against PLXDC2, a less known homologue of PLXDC1, were also generated. For example, the PLXDC2 polyclonal antibodies may be capable of binding to SEQ ID NO:2 (KPGDQILDWQYGVTQAFPHTE) of PLXDC2.

PLXDC1, PLXDC2, and especially their disclosed epitopes (i.e., SEQ ID NO 1 and 2, respectively) are difficult to detect using the standard immunostaining methods. Disclosed herein are methods for preparing a tissue sample for detection of the expression of a transmembrane protein (e.g., PLXDC1 or PLXDC2) in the tissue sample.

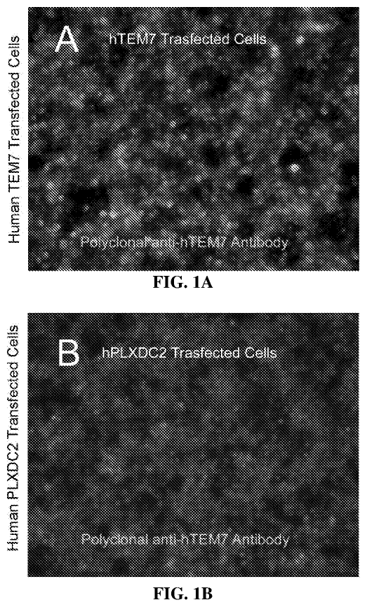

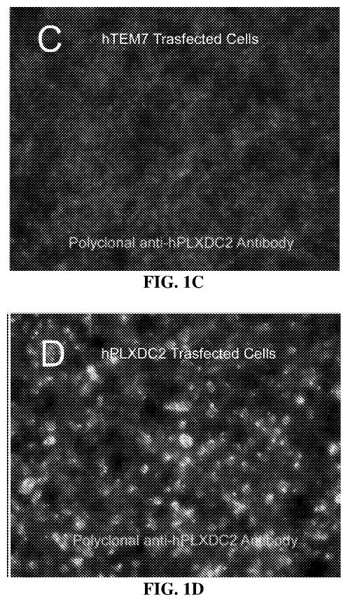

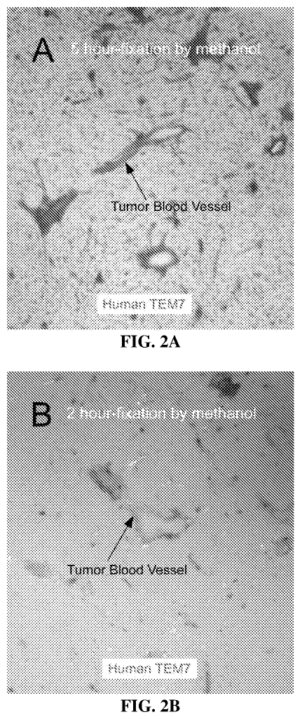

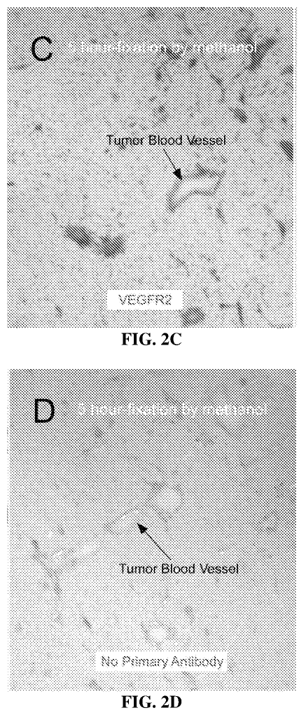

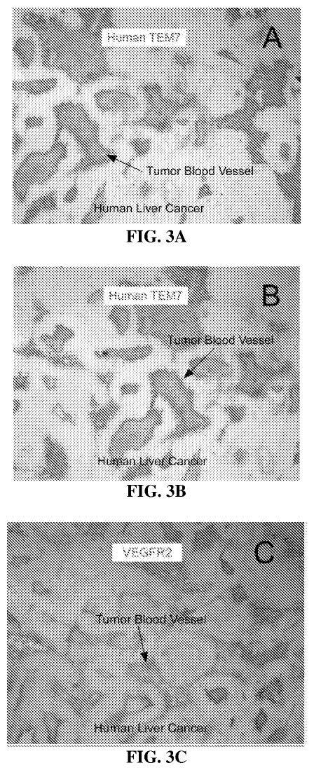

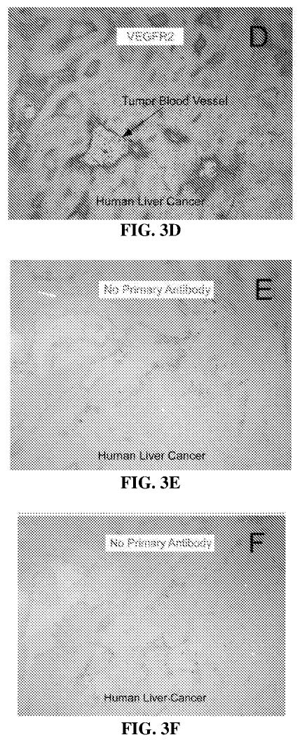

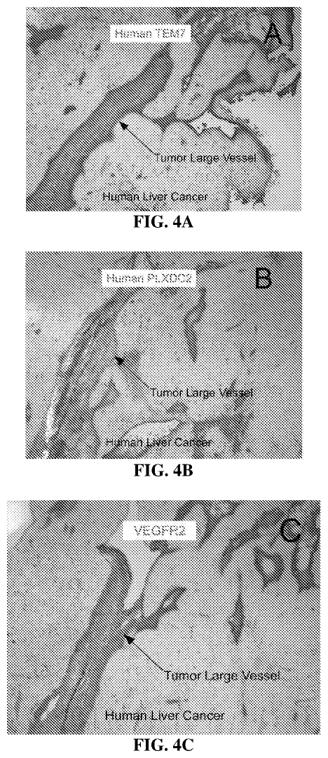

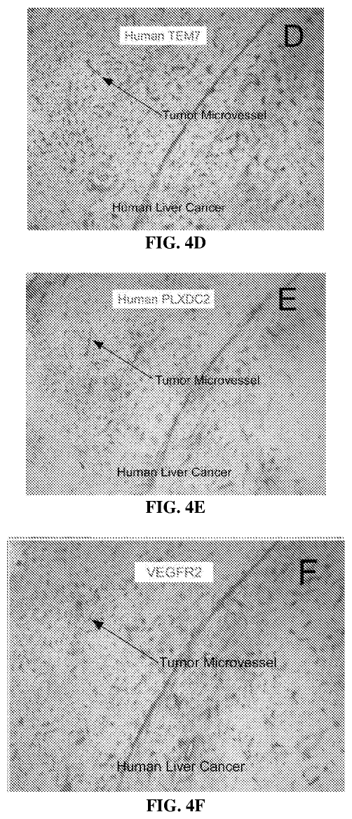

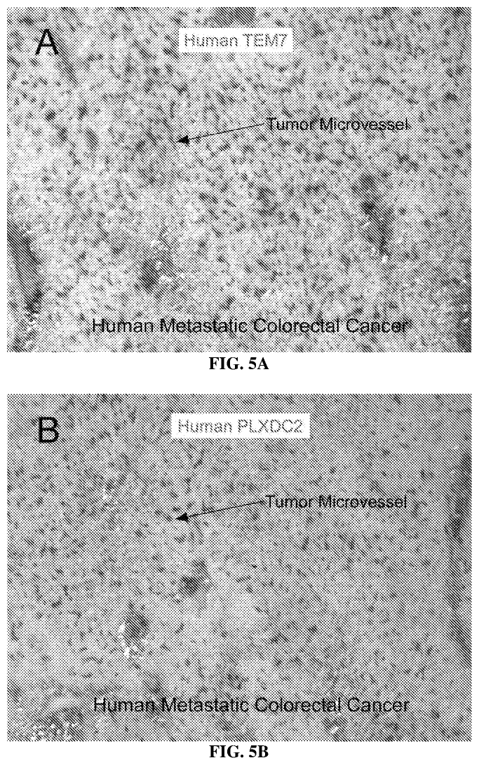

The disclosed polyclonal antibodies specifically recognized human TEM7 (PLXDC1) and PLXDC2 in immunostaining (FIG. 1A-1D). The disclosed immunostaining methods were successful in detecting PLXDC1 and PLXDC2 in the human tumor samples. Particular fixatives (e.g., methanol) and specific fixation times (e.g., five hours) allowed the ability to immunostain PLXDC1 and PLXDC2 in the human tumor samples (FIG. 2A-2D). The disclosed methods and antibodies were used to detect PLXDC1 and PLXDC2 in tumor blood vessels in human liver cancer (FIG. 3A-3F). The disclosed methods and antibodies were also used to differentiate between abundant large tumor vessels human liver cancer tumor (hepatocellular carcinoma) versus tumor microvessels (FIG. 4A-4F). The disclosed methods and antibodies were also used to detect human PLXDC1 and PLXDC2 expressed in tumor blood vessels in human metastatic colon cancer (FIG. 5A-5B).

### Definitions

The articles “a” and “an” are used herein to refer to one or to more than one (i.e. to at least one) of the grammatical object of the article. By way of example, “an element” means one element or more than one element.

As used herein, an “antibody” or “antigen-binding polypeptide” refers to a polypeptide or a polypeptide complex that specifically recognizes and binds to an antigen. An antibody can be a whole antibody and any antigen binding fragment or a single chain thereof. Thus the term “antibody” includes any protein or peptide containing molecule that comprises at least a portion of an immunoglobulin molecule having biological activity of binding to the antigen. Examples of such include, but are not limited to a complementarity determining region (CDR) of a heavy or light chain or a ligand binding portion thereof, a heavy chain or light chain variable region, a heavy chain or light chain constant region, a framework (FR) region, or any portion thereof, or at least one portion of a binding protein.

The terms “antibody fragment” or “antigen-binding fragment”, as used herein, is a portion of an antibody such as F(ab′)₂, F(ab)₂, Fab′, Fab, Fv, scFv and the like. Regardless of structure, an antibody fragment binds with the same antigen that is recognized by the intact antibody. The term “antibody fragment” includes aptamers, spiegelmers, and diabodies. The term “antibody fragment” also includes any synthetic or genetically engineered protein that acts like an antibody by binding to a specific antigen to form a complex.

The term antibody encompasses various broad classes of polypeptides that can be distinguished biochemically. Those skilled in the art will appreciate that heavy chains are classified as gamma, mu, alpha, delta, or epsilon (γ, μ, α, δ, ε) with some subclasses among them (e.g., γ1-γ4). It is the nature of this chain that determines the “class” of the antibody as IgG, IgM, IgA IgG, or IgE, respectively. The immunoglobulin subclasses (isotypes) e.g., IgG1, IgG2, IgG3, IgG4, IgG5, etc. are well characterized and are known to confer functional specialization. Modified versions of each of these classes and isotypes are readily discernable to the skilled artisan in view of the instant disclosure and, accordingly, are within the scope of the instant disclosure. All immunoglobulin classes are clearly within the scope of the present disclosure, the following discussion will generally be directed to the IgG class of immunoglobulin molecules. With regard to IgG, a standard immunoglobulin molecule comprises two identical light chain polypeptides of molecular weight approximately 23,000 Daltons, and two identical heavy chain polypeptides of molecular weight 53,000-70,000. The four chains are typically joined by disulfide bonds in a “Y” configuration wherein the light chains bracket the heavy chains starting at the mouth of the “Y” and continuing through the variable region.

Antibodies, antigen-binding polypeptides, variants, or derivatives thereof of the disclosure include, but are not limited to, polyclonal, monoclonal, multispecific, human, humanized, primatized, or chimeric antibodies, single chain antibodies, epitope-binding fragments, e.g., Fab, Fab′ and F(ab′)₂, Fd, Fvs, single-chain Fvs (scFv), single-chain antibodies, disulfide-linked Fvs (sdFv), fragments comprising either a VK or VH domain, fragments produced by a Fab expression library, and anti-idiotypic (anti-Id) antibodies (including, e.g., anti-Id antibodies to LIGHT antibodies disclosed herein). Antibody portions, such as Fab and F(ab′)2 fragments, can be prepared from whole antibodies using conventional techniques, such as papain or pepsin digestion, respectively, of whole antibodies. Moreover, antibodies, antibody portions and immunoadhesion polypeptides can be obtained using standard recombinant DNA techniques. Immunoglobulin or antibody molecules of the disclosure can be of any type (e.g., IgG, IgE, IgM, IgD, IgA, and IgY), class (e.g., IgG1, IgG2, IgG3, IgG4, IgA1 and IgA2) or subclass of immunoglobulin molecule.

Light chains are classified as either kappa or lambda (K, λ). Each heavy chain class may be bound with either a kappa or lambda light chain. In general, the light and heavy chains are covalently bonded to each other, and the “tail” portions of the two heavy chains are bonded to each other by covalent disulfide linkages or non-covalent linkages when the immunoglobulins are generated either by hybridomas, B cells or genetically engineered host cells. In the heavy chain, the amino acid sequences run from an N-terminus at the forked ends of the Y configuration to the C-terminus at the bottom of each chain.

Both the light and heavy chains are divided into regions of structural and functional homology. The terms “constant” and “variable” are used functionally. In this regard, it will be appreciated that the variable domains of both the light (VK) and heavy (VH) chain portions determine antigen recognition and specificity. Conversely, the constant domains of the light chain (CK) and the heavy chain (CH1, CH2 or CH3) confer important biological properties such as secretion, transplacental mobility, Fc receptor binding, complement binding, and the like. By convention the numbering of the constant region domains increases as they become more distal from the antigen-binding site or amino-terminus of the antibody. The N-terminal portion is a variable region and at the C-terminal portion is a constant region; the CH3 and CK domains actually comprise the carboxy-terminus of the heavy and light chain, respectively.

As indicated above, the variable region allows the antibody to selectively recognize and specifically bind epitopes on antigens. That is, the VK domain and VH domain, or subset of the complementarity determining regions (CDRs), of an antibody combine to form the variable region that defines a three-dimensional antigen-binding site. This quaternary antibody structure forms the antigen-binding site present at the end of each arm of the Y. More specifically, the antigen-binding site is defined by three CDRs on each of the VH and VK chains (i.e. CDR-H1, CDR-H2, CDR-H3, CDR-L1, CDR-L2 and CDR-L3). In some instances, e.g., certain immunoglobulin molecules derived from camelid species or engineered based on camelid immunoglobulins, a complete immunoglobulin molecule may consist of heavy chains only, with no light chains.

Antibodies disclosed herein may be from any animal origin including birds and mammals. Preferably, the antibodies are human, murine, donkey, rabbit, goat, guinea pig, camel, llama, horse, or chicken antibodies. In other embodiments, the variable region may be condricthoid in origin (e.g., from sharks).

The amount of a biomarker (e.g., PLXDC1 or PLXDC2) in a subject is “significantly” higher or lower than the normal amount of the biomarker, if the amount of the biomarker is greater or less, respectively, than the normal or control level by an amount greater than the standard error of the assay employed to assess amount, and preferably at least 20%, 30%, 40%, 50%, 60%, 70%, 80%, 90%, 100%, 150%, 200%, 300%, 350%, 400%, 500%, 600%, 700%, 800%, 900%, 1000% or than that amount. Alternatively, the amount of the biomarker in the subject can be considered “significantly” higher or lower than the normal and/or control amount if the amount is at least about two, and preferably at least about 5%, 10%, 15%, 20%, 25%, 30%, 35%, 40%, 45%, 50%, 55%, 60%, 65%, 70%, 75%, 80%, 85%, 90%, 95%, 100%, 105%, 110%, 115%, 120%, 125%, 130%, 135%, 140%, 145%, 150%, 155%, 160%, 165%, 170%, 175%, 180%, 185%, 190%, 195%, two times, three times, four times, five times, or more, or any range in between, such as 5%-100%, higher or lower, respectively, than the normal and/or control amount of the biomarker. Such significant modulation values can be applied to any metric described herein, such as altered level of expression, altered activity, changes in cancer cell hyperproliferative growth, changes in cancer cell death, changes in biomarker inhibition, changes in test agent binding, and the like.

The “amount” of a marker, e.g., expression or copy number of a marker or MCR, or protein level of a marker, in a subject is “significantly” higher or lower than the normal amount of a marker, if the amount of the marker is greater or less, respectively, than the normal level by an amount greater than the standard error of the assay employed to assess amount, and preferably at least twice, and more preferably three, four, five, ten or more times that amount. Alternately, the amount of the marker in the subject can be considered “significantly” higher or lower than the normal amount if the amount is at least about two, and preferably at least about three, four, or five times, higher or lower, respectively, than the normal amount of the marker.

The term “altered level of expression” of a marker refers to an expression level or copy number of a marker in a test sample e.g., a sample derived from a subject suffering from cancer, that is greater or less than the standard error of the assay employed to assess expression or copy number, and is preferably at least twice, and more preferably three, four, five or ten or more times the expression level or copy number of the marker or chromosomal region in a control sample (e.g., sample from a healthy subject not having the associated disease) and preferably, the average expression level or copy number of the marker or chromosomal region in several control samples. The altered level of expression is greater or less than the standard error of the assay employed to assess expression or copy number, and is preferably at least twice, and more preferably three, four, five or ten or more times the expression level or copy number of the marker in a control sample (e.g., sample from a healthy subject not having the associated disease) and preferably, the average expression level or copy number of the marker in several control samples.

The terms “high,” “low,” “intermediate,” and “negative” in connection with cellular biomarker expression refers to the amount of the biomarker expressed relative to the cellular expression of the biomarker by one or more reference cells. Biomarker expression can be determined according to any method described herein including, without limitation, an analysis of the cellular level, activity, structure, and the like, of one or more biomarker genomic nucleic acids, ribonucleic acids, and/or polypeptides. In some embodiments, the terms refer to a defined percentage of a population of cells expressing the biomarker at the highest, intermediate, or lowest levels, respectively. Such percentages can be defined as the top 0.1%, 0.5%, 1.0%, 1.5%, 2.0%, 2.5%, 3.0%, 3.5%, 4.0%, 4.5%, 5.0%, 5.5%, 6.0%, 6.5%, 7.0%, 7.5%, 8.0%, 8.5%, 9.0%, 9.5%, 10%, 11%, 12%, 13%, 14%, 15% or more, or any range in between, inclusive, of a population of cells that either highly express or weakly express the biomarker. The term “low” excludes cells that do not detectably express the biomarker, since such cells are “negative” for biomarker expression. The term “intermediate” includes cells that express the biomarker, but at levels lower than the population expressing it at the “high” level. In other embodiments, the terms can also refer to, or in the alternative refer to, cell populations of biomarker expression identified by qualitative or statistical plot regions. For example, cell populations sorted using flow cytometry can be discriminated on the basis of biomarker expression level by identifying distinct plots based on detectable moiety analysis, such as based on mean fluorescence intensities and the like, according to well-known methods in the art. Such plot regions can be refined according to number, shape, overlap, and the like based on well-known methods in the art for the biomarker of interest. In still other embodiments, the terms can also be determined according to the presence or absence of expression for additional biomarkers.

By “specifically binds” or “has specificity to,” it is generally meant that an antibody binds to an epitope via its antigen-binding domain, and that the binding entails some complementarity between the antigen-binding domain and the epitope. According to this definition, an antibody is said to “specifically bind” to an epitope when it binds to that epitope, via its antigen-binding domain more readily than it would bind to a random, unrelated epitope. The term “specificity” is used herein to qualify the relative affinity by which a certain antibody binds to a certain epitope. For example, antibody “A” may be deemed to have a higher specificity for a given epitope than antibody “B,” or antibody “A” may be said to bind to epitope “C” with a higher specificity than it has for related epitope “D.”

As used herein, the terms “treat” or “treatment” refer to both therapeutic treatment and prophylactic or preventative measures, wherein the object is to prevent or slow down (lessen) an undesired physiological change or disorder, such as the progression of cancer. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

The term “preventing” is art-recognized, and when used in relation to a condition, such as a local recurrence, is well understood in the art, and includes administration of a composition which reduces the frequency of, or delays the onset of, symptoms of a medical condition in a subject relative to a subject which does not receive the composition. Thus, prevention of cancer includes, for example, reducing the number of detectable cancerous growths in a population of patients receiving a prophylactic treatment relative to an untreated control population, and/or delaying the appearance of detectable cancerous growths in a treated population versus an untreated control population, e.g., by a statistically and/or clinically significant amount.

The term “prophylactic” or “therapeutic” treatment is art-recognized and includes administration to the host of one or more of the subject compositions. If it is administered prior to clinical manifestation of the unwanted condition (e.g., disease or other unwanted state of the host animal) then the treatment is prophylactic (i.e., it protects the host against developing the unwanted condition), whereas if it is administered after manifestation of the unwanted condition, the treatment is therapeutic (i.e., it is intended to diminish, ameliorate, or stabilize the existing unwanted condition or side effects thereof).

By “subject” or “individual” or “animal” or “patient” or “mammal,” is meant any subject, particularly a mammalian subject, for whom diagnosis, prognosis, or therapy is desired. Mammalian subjects include humans, domestic animals, farm animals, and zoo, sport, or pet animals such as dogs, cats, guinea pigs, rabbits, rats, mice, horses, cattle, cows, and so on.

As used herein, phrases such as “to a patient in need of treatment” or “a subject in need of treatment” includes subjects, such as mammalian subjects, that would benefit from administration of an antibody or composition of the present disclosure used, e.g., for detection, for a diagnostic procedure and/or for treatment.

The terms “cancer” or “tumor” or “hyperproliferative disorder” refer to the presence of cells possessing characteristics typical of cancer-causing cells, such as uncontrolled proliferation, immortality, metastatic potential, rapid growth and proliferation rate, and certain characteristic morphological features. Cancer cells are often in the form of a tumor, but such cells may exist alone within an animal, or may be a non-tumorigenic cancer cell, such as a leukemia cell. Cancers include, but are not limited to, B cell cancer, e.g., multiple myeloma, Waldenström's macroglobulinemia, the heavy chain diseases, such as, for example, alpha chain disease, gamma chain disease, and mu chain disease, benign monoclonal gammopathy, and immunocytic amyloidosis, melanomas, breast cancer, lung cancer, bronchus cancer, colorectal cancer, prostate cancer, pancreatic cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, cancer of hematologic tissues, and the like. Other non-limiting examples of types of cancers applicable to the methods encompassed by the present invention include human sarcomas and carcinomas, e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, colorectal cancer, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, liver cancer, choriocarcinoma, seminoma, embryonal carcinoma, Wilms' tumor, cervical cancer, bone cancer, brain tumor, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma; leukemias, e.g., acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenstrom's macroglobulinemia, and heavy chain disease. In some embodiments, cancers are epithlelial in nature and include but are not limited to, bladder cancer, breast cancer, cervical cancer, colon cancer, gynecologic cancers, renal cancer, laryngeal cancer, lung cancer, oral cancer, head and neck cancer, ovarian cancer, pancreatic cancer, prostate cancer, or skin cancer. In other embodiments, the cancer is breast cancer, prostate cancer, lung cancer, or colon cancer. In still other embodiments, the epithelial cancer is non-small-cell lung cancer, nonpapillary renal cell carcinoma, cervical carcinoma, ovarian carcinoma (e.g., serous ovarian carcinoma), or breast carcinoma. The epithelial cancers may be characterized in various other ways including, but not limited to, serous, endometrioid, mucinous, clear cell, Brenner, or undifferentiated.

The “normal” level of expression of a marker is the level of expression of the marker in cells of a subject, e.g., a human patient, not afflicted with a disease or disorder related to aberrant marker levels. An “over-expression” or “significantly higher level of expression” of a marker refers to an expression level in a test sample that is greater than the standard error of the assay employed to assess expression, and is preferably at least twice, and more preferably three, four, five or ten times the expression level of the marker in a control sample (e.g., sample from a healthy subjects not having the marker associated disease) and preferably, the average expression level of the marker in several control samples. A “significantly lower level of expression” of a marker refers to an expression level in a test sample that is at least twice, and more preferably three, four, five or ten times lower than the expression level of the marker in a control sample (e.g., sample from a healthy subject not having the marker associated disease) and preferably, the average expression level of the marker in several control samples.

Such antibodies, described herein, can be used in any one of well-known immunoassay forms, including, without limitation, a radioimmunoassay, a Western blot assay, an immunofluorescence assay, an enzyme immunoassay, an immunoprecipitation assay, a chemiluminescence assay, an immunohistochemical assay, a dot blot assay, or a slot blot assay. General techniques to be used in performing the various immunoassays noted above and other variations of the techniques, such as in situ proximity ligation assay (PLA), fluorescence polarization immunoassay (FPIA), fluorescence immunoassay (FIA), enzyme immunoassay (EIA), nephelometric inhibition immunoassay (NIA), enzyme linked immunosorbent assay (ELISA), and radioimmunoassay (RIA), ELISA, etc. alone or in combination or alternatively with NMR, MALDI-TOF, LC-MS/MS, are known to those of ordinary skill in the art.

Such reagents can also be used to monitor protein levels in a cell or tissue, as part of a clinical testing procedure, e.g., in order to monitor an optimal dosage of an inhibitory agent. Detection can be facilitated by coupling (e.g., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, 35S or 3H.

Such reagents can also be used with any number of biological samples. Biological samples can be collected from a variety of sources from a patient including a body fluid sample, cell sample, or a tissue sample comprising nucleic acids and/or proteins. In preferred embodiments, the subject and/or control sample is selected from cells, cell lines, histological slides, paraffin embedded tissues, biopsies, whole blood, nipple aspirate, serum, plasma, buccal scrape, saliva, cerebrospinal fluid, urine, stool, and bone marrow. In certain embodiments, the sample is serum, plasma, or urine. In other embodiments, the sample is serum.

The samples can be collected from individuals repeatedly over a longitudinal period of time (e.g., once or more on the order of days, weeks, months, annually, biannually, etc.). Obtaining numerous samples from an individual over a period of time can be used to verify results from earlier detections and/or to identify an alteration in biological pattern as a result of, for example, disease progression, drug treatment, etc. For example, subject samples can be taken and monitored every month, every two months, or combinations of one, two, or three month intervals according to the present invention. In addition, the biomarker amount and/or activity measurements of the subject obtained over time can be conveniently compared with each other, as well as with those of normal controls during the monitoring period, thereby providing the subject's own values, as an internal, or personal, control for long-term monitoring.

Samples can contain live cells/tissue, fresh frozen cells, fresh tissue, biopsies, fixed cells/tissue, cells/tissue embedded in a medium, such as paraffin, histological slides, or any combination thereof.

Sample preparation and separation can involve any of the procedures, depending on the type of sample collected and/or analysis of biomarker measurement(s). Such procedures include, by way of example only, concentration, dilution, adjustment of pH, removal of high abundance polypeptides (e.g., albumin, gamma globulin, and transferrin, etc.), addition of preservatives and calibrants, addition of protease inhibitors, addition of denaturants, desalting of samples, concentration of sample proteins, extraction and purification of lipids.

The sample preparation can also isolate molecules that are bound in non-covalent complexes to other protein (e.g., carrier proteins). This process may isolate those molecules bound to a specific carrier protein (e.g., albumin), or use a more general process, such as the release of bound molecules from all carrier proteins via protein denaturation, for example using an acid, followed by removal of the carrier proteins.

### Detection of PLXDC1 and PLXDC2 Proteins in Tumor Samples

An important factor in successful identification of new antibodies is the presentation of an antigen to be accessible by the antibody. This is relatively easy for soluble proteins, but membrane proteins present significant challenges for antibody discovery. Even after an antibody is obtained, its use for detecting the expression of its target membrane protein can still be challenging. This is at least because, first, the epitope used for identifying the antibody may not be sufficiently exposed on the cell surface in the tissue to allow binding. Second, the conventional tissue slide preparation methods can mask the antibody epitopes by chemical crosslinking.

Through trial and error, and unexpected discoveries, the instant inventors have developed a tissue preparation method for immunohistochemistry that is distinct from known methods of immunohistochemistry for fresh human tissues. In certain embodiments, the present technology preserves the tissue morphology and does not mask antibody epitopes by chemical crosslinking using standard fixatives such as paraformaldehyde or formaldehyde. In various embodiments of the present technology, the tissue is fixed with a non-crosslinking fixative after cryostat sectioning. The non-crosslinking fixative is carried out for at least 30 minutes, 2 hours or 5 hours, preferably overnight.

Furthermore, specifically for the human Plexin domain containing 1 (PLXDC1, or TEM7) and Plexin domain containing 2 (PLXDC2) proteins, the instant inventors were able to identify, with structural modeling and laboratory testing, an epitope fragment from each protein that is effective in screening for antibodies capable of binding the target protein on fixed fresh tumor tissue samples. Moreover, even though PLXDC1 (NCBI Reference Sequence: NP_065138.2) and PLXDC2 (NCBI Reference Sequence: NP 116201.7) are highly homologous (see alignment in Table 1), these antibodies were able to specifically recognize the respective target protein without cross-reacting to the other.

TABLE 1

| Alignment of Human PLXDC1 and PLXDC2 Protein Sequences | | |
|---|---|---|
| Alignment (residues in epitopes are underlined; | | |
| PLXDC: SEQ ID NO:3, PLXDC: SEQ ID NO: 4) | | |
| PLXDC1 | ---------MRGELWLLVLVLREAARALSPQPGAGHDEGPGSGWA--------------- | 36 |
| PLXDC2 | MARFPKADLAAAGVMLLCHFFTDQFQFADGKPGD-----QILDWQYGVTQAFPHTEEEVE | 55 |
| | . : ** .: : : . :** .* | |
| PLXDC1 | --AKGTVRGWNRRARESPGHVSEPDRTQLSQDLG----GGTLAMDTLPDNRTR-VVEDNH | 89 |
| PLXDC2 | VDSHAYSHRWKRNLDELK--AVDTNRASVGQDSPEPRSFTDLLLDDGQDNNTQIEEDTDH | 113 |
| | ::. : *:*. . : :*:.:.:** * :* **.*: : :* | |
| PLXDC1 | SYYVSRLYGPSEPHSRELWVDVAEANRSQVKIHTILSNTHRQASRVVLSFDFPFYGHPLR | 149 |
| PLXDC2 | NYYISRIYGPSDSASRDLWVNIDQMEKDKVKIHGILSNTHRQAARVNLSFDFPFYGHFLR | 173 |
| | .**:**:****: **:***:: : ::.:**** *********:** ********** ** | |
| PLXDC1 | QITIATGGFIFMGDVIHRMLTATQYVAPLMANFNPGYSDNSTVVYFDNGTVFVVQWDHVY | 209 |
| PLXDC2 | EITVATGGFIYTGEVVHRMLTATQYIAPLMANFDPSVSRNSTVRYFDNGTALVVQWDHVH | 233 |
| | :**:******: *:*:*********:*******:*. * **** ******.********: | |
| PLXDC1 | LQGWEDKGSFTFQAALHHDGRIVFAYKEIPMSVPEISSSQHPVKTGLSDAFMILNPSPDV | 269 |
| PLXDC2 | LQDNYNLGSFTFQATLLMDGRIIFGYKEIPVLVTQISSTNHPVKVGLSDAFVVVHRIQQI | 293 |
| | **. : *******:* ****:*.*****: * :***::****.******:::: :: | |
| PLXDC1 | PESRRRSIFEYHRIELDPSKVTSMSAVEFTPLPTCLQHRSCDACMSSDLTFNCSWCHVLQ | 329 |
| PLXDC2 | PNVRRRTIYEYHRVELQMSKITNISAVEMTPLPTCLQFNRCGPCVSSQIGFNCSWCSKLQ | 353 |
| | *: ***:*:****:**: **:*.:****:*******.. *. *:**:: ****** ** | |
| PLXDC1 | RCSSGFDRYRQEWMDYGCAQEAEGRMCEDFQDEDHDSASPDT------SFSPYDGDLTTT | 383 |
| PLXDC2 | RCSSGFDRHRQDWVDSGCPEESKEKMCENTEPVETSSRTTTTVGATTTQFRVLT-TTRRA | 412 |
| | ********:**:*:* ** :*:: :***: : : .* : * .* : | |
| PLXDC1 | SSSLFIDSLITEDDTKLNPYAGGDGLQ-NNLSPKTKGTPVHLGTIVGIVLAVLLVAAIIL | 442 |
| PLXDC2 | VTSQFPTSLPTEDDTKIALHLKDNGASTDDSAAEKKGGTLHAGLIIGILILVLIVATAIL | 472 |
| | :* * ** ******: : .:* . :: : : :** :* * *:**:: **:**: ** | |
| PLXDC1 | AGIYINGHPTSNAALFFIERRPHHWPAMKFRSHPDHSTYAEVEPSGHEKEGFMEAEQC | 500 |
| PLXDC2 | VTVYMYHHPTSAASIFFIERRPSRWPAMKFRRGSGHPAYAEVEPVG-EKEGFIVSEQC | 529 |
| | . :*: **** *::******* :******* .* :****** * *****: :*** | |

PLXDC1 has been demonstrated to express in certain tumor types, and thus is a promising target for tumor detection and treatment. The role of PLXDC2 in tumorigenesis, however, is not well understood. As shown in the experimental examples, with the improved tumor tissue fixation methodology and using the newly developed antibodies, the instant inventors were able to detect the expression of PLXDC2 protein in tumor samples. Both proteins were highly enriched in tumor blood vessels in certain tumor samples.

### Anti-PLXDC1 and Anti-PLXDC2 Antibodies

PLXDC1 (TEM7) and PLXDC2 are both cell-surface receptors for Pigment Epithelium-Derived Factor (PEDF). PEDF was originally identified as a strong protective factor for neurons and was initially known as EPC-1, a factor that is downregulated by more than 100-fold in aged compared to young human fibroblasts. PEDF was identified as a potent endogenous inhibitor of angiogenesis. PEDF inhibits endothelial cell migration and angiogenesis even in the presence of strong proangiogenic factors. It specifically targets new vessel growth without affecting pre-existing vessels. In numerous animal models, PEDF has been shown to have potent therapeutic effects in treating several major human diseases through its neurotrophic, anti-angiogenic, antitumorigenic and antimetastatic activities.

The present invention relates, in part, to antibodies or fragments thereof that are directed against PLXDC1 and/or PLXDC2 (such as polyclonal antibodies listed herein). Such molecules, in part, are characterized in that they exhibit the ability to recognize PLXDC1 and/or PLXDC2 protein in diagnostic assays, such as immunohistochemical (IHC), Western blot, intercellular flow, ELISA, and the like. Such molecules, in part, are characterized in that they exhibit the ability to inhibit PLXDC1 and/or PLXDC2 activity.

The term “PLXDC1”, also known as plexin domain containing 1, Tumor endothelial marker 3, Tumor endothelial marker 7, TEM3 and TEM7, refers to a cell-surface receptor for Pigment Epithelium-Derived Factor (PEDF). PLXDC1 structures and functions, are well-known in the art as described above (see, for example, Beaty et al. (2007) J Neurooncol. 81(3):241-8, Lee et al. (2006) FEBS Lett. 580(9):2253-7, and Cheng et al. (2014) Elife. 3:e05401).

The term “PLXDC1” is intended to include fragments, variants (e.g., allelic variants), and derivatives thereof. Representative human PLXDC1 cDNA and human PLXDC1 protein sequences are well-known in the art and are publicly available from the National Center for Biotechnology Information (NCBI). Human PLXDC1 variants include variant 1 (NM_020405.5 and NP_065138.2). Nucleic acid and polypeptide sequences of PLXDC1 orthologs in organisms other than humans are well-known and include, for example, chimpanzee PLXDC1 (XM_016930792.2 and XP 016786281.1, XM_016930794.2 and XP_016786283.1, XM_016930791.2 and XP_016786280.1, XM_016930788.2 and XP_016786277.1, XM_016930790.2 and XP_016786279.1, and XM_016930789.2 and XP_016786278.1), dog PLXDC1 (XM_548152.3 and XP_548152.2, and XM_022423025.1 and XP_022278733.1), cattle PLXDC1 (NM_001099077.1 and NP_001092547.1), mouse PLXDC1 (NM_001163608.1 and NP_001157080.1, and NM_028199.3 and NP_082475.3), rat PLXDC1 (NM_001107046.1 and NP_001100516.1), chicken PLXDC1 (XM_015299524.2 and XP_015155010.1, XM_015299523.2 and XP_015155009.1), and frog PLXDC1 (XM_012952930.1 and XP_012808384.1, and XM_004918671.2 and XP_004918728.1).

The term “PLXDC2”, also known as plexin domain containing 2, Tumor Endothelial Marker 7-Related Protein, TEM3 and TEM7R, refers to a cell-surface receptor for Pigment Epithelium-Derived Factor (PEDF). PLXDC2 structures and functions, are well-known in the art as described above (see, for example, Miller et al. (2007) Gene Expr Patterns. 7(5):635-44, Miller-Delaney et al. (2011) PLoS One. 6(1):e14565, and Cheng et al. (2014) Elife. 3:e05401).

The term “PLXDC2” is intended to include fragments, variants (e.g., allelic variants), and derivatives thereof. Representative human PLXDC2 cDNA and human PLXDC2 protein sequences are well-known in the art and are publicly available from the National Center for Biotechnology Information (NCBI). Human PLXDC2 variants include variant 1 (NM_032812.9 and NP_116201.7, which represents the longer transcript and encodes the longer isoform 1), variant 2(NM_001282736.1 and NP_001269665.1, which lacks an alternate in-frame exon in the 5′ coding region, compared to variant 1). Nucleic acid and polypeptide sequences of PLXDC2 orthologs in organisms other than humans are well-known and include, for example, chimpanzee PLXDC2 (XM_024346245.1→XP_024202013.1), Rhesus monkey PLXDC2 (XM_001094803.4 and XP_001094803.1, and XM_028826043.1 and XP_028681876.1), dog PLXDC2 (XM_022406830.1 and XP_022262538.1, XM_022406817.1 and XP_022262525.1, XM_845716.5 and XP_850809.2, XM_022406828.1 and XP_022262536.1, and XM_022406819.1 and XP_022262527.1), cattle PLXDC2 (XM_025000576.1 and XP_024856344.1, XM_025000574.1 and XP_024856342.1, XM_005214189.4 and XP_005214246.1, and XM_015473986.2 and XP_015329472.1), mouse PLXDC2 (NM_026162.6 and NP_080438.2), Norway rat PLXDC2 (NM_001108422.2 and NP_001101892.1), chicken PLXDC2 (XM_418613.6 and XP_418613.2), frog PLXDC2 (XM_002933138.3 and XP_002933184.1), and zebrafish PLXDC2 (NM_001146305.1 and NP_001139777.1).

Nucleic acid and amino acid sequence information for nucleic acid and polypeptide molecules useful in the present invention are well-known in the art and readily available on publicly available databases, such as the National Center for Biotechnology Information (NCBI). For example, exemplary nucleic acid and amino acid sequences derived from publicly available sequence databases are provided in Table 1 below.

```
TABLE 1
SEQ ID NO: 5
Human PLXDC1 cDNA Sequence (NM_020405.5, CDS region
from position 201-1703)
        1 gctcccggag gccgcagcct ccagctccgc tcgcgctctc gccgctcctg ccggctcgcc
      61 cggccccgcg ctccgccgtc tcctcgccgc ccgcccctcc gccagccccg gggaccgcgc
    121 ggccgcagcc tgagccaggg ccccctccct cgtcaggacc ggggcagcaa gcaggccggg
    181 ggcaggtccg ggcacccacc atgcgaggcg agctctggct cctggtgctg gtgctcaggg
    241 aggctgcccg ggcgctgagc ccccagcccg gagcaggtca cgatgagggc ccaggctctg
    301 gatgggctgc caaagggacc gtgcggggct ggaaccggag agcccgagag agccctgggc
    361 atgtgtcaga gccggacagg acccagctga gccaggacct gggtgggggc accctggcca
    421 tggacacgct gccagataac aggaccaggg tggtggagga caaccacagc tattatgtgt
    481 cccgtctcta tggccccagc gagccccaca gccgggaact gtgggtagat gtggccgagg
    541 ccaaccggag ccaagtgaag atccacacaa tactctccaa cacccaccgg caggcttcga
    601 gagtggtctt gtcctttgat ttccctttct acgggcatcc tctgcggcag atcaccatag
    661 caactggagg cttcatcttc atgggggacg tgatccatcg gatgctcaca gctactcagt
    721 atgtggcgcc cctgatggcc aacttcaacc ctggctactc cgacaactcc acagttgttt
    781 actttgacaa tgggacagtc tttgtggttc agtgggacca cgtttatctc caaggctggg
    841 aagacaaggg cagtttcacc ttccaggcag ctctgcacca tgacggccgc attgtctttg
    901 cctataaaga gatccctatg tctgtcccgg aaatcagctc ctcccagcat cctgtcaaaa
    961 ccggcctatc ggatgccttc atgattctca atccatcccc ggatgtgcca gaatctcggc
  1021 gaaggagcat ctttgaatat caccgcatag agctggaccc cagcaaggtc accagcatgt
  1081 cggccgtgga gttcacccca ttgccgacct gcctgcagca taggagctgt gacgcctgca
  1141 tgtcctcaga cctgaccttc aactgcagct ggtgccatgt cctccagaga tgctccagtg
  1201 gctttgaccg ctatcgccag gagtggatgg actatggctg tgcacaggag gcagagggca
  1261 ggatgtgcga ggacttccag gatgaggacc acgactcagc ctcccctgac acttccttca
  1321 gcccctatga tggagacctc accactacct cctcctccct cttcatcgac agcctcacca
  1381 cagaagatga caccaagttg aatccctatg caggaggaga cggccttcag aacaacctgt
  1441 cccccaagac aaagggcact cctgtgcacc tgggcaccat cgtgggcatc gtgctggcag
  1501 tcctcctcgt ggcggccatc atcctggctg gaatttacat caatggccac cccacatcca
  1561 atgctgcgct cttcttcatc gagcgtagac ctcaccactg gccagccatg aagtttcgca
  1621 gccaccctga ccattccacc tatgcggagg tggagccctc gggccatgag aaggagggct
  1681 tcatggaggc tgagcagtgc tgagaacacc aagtctcccc tttgaagact ttgaggccac
  1741 agaaaagaca gttaaagcaa agaagagaag tgacttttcc tggcctctcc cagcatgccc
  1801 tgggctgaga tgagatggtg gtttatggct ccagagctgc tgctcgcttc gtcagcacac
  1861 cccgaatatt gaagaggggg ccaaaaaaca accacatgga ttttttatag gaacaacaac
  1921 ctaatctcat cctgttttga tgcaagggtt ctcttctgtg tcttgtaacc atgaaacagc
  1981 agaagaacta acataactaa ctccattttt gtttaagggg cctttaccta ttcctgcacc
  2041 taggctagga taactttaga gcactgacat aaaacgcaaa aacaggaatc atgccgtttg
  2101 caaaactaac tctgggatta aaggggaagc atgtaaacag ctaactgttt ttgttaaaga
  2161 tttataggaa tgaggaggtt tggctattgt cacatgacag actgttagcc aaggacaaag
  2221 aagttctgca aacctcccct ggacccttgc tggtgtccag atgtctgcgg ttgtcagccc
  2281 cttcctttcc cccgacctaa acataaaaga caaggcaaag cccgcataat tttaagacgg
  2341 ttctttagga cattagtcca ccatcttctt ggtttgctgg ctctccgaaa taaagtccct
  2401 ttccttgctc caactccttg tctctcaacg tattggctat gacgcagcaa gcagaatgaa
  2461 tttggactca gttacaggct gtcaatggtc tgctctgtag cagtctcaga gcctccccga
  2521 cccactacct ggagatagcc agatagccag atgccctgct cctggccacc tttaaagccc
  2581 ctgcatatga cacaggttaa ctaaagtcaa gattggggct gctgcattcc aggttcccta
  2641 gactcacaag ctggtccttg gccaggtgca gtggctcacg cctgtaatcc cagcactttg
  2701 ggaggctgag gcaggcggat cacctgaagt cagaagtttg agaccagcct ggccaacata
  2761 attaaaatgt ctctactaaa aatacaaaaa attagctggg tgtggtgacg cttgcctgta
  2821 tcccagctac tcaggaagct gagacacgag aatcacttga acctgggagg cagaggttgc
  2881 agtgagctca gatagtgcca ctgcactcca gcctgggtga cagagcgaga ctccgtctca
  2941 aaaaaaaaaa aagaaagcag atcctcatgg ctatagagtt ggcattttag ccccagcttc
  3001 tgtagctctg aaagcctaaa gaaggtattc tctccatctg ttaaacacag tatagtggct
  3061 ctcagccctt ggggcatgtt atcatgggag ggaagtcaaa taagaggaga gaaaagaact
  3121 caagggggaa actgcatttt taggctttgc tctcttacct tgccctttct actcagaacc
  3181 aataacttct gcatcaaaac atgttacagc ctgcatcaag ggctttaccc caacctgcag
  3241 cccagccttc cctgggtgag cttgctatgc gcagccacat ttaccatgtg gggctcccta
  3301 ttctgatggc ctgttcggtg ccgggtttac tcactgccct gttctgatgt cagtgcctgt
  3361 acatacctcc aaaggcagga cttgcctgat aaatattttt cctcctctga actggatttt
  3421 ataggcatta aagacaagtc gggtggctag agggctcctt gagacatacc tagcagggaa
  3481 ctgcaggtgg attctgttga gaggcaaagc acctgagtgg ttgggacaca ggcagctggc
  3541 atgggaggga ctttttttga gacagggtct cactgtgtcg cccagggcaa ggatgcccaa
  3601 agacaccagg ttggagaggc acctgccaac tacttgcttt ccctggagcc tgcatgtgcc
  3661 tgtggggtgg ggaggcgtag gggtctacgg ctgcctgaga tgggtgtgca cagtgtgtga
  3721 agtacctacc tccttgcctt gctggactgt cagccagtcg cagggccggc cacaagaccc
  3781 atgtctccat ctggtcatac tccatagcta ccaagttaac ctgctctaaa ctttggagaa
  3841 ctggatctgt ccaataaacg cttatttggc caagcctgat ggctcgtgcc tgtactccca
  3901 gcactttggg aggctgaggt gggagggttg cttgagccca ggggtttgag accagcttgg
  3961 gcaacaacaa caaaaatgcc aggtgtggtg gggtgcacct gtagtcccag ctactaggga
  4021 ggctgagcca ggaggatcac ttgagcccgg gaggttgagg ctgcagtggg gggtcataat
  4081 catgccactg tactccagcc tgggtgacag agtgagaccc tgtctccgaa aaaaaaaaaa
  4141 aaaaaaagaa cggaaaaaga aatgcttaca ttgtcaggga tcctgtagac aatcattaac
  4201 tctatgagat gcttggttct atttttttgg gagactttgt ccaagtgttt tggcttaaga
  4261 aatccatagg cctctcttgg tgacacatct ctagtacttt ttgtcataaa caaacaggcc
  4321 atctgccgcc aaatacatcc actccccatg ccactgacat cctatgggtc agccaggctt
  4381 gctttgactg aggccgaggc atctggaact ttctctgcct gcaggggcta gcagcagagg
  4441 cttcaccgca tcaccacccc ttcctccact cctgacattc tttcccttca gggatccaaa
  4501 atggttggcc gagctcccag tgggaaaacg tgtgctagag ttggggagtg agatgagtgg
  4561 tgctgtccat ggaatcaggc cacagcagga actgccccac tggccatttg agacacacac
  4621 aggtggtaaa tgctctgctg gtgggctgtg cttccctcat tcagagagct ctgttacagc
  4681 ccactgtgtc ctttagaagc ttgaaaggaa cccaactctt tgctgcactg tcctttttct
  4741 tcctcaaatt cagaccctcc ttccaccggc acccccctac tccaccctca gctcttcctt
  4801 gcctggttta tcaagcagag ctgaggcccc acgtttccaa ctctgattgt cacttgcatc
  4861 ttcacaaagg ataaaccacg gagcaactgg aaaaccatca gccaagcgtt cggatgagtc
  4921 tggttattgg tccacccccg accagattcc cttacactta actcacttct ttctttggca
  4981 atgaccctca tgacatgtat aaatgggtat gactaagaag aggctgtgat ctaacattta
  5041 tttgctgcca ttttttactc tggggagaag cagccccaac tcatcactgg gaaagaactc
  5101 cccctgcaaa ccagctaaat ttgataattt aaaccccctg cccctaaaac ttctcacaga
  5161 gctggggagt tggtggcaac tttccaagtc aaggtcttgc ttagaaagtc cttcactaca
  5221 tggccaggtg cagtggctca cgcctgtagt cccaggtact tgggagcctg aggcaggagg
  5281 attgcttgag ctcaggagtt caaggctgca gagagctatg atcatcccac tgcatttgtt
  5341 taaaaataaa tttttaaaat ttgtgtgttt tatcaggggt ctcctgtaca gtgtatctgt
  5401 gtatgtttgt gtgtgtgttt gtatacagcc ttgtttaatg ttttgagcaa taagatatgc
  5461 acacacaggt attttgttgc taaagagatt ggacaaggtt gtagctgtgc tcaggcttca
  5521 gcttggtttg ttaaattgag agataaacaa tgacaagagc tgccagccaa ccacactatt
  5581 caaaaagcaa agtgttcacc actaaagcta accattcatc tggttgcagg caaggctaag
  5641 gctctctctc ctctagttcc tggaacagac tcacagattg gcatgaagca ctgatcaggg
  5701 gctgcactca gactccctgg ccaagcaaac ctacaccaga agagtcagtg tcacagatat
  5761 gatgcggcca atctctgtct ccaaaaacct acctgaactt aatggtagaa ttcaaagatc
  5821 tggggactga gggcacccag ccttctaaaa cacaatgtat tcatgtgttt agtgtaaact
  5881 ctctgcatgg attctcagtg ttaataataa aaggaagcat tcttttacaa ctcctgctgt
  5941 gtgcaaaaga aagtgcaaag gatttggagt ggcattccga agatcaccac acataccttg
  6001 gttctgatgg ctgctgaact ccgacttctt cgctgagaca tgactgtggg aacagcctcc
  6061 agctatctgc tcatcagagg tgctttcctc aacctcctgc accacctcca agagaaacag
  6121 cctaaaaaga aaccccagct gtttacttat attggtctgt aaatccctgg aagtaaaccc
  6181 catgcatttt tatctactgt ctgaggacat acaataaatc tgagaaagtc
SEQ ID NO: 6
Human PLXDC1 Amino Acid Sequence (NP_065138.2)
        1 mrgelwllvl vlreaarals pqpgaghdeg pgsgwaakgt vrgwnrrare spghvsepdr
      61 tqlsqdlggg tlamdtlpdn rtrvvednhs yyvsrlygps ephsrelwvd vaeanrsqvk
    121 ihtilsnthr qasrvvlsfd fpfyghplrq itiatggfif mgdvihrmlt atqyvaplma
    181 nfnpgysdns tvvyfdngtv fvvqwdhvyl qgwedkgsft fqaalhhdgr ivfaykeipm
    241 svpeisssqh pvktglsdaf milnpspdvp esrrrsifey hrieldpskv tsmsaveftp
    301 lptclqhrsc dacmssdltf ncswchvlqr cssgfdryrq ewmdygcage aegrmcedfq
    361 dedhdsaspd tsfspydgdl tttssslfid sltteddtkl npyaggdglq nnlspktkgt
    421 pvhlgtivgi vlavllvaai ilagiyingh ptsnaalffi errphhwpam kfrshpdhst
    481 yaevepsghe kegfmeaeqc
SEQ ID NO: 7
Human PLXDC2 cDNA Sequence variant 1 (NM_032812.9, CDS
region from position 649-2238)
        1 agactgctgc agccctaacc ttcccagggc tcagctcttt ggagctgccc attcctccgg
      61 ctgcgagaaa ggacgcgcgc cctgcgtcgg gcgaagaaaa gaagcaaaac ttgtcgggag
    121 ggtttcgtca tcaacctcct tcccgcaaac ctaaacctcc tgccggggcc atccctagac
    181 agaggaaagt tcctgcagag ccgaccagcc ctagtggatc tggggcaggc agcggcgctg
    241 gctgtggaat tagatctgtt ttgaacccag tggagcgcat cgctggggct cggaagtcac
    301 cgtccgcggg caccgggttg gcgctgcccg agtggaaccg acagtttgcg agcctcggct
    361 gcaagtggcc tctcctcccc gcggttgttg ttcagtgtcg ggtgagggct gcgagtgtgg
    421 caagttgcaa agagagcctc agaggtccga agagcgctgc gctcctactc gcgttcgctt
    481 cttcctcttc tcggttccct actgtgaaat cgcagcgaca tttacaaagg cctccgggtc
    541 ctaccgagac cgatccgcag cgtttggccc ggtcgtgcct attgcatcgg gagcccccga
    601 gcaccggcga aggactggcg ggtggggtag ggaggtggcg gcggcggcat ggcgaggttc
    661 ccgaaggccg acctggccgc tgcaggagtt atgttacttt gccacttctt cacggaccag
    721 tttcagttcg ccgatgggaa acccggagac caaatccttg attggcagta tggagttact
    781 caggccttcc ctcacacaga ggaggaggtg gaagttgatt cacacgcgta cagccacagg
    841 tggaaaagaa acttggactt tctcaaggcg gtagacacga accgagcaag cgtcggccaa
    901 gactctcctg agcccagaag cttcacagac ctgctgctgg atgatgggca ggacaataac
    961 actcagatcg aggaggatac agaccacaat tactatatat ctcgaatata tggtccatct
  1021 gattctgcca gccgggattt atgggtgaac atagaccaaa tggaaaaaga taaagtgaag
  1081 attcatggaa tattgtccaa tactcatcgg caagctgcaa gagtgaatct gtccttcgat
  1141 tttccatttt atggccactt cctacgtgaa atcactgtgg caaccggggg tttcatatac
  1201 actggagaag tcgtacatcg aatgctaaca gccacacagt acatagcacc tttaatggca
  1261 aatttcgatc ccagtgtatc cagaaattca actgtcagat attttgataa tggcacagca
  1321 cttgtggtcc agtgggacca tgtacatctc caggataatt ataacctggg aagcttcaca
  1381 ttccaggcaa ccctgctcat ggatggacga atcatctttg gatacaaaga aattcctgtc
  1441 ttggtcacac agataagttc aaccaatcat ccagtgaaag tcggactgtc cgatgcattt
  1501 gtcgttgtcc acaggatcca acaaattccc aatgttcgaa gaagaacaat ttatgaatac
  1561 caccgagtag agctacaaat gtcaaaaatt accaacattt cggctgtgga gatgacccca
  1621 ttacccacat gcctccagtt taacagatgt ggcccctgtg tatcttctca gattggcttc
  1681 aactgcagtt ggtgtagtaa acttcaaaga tgttccagtg gatttgatcg tcatcggcag
  1741 gactgggtgg acagtggatg ccctgaagag tcaaaagaga agatgtgtga gaatacagaa
  1801 ccagtggaaa cttcttctcg aaccaccaca accgtaggag cgacaaccac ccagttcagg
  1861 gtcctaacta ccaccagaag agcagtgact tctcagtttc ccaccagcct ccctacagaa
  1921 gatgatacca agatagcact acatctaaaa gataatggag cttctacaga tgacagtgca
  1981 gctgagaaga aagggggaac cctccacgct ggcctcatca ttggaatcct catcctggtc
  2041 ctcattgtag ccacagccat tcttgtgaca gtctatatgt atcaccaccc aacatcagca
  2101 gccagcatct tctttattga gagacgccca agcagatggc ctgcgatgaa gtttagaaga
  2161 ggctctggac atcctgccta tgctgaagtt gaaccagttg gagagaaaga aggctttatt
  2221 gtatcagagc agtgctaaaa tttctaggac agaacaacac cagtactggt ttacaggtgt
  2281 taagactaaa attttgccta tacctttaag acaaacaaac aaacacacac acaaacaagc
  2341 tctaagctgc tgtagcctga agaagacaag atttctggac aagctcagcc caggaaacaa
  2401 agggtaaaca aaaaactaaa acttatacaa gataccattt acactgaaca tagaattccc
  2461 tagtggaatg tcatctatag ttcactcgga acatctcccg tggacttatc tgaagtatga
  2521 caagattata atgcttttgg cttaggtgca gggttgcaaa gggatcagaa aaaaaaaatc
  2581 ataataaagc tttagttcat gagggatcga cacctttggt tcaaatgttc tctgatgtct
  2641 caaagataac tgttttccaa agcctgaacc ctttcactca aaagagcaat gatgaatgtc
  2701 tcaagattgc taagaaaaac agcccatgca agagtgagaa caaacacaaa ataagagatt
  2761 ttctacattt tcaaaacaga tgtgtggcaa aaggatgttg tttttctggt ctagatccat
  2821 ctgtaccaac aagttcatca ctttacagaa cgaatctttt tatccgtaca ggaggttcaa
  2881 accatgtctg cctcttcctt tgtaatgaat gacctttcta tgagctgtga caaaatttcc
  2941 gaacaattaa ctaaggattt gggaagaggg ggtggcaaac ggggctttct gttttcctgc
  3001 ctcagcatga aaacatctga tttatgcttt atggaagcct tacctccaat ccccaactgt
  3061 taagtcccat gaaaccacag ttgctctggg ctgatggaaa caaaaggaaa cagtatgaag
  3121 agttccttaa tcatttttga aacaaaaatg ttaagggatt ttaaacatat gattattttt
  3181 aattttatgc cttttcagta ctaaacaccc atttcattgc tgattcctgt ctaagaagcc
  3241 attcacgtca gcatggcgat agaaagaatg aaaaaaccct gctgaatcat acagtaattt
  3301 tctttaaagc acatagtagt tacataaata tatatatata aatatatttt tgtttataac
  3361 taacacaagg caggatcttg tgactctaag agtgcgtttt gtcatcaagg caaaacagat
  3421 gcaagatgca tcactgcatt acttccatag agttgtaaaa taatccttaa tattagaata
  3481 tttttctgtc acttagcaaa agtggttcag ttcattgccg cgcccatcat gttcttgact
  3541 atttgatcca ctttttcgtt tatgtcaacc ccttccctct ctggctaaat aaagtggatg
  3601 cagaaagctc cttaaatgga gatatcgatt gccttggaat cacaatcctg attttgaaaa
  3661 ttcctcatga atgaagaaag gaatggcatc ccttgagaag gaaagtggtt aatatacata
  3721 ctgagctcct aaagtttaaa ttcaggtact gagtgtacaa tttcaccaac attctaaccc
  3781 atgaaacttt tacactctgt gccaagaaac tgttggcttt tgtaaggtac agtgctcaac
  3841 atttgcagat tcaggtctca agaagcagag atgtctcata agcagcattt tcccaacagt
  3901 ttagcatctg tacacatctg ccttggtcat cagtccactc acagagtacc atactttatc
  3961 atcacaagtg tctgacgtga acgaatgcca ttttctattc catatatttt gctttacaat
  4021 tttaagtatt tgatgaagat ggtaactttt tcctaactta gttaactatt aaaaaaaatt
  4081 ttgaaaagca aggtgattga aggattgtga tgacaatctc tttgcagcag ctatgtatgg
  4141 tttatgtgaa gtatccccac ttattcttgt ggagcaggtt tggtgagaca gcaataacca
  4201 aatgacatgc caatattact ggtgcaactg gtattctaca aatgcataag gaacacatag
  4261 acgacttcct tttaggataa aatgatgctt ctttcactac cttttgtggt agctgtggct
  4321 tccaatagca actgtttgac agttatataa atcttgcatg tgtattctta gtttgtgtcc
  4381 cttaagtact acttaattct caagtagtaa tgttattctt atacccttca gcgttctatt
  4441 ttgattcaaa acaattgatt caaaacaatt ttgaatcaat tttctatttt gattcaaaac
  4501 aattgataat tctgtaaaaa aacataaaca ctgaattctt cagtgaacca aagcaacaaa
  4561 taatagagaa aacttcttga aactggagtg tgggaaaact tcttaacaga actaagagtt
  4621 aaaggtagtg agaagtgtgt ggtgtgtgaa ttctttagtg gtaaggggaa atgtgggcta
  4681 aatccttttc tttcatgaaa ctctcattct attttatatt tctggtttgt tcttgttccc
  4741 atgtgagaaa acatacagtt tctgaaaatt caaaatggtc atcaatgctt tggactttac
  4801 ataattatgt attagagaag gtgcaactgt acattactta atatactatg aacataatag
  4861 aataacaaaa aaagataaca aagagatgca aacttctgga taaatcagat aaatggtgct
  4921 acagaggaat ttagttattt cagcttaatt atttgtaaaa ataataatca gtgactaggt
  4981 aaagatactg aaactcaaga aaaatatctt gacccattta tgtctagtgt tccattattg
  5041 gaacgctaag cttatgggag ttatttagag cctactgctc aaggtcatca ccaaggtctg
  5101 gttgcaaaaa ttcaaaaaat tgcaacctca ggcataaatg gcttaagccc aagagatggc
  5161 actgaccata aatggtggct tgtgtgtgaa gggtttggac ttcaagatat actaacacct
  5221 agaaaactaa aagtgcaatt gacccccaat tactatttta gtttgaggta tcaagggcat
  5281 ttgccaatct cttatatgta gcttctagag ttgtgctgtc caatattata tccactagcc
  5341 aacgtggctt ttgaaattaa aattaattaa aattaaatta aaaatgtaat tcttcagtca
  5401 caccagccac atttcaaagg ctcaatagcc acgcctggct tgtggctacc atattggata
  5461 acaagaaaca cttccgtggt tgaagaaaat tttaatggac aatgctgtgt ttcatttgaa
  5521 ttcgttgtcc ttgaagaaaa gggaagaaga ggaaagaaac aagatttggt agctcttggg
  5581 catatccaag catttttgat tttagagagg ccctatggag taatgaaaaa aatacaagct
  5641 gacacagctt taaatcccca ctgtgttatc ttgggaaaat cacttcaacc tccttgagtc
  5701 tcaatttgca attttggaaa agaaaaacaa tttttgtcct gtctttcttg caagattatt
  5761 atgcaacctg attaaatgta cagatgtgca gactcttaaa aaccttatgt taatgtaacc
  5821 catataaagt acgacttttg gtcgagtacc agcacaaatt tgcattcaaa ggaagaatag
  5881 ttcatcagtg caaaaagcgt tcaaaggtaa tcagttcatc acgtatcttg agaaaaagag
  5941 aatgcattca aaacacaaag caaaattgct tcagtttctt ttctgcccat ctgaatattt
  6001 cctttagtat ttgtcccaca tgacatccat cccatgtatt tatccactct ttcacaacat
  6061 tgcctatgaa catccttcaa ctggctcagt tctttattgc atagagccac ttagcttctc
  6121 tagtttctta tgatttccta ttttacactt aacagcaaaa ggaggcttcc atttagaagg
  6181 cactatctca aacataaata atttcatatt tattttatca ctgtaacact aaggagctat
  6241 ggcattaaaa tataaaacta tttggaactt aatgtaaacc tttgcacatg cagactatat
  6301 aaagggaata aaattatttt aaattatgct aatatccaga tacatattct aaggttagta
  6361 tcatttatca gtttttcgca acagattctt atgttacatc ttcatgaggg agtataaatt
  6421 tgatcacgtc cctagtgtct attgcccact tgagaaaggt tttgccagga tccacagatc
  6481 gcttcaagat gctttcgttt atgataggaa attaatattt aaataagact aattgaaaac
  6541 tagattatct ggttctaatt tcttcctcct gtgaaatgtt cttttggttt gtatttcttt
  6601 aggtattttc caagttactg ctgcttttac ttcacgacct ccaaccccac ttttgttatc
  6661 agcgtcatat gccagagtca tggcagatcc agcctggaca acatagtgag acccatctct
  6721 atcaaatata catatatata tatatatata cacacacaca cacacacaca caaatataca
  6781 tatatatcaa atatatatgt atatttgata gagatgatag caattatgtg tgtgtgtgtg
  6841 tgtgtgtgtg tgtgtgtgtt agccaagtgt ggtttctcat gcctgtagtc ccagctactt
  6901 tggaggctga agtgtaagag ttgcttgagc ccatgagtta gaggctgctg tgagctatga
  6961 tcatgccact gcactccagc ctgggcaaca gagcaaaacc ccatctctaa ataaaaaaaa
  7021 agaatcgtga gacatcatgt aatccaacct cttaatgtta cagataagta aactgaggcc
  7081 caaagagata tagtaatagt cccagtgtca gtaaactggg tcttgaactt aaatgtttgt
  7141 ttcagttctc attgccttca tctcttaatt gtcggcactt aaggccaacc aagcagctta
  7201 gacagcgcag tccactcaac ggtaacttta tttgctatat tgacaatctg ctctgttctg
  7261 taatatagaa cattggatca atctttatct ttttgctgac ttaaggaggc tgctattgtt
  7321 gcaatatcca gagatttcaa gttctagcca ttgcggggtc tttgtggtgt tttcaatttc
  7381 tcctctctcc acccctgccc ccatctgtgg ccatctaagt tagcaataca atttattttg
  7441 gccatttaaa atttttctgc ctttgtttta tttcttccca aatatatttg aaaatgaatg
  7501 atctatatag ctgattttct gaccacatat ataattgcaa tttttatttg ctcttcaaac
  7561 tattacaaca tttttgtcct tataaggttt tttgggcaga taatgcgagg tctgtggtgt
  7621 taccttttat atactcctat taccttatgt ttgtgaagca ttttacattt tacagagcac
  7681 tttgattcat ctccctattt gttcatttat ttaactgttc agttcactga aatatttatt
  7741 gaacacctac tggaaccaag tgactgtgct agtaacttgt gatatacagt agtgagagtc
  7801 caggcatgta accgatcatt ataatacaat aacatcccta aaccagactt tacaagtaga
  7861 taggatcaac aatactctat caattttcaa ctccaatttt ctattctact taaaatggga
  7921 actagaaact gtttgcctga agaatgtgtc tgaaacataa tatatcactg catgtctgtg
  7981 ctgtagacct gttaatttta tctgtgagaa aaaaagttac tcaaaattct ccctgaccta
  8041 agaatacttt ctagttcact gcagtcttac tgaccagatg caacagttga agtttgattt
  8101 ctcgacccaa tatttctatt gggttttgaa ttattaatat cactgttttg aggtattcag
  8161 aaacaccagt gtatcaaaaa agcatttgca ctttaggtgt gtgtggtggt tatgtcattt
  8221 attagaccat cccagacata agacaatcag ggaaatcaga aaactccagc ctcaaatgtg
  8281 tctataattt cctgttctac cattgtcata tcataacatg gtattacttc ttaaggtttt
  8341 gattaagttg atctagcctc aacttaaatt gtaatacatc tgcctaatta ttgtctggaa
  8401 taacttttca accatccaat gcccactgct ctcacaatga ttattgtaga gaagtaaaat
  8461 ggtaattatt caagtaaagt cacataattt ctggagtcag tttttcataa caagtttatg
  8521 gaatacatca tcattggctt cttcataata tatttattat gagtgaccaa attttgcctg
  8581 gaggagcaaa atgctcaaaa cttgttatta taggttaatt tccagctcac tgttgacact
  8641 gaaagattct gtgttacttt aaacccagga taaaaggctg gaaaaaaaat taaatgtaag
  8701 tcataaacta gtactcagct tttcctagtt tctaaggctt attaacattt gcaaattact
  8761 caataaatgt ctttcataat ggaataacat aaaagctttt gatttggcag atagtgatat
  8821 tttatttatt ttcattctgg ttgaaaaaaa tctcagtggc ttctcttcat tccacgagaa
  8881 ttttgatttt taacagcagt ctctcttttt ctcagcattg caaatatata tgtatatata
  8941 cattcatgac caaagtatcg cttactgacc atgcagctgt aaaccttctg tgcctatcaa
  9001 acaaatacat agcatgaaac taattttaga agtttcatgg gggaatttta ggggaaagta
  9061 taaacctaag agtgagtgaa tggagatgat tcatggaaaa aaaaataaaa atctaaatgt
  9121 gctattaggc agagttatta acttctttta gttgttgttt gagatggggt tttgctcttg
  9181 ttacccaggc tggagtgcaa tggcgtgatc tcgtctcact gcaacctccg cctcccaggt
  9241 tcaagcgatt ctcctgcctc agccgcccaa gtagctggga ttacaggcat atgccactac
  9301 agccggctaa ttttgtattt ttagtagaga cggggtttct ccatgctggt caggctggtc
  9361 cccagctccc gacctcaggt gatccgccca cctccgcctc ccaaagtgct ggaattacag
  9421 gcctgagcca ccgtgcctgg ccgagttatt aacttcttaa gagcaatgtg ctaataaata
  9481 ctcattgatg accagctcaa atttaggtca ttcagacatc cagacactgg ggcacatatt
  9541 ctgcaagcaa tgctgagacc cctgacatag agaaagcaaa ggatatgcct atgattagtc
  9601 taaaatgcag ccatcacccc ccatacctct tctatggcat tcatcctaac atcatggagg
  9661 cctttgtcct agagaattat gtgacttgcc ctagagaatt agtgaatgac caaaaagaga
  9721 ctttccattt atcttccttt gacttaaaag gcatgaaaat aaggcaaaaa aatcaataaa
  9781 ataattttcc tgaggaaagg ttaagagatg gcactttcct ttccgggccc agagctggat
  9841 ttctctgaga tttgaccatc tcaggactca gacagactcc agtgctattt tctggacatt
  9901 tgtggtgaag cctcagccac catgttcaag gtggtttgaa tgaaaacata ctcagattgt
  9961 cacatttata gcagggaaat aaaaactcaa attaggcatc ctgcagcagg ctactctgaa
10021 aataacatta ggcaacatca gagcttcact tgcaaagaaa tgttaaaatc attttaggga
10081 aatcagtgaa gtctctttag aaacagacat cttgtgtatg gcgtaaccca gtcttggggc
10141 cctcacggag aagaggggga agtcttttca ttgattggtc aaaacaaatc tctcatttgc
10201 ctgatttgaa gcatttcaac agtgcctgga gagtacatta ttttcagggg aaaaaaagga
10261 gagagtcttt tttatatgcc agctgggatc atgggaactt cgaatgccag gaatttacta
10321 ctgtttctgg taattctgtg tgtagtcatt tgaaatgttg aagtgtgaaa agagaagaaa
10381 ttgggcactt cttgcggcgg gggagggggg gggggcggtg gctttccaga ttttatgcca
10441 gttgcaccag catgcagaat atttgtaatg catttcaaag tggatataat ggcacccttt
10501 gtcagaatca caaagctcac tgcggcactg ctacaagagg acactgagga aaatctggcc
10561 ctatgaacct agtcaacccc aagcaaaaag aatgactatg tgtgtgagtg cagcacatgg
10621 ccagttcgtt tctcactgtt ttggaaagcc ctgtgtgcca aaccaaggac gtgtctttca
10681 gggaaaggtt aattttccga agtttattaa aatagaactt ggaaaaccaa gcattttgaa
10741 tttattccag tcctctgggc atcattccta tttcttctgc catgtcaagg agaaattcca
10801 agcctgcatt ctgtcatgct aaaataacca gcccatactt ctcggtgacc ttctgttgaa
10861 cgtacctgag cctgcaaatg taaaaatgat tgtatctgaa tctgcactaa tggtgtctga
10921 gagcaaaaag agtgtgacct ctattggaaa cctttgttca aattcaataa ttcagagatg
10981 ctacatactt ctgcaagctt cctgattatg ttcactgtaa tattaatgac ctaagtttga
11041 atgtatttcc ttacagtcca ttaatttgac atccatcttt tacctgggga ttattacaat
11101 tgcaataagt cattaatgtt ttcttcacac agcttcttaa accaagtttc tctgcagctc
11161 tttcggttct gcttacagtg tgtgggaaat ctgatttttt tcccctagta atagtttgat
11221 aagaaattta gtgtattgac tgcctcagtg acacaattta tctttaaagg tgtggaagct
11281 ggtggggacc aaatgttacc tgtgtttttg ctgttgattg ctattttcag aagcaaacca
11341 tgtttttcac ttacagtagg agtcaacaaa tttgggattt tagaaggggg aggagggagc
11401 tatttgtgta agactgctgt catatttgac tacatattaa aaacagtaaa tgagcatttt
11461 gttttaattt cttaaatacc ttgtctttca acatacgttt tgtttccttt cttccattag
11521 tgttcaaaag gttctaccca ttgtggaaga aattctgtgt gcagaattca gaggcacaag
11581 gctgatggca agatagaaag ttattttgct tctaaaccca ccccgatgtg gaaactgata
11641 ctagctagag ggagctgtag aaaacaaaga tttcaggatt gcacagtgtg tgggcaatgg
11701 gatggagact ttttccccta ttcccagcca cagtgcccaa gcgttcaagt ctcctggatc
11761 agacagatgg gattttagct gctgctttaa atcctagtgc tggaataagt caaggtactt
11821 cagttcagct cttgcctctg tcactaatct tgctttatga actcctttga ttttctgaat
11881 aagttccaga aggttctcta ttattctgtc cttcttccaa actggaaatg gctgtatcta
11941 attctcagga tattttggat gtgtgcctca ggtaatttat gtggaatgtg taaagcaaga
12001 tgtctccaat tctgaatatt ccttcccctt ttcccaatcc tccactcttg gactaccttt
12061 ataacaacac cgagtacgca cagacctgaa cccatgccca agaagcacac acaatgactg
12121 gagctgtcgg gaattcctgt cagtggcatt ccctgagcac tggctctgta caactcaatt
12181 ataatttttt aagaatcata cctctgtata gatcttttgg actgtactga ttaaactttg
12241 atattgtgga gtaaattcag aagtgcaatt ttaaa
SEQ ID NO: 8 Human PLXDC2 Amino Acid Sequence variant 1 (NP_116201.7)
        1 marfpkadla aagvmllchf ftdqfqfadg kpgdqildwq ygvtqafpht eeevevdsha
      61 yshrwkrnld flkavdtnra svgqdspepr sftdlllddg qdnntqieed tdhnyyisri
    121 ygpsdsasrd lwvnidqmek dkvkihgils nthrqaarvn lsfdfpfygh flreitvatg
    181 gfiytgevvh rmltatqyia plmanfdpsv srnstvryfd ngtalvvqwd hvhlqdnynl
    241 gsftfqatll mdgriifgyk eipvlvtqis stnhpvkvgl sdafvvvhri qqipnvrrrt
    301 iyeyhrvelq mskitnisav emtplptclq fnrcgpcvss qigfncswcs klqrcssgfd
    361 rhrqdwvdsg cpeeskekmc entepvetss rttttvgatt tqfrvltttr ravtsqfpts
    421 lpteddtkia lhlkdngast ddsaaekkgg tlhagliigi lilvlivata ilvtvymyhh
    481 ptsaasiffi errpsrwpam kfrrgsghpa yaevepvgek egfivseqc
SEQ ID NO: 9 Human PLXDC2 cDNA Sequence variant 2 (NM_001282736.1,
CDS region from position 638-2080)
        1 gccctaacct tcccagggct cagctctttg gagctgccca ttcctccggc tgcgagaaag
      61 gacgcgcgcc ctgcgtcggg cgaagaaaag aagcaaaact tgtcgggagg gtttcgtcat
    121 caacctcctt cccgcaaacc taaacctcct gccggggcca tccctagaca gaggaaagtt
    181 cctgcagagc cgaccagccc tagtggatct ggggcaggca gcggcgctgg ctgtggaatt
    241 agatctgttt tgaacccagt ggagcgcatc gctggggctc ggaagtcacc gtccgcgggc
    301 accgggttgg cgctgcccga gtggaaccga cagtttgcga gcctcggctg caagtggcct
    361 ctcctccccg cggttgttgt tcagtgtcgg gtgagggctg cgagtgtggc aagttgcaaa
    421 gagagcctca gaggtccgaa gagcgctgcg ctcctactcg cgttcgcttc ttcctcttct
    481 cggttcccta ctgtgaaatc gcagcgacat ttacaaaggc ctccgggtcc taccgagacc
    541 gatccgcagc gtttggcccg gtcgtgccta ttgcatcggg agcccccgag caccggcgaa
    601 ggactggcgg gtggggtagg gaggtggcgg cggcggcatg gcgaggttcc cgaaggccga
    661 cctggccgct gcaggagtta tgttactttg ccacttcttc acggaccagt ttcagttcgc
    721 cgatgggaaa cccggagacc aaatccttga ttggcagtat ggagttactc aggccttccc
    781 tcacacagag gaggaggtgg aagttgattc acacgcgtac agccacaggt ggaaaagaaa
    841 cttggacttt ctcaaggcgg tagacacgaa ccgagcaagc gtcggccaag actctcctga
    901 gcccagaagc ttcacagacc tgctgctgga tgatgggcag gacaataaca ctcagatcga
    961 gagagtgaat ctgtccttcg attttccatt ttatggccac ttcctacgtg aaatcactgt
  1021 ggcaaccggg ggtttcatat acactggaga agtcgtacat cgaatgctaa cagccacaca
  1081 gtacatagca cctttaatgg caaatttcga tcccagtgta tccagaaatt caactgtcag
  1141 atattttgat aatggcacag cacttgtggt ccagtgggac catgtacatc tccaggataa
  1201 ttataacctg ggaagcttca cattccaggc aaccctgctc atggatggac gaatcatctt
  1261 tggatacaaa gaaattcctg tcttggtcac acagataagt tcaaccaatc atccagtgaa
  1321 agtcggactg tccgatgcat ttgtcgttgt ccacaggatc caacaaattc ccaatgttcg
  1381 aagaagaaca atttatgaat accaccgagt agagctacaa atgtcaaaaa ttaccaacat
  1441 ttcggctgtg gagatgaccc cattacccac atgcctccag tttaacagat gtggcccctg
  1501 tgtatcttct cagattggct tcaactgcag ttggtgtagt aaacttcaaa gatgttccag
  1561 tggatttgat cgtcatcggc aggactgggt ggacagtgga tgccctgaag agtcaaaaga
  1621 gaagatgtgt gagaatacag aaccagtgga aacttcttct cgaaccacca caaccgtagg
  1681 agcgacaacc acccagttca gggtcctaac taccaccaga agagcagtga cttctcagtt
  1741 tcccaccagc ctccctacag aagatgatac caagatagca ctacatctaa aagataatgg
  1801 agcttctaca gatgacagtg cagctgagaa gaaaggggga accctccacg ctggcctcat
  1861 cattggaatc ctcatcctgg tcctcattgt agccacagcc attcttgtga cagtctatat
  1921 gtatcaccac ccaacatcag cagccagcat cttctttatt gagagacgcc caagcagatg
  1981 gcctgcgatg aagtttagaa gaggctctgg acatcctgcc tatgctgaag ttgaaccagt
  2041 tggagagaaa gaaggcttta ttgtatcaga gcagtgctaa aatttctagg acagaacaac
  2101 accagtactg gtttacaggt gttaagacta aaattttgcc tataccttta agacaaacaa
  2161 acaaacacac acacaaacaa gctctaagct gctgtagcct gaagaagaca agatttctgg
  2221 acaagctcag cccaggaaac aaagggtaaa caaaaaacta aaacttatac aagataccat
  2281 ttacactgaa catagaattc cctagtggaa tgtcatctat agttcactcg gaacatctcc
  2341 cgtggactta tctgaagtat gacaagatta taatgctttt ggcttaggtg cagggttgca
  2401 aagggatcag aaaaaaaaaa tcataataaa gctttagttc atgagggatc gacacctttg
  2461 gttcaaatgt tctctgatgt ctcaaagata actgttttcc aaagcctgaa ccctttcact
  2521 caaaagagca atgatgaatg tctcaagatt gctaagaaaa acagcccatg caagagtgag
  2581 aacaaacaca aaataagaga ttttctacat tttcaaaaca gatgtgtggc aaaaggatgt
  2641 tgtttttctg gtctagatcc atctgtacca acaagttcat cactttacag aacgaatctt
  2701 tttatccgta caggaggttc aaaccatgtc tgcctcttcc tttgtaatga atgacctttc
  2761 tatgagctgt gacaaaattt ccgaacaatt aactaaggat ttgggaagag ggggtggcaa
  2821 acggggcttt ctgttttcct gcctcagcat gaaaacatct gatttatgct ttatggaagc
  2881 cttacctcca atccccaact gttaagtccc atgaaaccac agttgctctg ggctgatgga
  2941 aacaaaagga aacagtatga agagttcctt aatcattttt gaaacaaaaa tgttaaggga
  3001 ttttaaacat atgattattt ttaattttat gccttttcag tactaaacac ccatttcatt
  3061 gctgattcct gtctaagaag ccattcacgt cagcatggcg atagaaagaa tgaaaaaacc
  3121 ctgctgaatc atacagtaat tttctttaaa gcacatagta gttacataaa tatatatata
  3181 taaatatatt tttgtttata actaacacaa ggcaggatct tgtgactcta agagtgcgtt
  3241 ttgtcatcaa ggcaaaacag atgcaagatg catcactgca ttacttccat agagttgtaa
  3301 aataatcctt aatattagaa tatttttctg tcacttagca aaagtggttc agttcattgc
  3361 cgcgcccatc atgttcttga ctatttgatc cactttttcg tttatgtcaa ccccttccct
  3421 ctctggctaa ataaagtgga tgcagaaagc tccttaaatg gagatatcga ttgccttgga
  3481 atcacaatcc tgattttgaa aattcctcat gaatgaagaa aggaatggca tcccttgaga
  3541 aggaaagtgg ttaatataca tactgagctc ctaaagttta aattcaggta ctgagtgtac
  3601 aatttcacca acattctaac ccatgaaact tttacactct gtgccaagaa actgttggct
  3661 tttgtaaggt acagtgctca acatttgcag attcaggtct caagaagcag agatgtctca
  3721 taagcagcat tttcccaaca gtttagcatc tgtacacatc tgccttggtc atcagtccac
  3781 tcacagagta ccatacttta tcatcacaag tgtctgacgt gaacgaatgc cattttctat
  3841 tccatatatt ttgctttaca attttaagta tttgatgaag atggtaactt tttcctaact
  3901 tagttaacta ttaaaaaaaa ttttgaaaag caaggtgatt gaaggattgt gatgacaatc
  3961 tctttgcagc agctatgtat ggtttatgtg aagtatcccc acttattctt gtggagcagg
  4021 tttggtgaga cagcaataac caaatgacat gccaatatta ctggtgcaac tggtattcta
  4081 caaatgcata aggaacacat agacgacttc cttttaggat aaaatgatgc ttctttcact
  4141 accttttgtg gtagctgtgg cttccaatag caactgtttg acagttatat aaatcttgca
  4201 tgtgtattct tagtttgtgt cccttaagta ctacttaatt ctcaagtagt aatgttattc
  4261 ttataccctt cagcgttcta ttttgattca aaacaattga ttcaaaacaa ttttgaatca
  4321 attttctatt ttgattcaaa acaattgata attctgtaaa aaaacataaa cactgaattc
  4381 ttcagtgaac caaagcaaca aataatagag aaaacttctt gaaactggag tgtgggaaaa
  4441 cttcttaaca gaactaagag ttaaaggtag tgagaagtgt gtggtgtgtg aattctttag
  4501 tggtaagggg aaatgtgggc taaatccttt tctttcatga aactctcatt ctattttata
  4561 tttctggttt gttcttgttc ccatgtgaga aaacatacag tttctgaaaa ttcaaaatgg
  4621 tcatcaatgc tttggacttt acataattat gtattagaga aggtgcaact gtacattact
  4681 taatatacta tgaacataat agaataacaa aaaaagataa caaagagatg caaacttctg
  4741 gataaatcag ataaatggtg ctacagagga atttagttat ttcagcttaa ttatttgtaa
  4801 aaataataat cagtgactag gtaaagatac tgaaactcaa gaaaaatatc ttgacccatt
  4861 tatgtctagt gttccattat tggaacgcta agcttatggg agttatttag agcctactgc
  4921 tcaaggtcat caccaaggtc tggttgcaaa aattcaaaaa attgcaacct caggcataaa
  4981 tggcttaagc ccaagagatg gcactgacca taaatggtgg cttgtgtgtg aagggtttgg
  5041 acttcaagat atactaacac ctagaaaact aaaagtgcaa ttgaccccca attactattt
  5101 tagtttgagg tatcaagggc atttgccaat ctcttatatg tagcttctag agttgtgctg
  5161 tccaatatta tatccactag ccaacgtggc ttttgaaatt aaaattaatt aaaattaaat
  5221 taaaaatgta attcttcagt cacaccagcc acatttcaaa ggctcaatag ccacgcctgg
  5281 cttgtggcta ccatattgga taacaagaaa cacttccgtg gttgaagaaa attttaatgg
  5341 acaatgctgt gtttcatttg aattcgttgt ccttgaagaa aagggaagaa gaggaaagaa
  5401 acaagatttg gtagctcttg ggcatatcca agcatttttg attttagaga ggccctatgg
  5461 agtaatgaaa aaaatacaag ctgacacagc tttaaatccc cactgtgtta tcttgggaaa
  5521 atcacttcaa cctccttgag tctcaatttg caattttgga aaagaaaaac aatttttgtc
  5581 ctgtctttct tgcaagatta ttatgcaacc tgattaaatg tacagatgtg cagactctta
  5641 aaaaccttat gttaatgtaa cccatataaa gtacgacttt tggtcgagta ccagcacaaa
  5701 tttgcattca aaggaagaat agttcatcag tgcaaaaagc gttcaaaggt aatcagttca
  5761 tcacgtatct tgagaaaaag agaatgcatt caaaacacaa agcaaaattg cttcagtttc
  5821 ttttctgccc atctgaatat ttcctttagt atttgtccca catgacatcc atcccatgta
  5881 tttatccact ctttcacaac attgcctatg aacatccttc aactggctca gttctttatt
  5941 gcatagagcc acttagcttc tctagtttct tatgatttcc tattttacac ttaacagcaa
  6001 aaggaggctt ccatttagaa ggcactatct caaacataaa taatttcata tttattttat
  6061 cactgtaaca ctaaggagct atggcattaa aatataaaac tatttggaac ttaatgtaaa
  6121 cctttgcaca tgcagactat ataaagggaa taaaattatt ttaaattatg ctaatatcca
  6181 gatacatatt ctaaggttag tatcatttat cagtttttcg caacagattc ttatgttaca
  6241 tcttcatgag ggagtataaa tttgatcacg tccctagtgt ctattgccca cttgagaaag
  6301 gttttgccag gatccacaga tcgcttcaag atgctttcgt ttatgatagg aaattaatat
  6361 ttaaataaga ctaattgaaa actagattat ctggttctaa tttcttcctc ctgtgaaatg
  6421 ttcttttggt ttgtatttct ttaggtattt tccaagttac tgctgctttt acttcacgac
  6481 ctccaacccc acttttgtta tcagcgtcat atgccagagt catggcagat ccagcctgga
  6541 caacatagtg agacccatct ctatcaaata tacatatata tatatatata tacacacaca
  6601 cacacacaca cacaaatata catatatatc aaatatatat gtatatttga tagagatgat
  6661 agcaattatg tgtgtgtgtg tgtgtgtgtg tgtgtgtgtg ttagccaagt gtggtttctc
  6721 atgcctgtag tcccagctac tttggaggct gaagtgtaag agttgcttga gcccatgagt
  6781 tagaggctgc tgtgagctat gatcatgcca ctgcactcca gcctgggcaa cagagcaaaa
  6841 ccccatctct aaataaaaaa aaagaatcgt gagacatcat gtaatccaac ctcttaatgt
  6901 tacagataag taaactgagg cccaaagaga tatagtaata gtcccagtgt cagtaaactg
  6961 ggtcttgaac ttaaatgttt gtttcagttc tcattgcctt catctcttaa ttgtcggcac
  7021 ttaaggccaa ccaagcagct tagacagcgc agtccactca acggtaactt tatttgctat
  7081 attgacaatc tgctctgttc tgtaatatag aacattggat caatctttat ctttttgctg
  7141 acttaaggag gctgctattg ttgcaatatc cagagatttc aagttctagc cattgcgggg
  7201 tctttgtggt gttttcaatt tctcctctct ccacccctgc ccccatctgt ggccatctaa
  7261 gttagcaata caatttattt tggccattta aaatttttct gcctttgttt tatttcttcc
  7321 caaatatatt tgaaaatgaa tgatctatat agctgatttt ctgaccacat atataattgc
  7381 aatttttatt tgctcttcaa actattacaa catttttgtc cttataaggt tttttgggca
  7441 gataatgcga ggtctgtggt gttacctttt atatactcct attaccttat gtttgtgaag
  7501 cattttacat tttacagagc actttgattc atctccctat ttgttcattt atttaactgt
  7561 tcagttcact gaaatattta ttgaacacct actggaacca agtgactgtg ctagtaactt
  7621 gtgatataca gtagtgagag tccaggcatg taaccgatca ttataataca ataacatccc
  7681 taaaccagac tttacaagta gataggatca acaatactct atcaattttc aactccaatt
  7741 ttctattcta cttaaaatgg gaactagaaa ctgtttgcct gaagaatgtg tctgaaacat
  7801 aatatatcac tgcatgtctg tgctgtagac ctgttaattt tatctgtgag aaaaaaagtt
  7861 actcaaaatt ctccctgacc taagaatact ttctagttca ctgcagtctt actgaccaga
  7921 tgcaacagtt gaagtttgat ttctcgaccc aatatttcta ttgggttttg aattattaat
  7981 atcactgttt tgaggtattc agaaacacca gtgtatcaaa aaagcatttg cactttaggt
  8041 gtgtgtggtg gttatgtcat ttattagacc atcccagaca taagacaatc agggaaatca
  8101 gaaaactcca gcctcaaatg tgtctataat ttcctgttct accattgtca tatcataaca
  8161 tggtattact tcttaaggtt ttgattaagt tgatctagcc tcaacttaaa ttgtaataca
  8221 tctgcctaat tattgtctgg aataactttt caaccatcca atgcccactg ctctcacaat
  8281 gattattgta gagaagtaaa atggtaatta ttcaagtaaa gtcacataat ttctggagtc
  8341 agtttttcat aacaagttta tggaatacat catcattggc ttcttcataa tatatttatt
  8401 atgagtgacc aaattttgcc tggaggagca aaatgctcaa aacttgttat tataggttaa
  8461 tttccagctc actgttgaca ctgaaagatt ctgtgttact ttaaacccag gataaaaggc
  8521 tggaaaaaaa attaaatgta agtcataaac tagtactcag cttttcctag tttctaaggc
  8581 ttattaacat ttgcaaatta ctcaataaat gtctttcata atggaataac ataaaagctt
  8641 ttgatttggc agatagtgat attttattta ttttcattct ggttgaaaaa aatctcagtg
  8701 gcttctcttc attccacgag aattttgatt tttaacagca gtctctcttt ttctcagcat
  8761 tgcaaatata tatgtatata tacattcatg accaaagtat cgcttactga ccatgcagct
  8821 gtaaaccttc tgtgcctatc aaacaaatac atagcatgaa actaatttta gaagtttcat
  8881 gggggaattt taggggaaag tataaaccta agagtgagtg aatggagatg attcatggaa
  8941 aaaaaaataa aaatctaaat gtgctattag gcagagttat taacttcttt tagttgttgt
  9001 ttgagatggg gttttgctct tgttacccag gctggagtgc aatggcgtga tctcgtctca
  9061 ctgcaacctc cgcctcccag gttcaagcga ttctcctgcc tcagccgccc aagtagctgg
  9121 gattacaggc atatgccact acagccggct aattttgtat ttttagtaga gacggggttt
  9181 ctccatgctg gtcaggctgg tccccagctc ccgacctcag gtgatccgcc cacctccgcc
  9241 tcccaaagtg ctggaattac aggcctgagc caccgtgcct ggccgagtta ttaacttctt
  9301 aagagcaatg tgctaataaa tactcattga tgaccagctc aaatttaggt cattcagaca
  9361 tccagacact ggggcacata ttctgcaagc aatgctgaga cccctgacat agagaaagca
  9421 aaggatatgc ctatgattag tctaaaatgc agccatcacc ccccatacct cttctatggc
  9481 attcatccta acatcatgga ggcctttgtc ctagagaatt atgtgacttg ccctagagaa
  9541 ttagtgaatg accaaaaaga gactttccat ttatcttcct ttgacttaaa aggcatgaaa
  9601 ataaggcaaa aaaatcaata aaataatttt cctgaggaaa ggttaagaga tggcactttc
  9661 ctttccgggc ccagagctgg atttctctga gatttgacca tctcaggact cagacagact
  9721 ccagtgctat tttctggaca tttgtggtga agcctcagcc accatgttca aggtggtttg
  9781 aatgaaaaca tactcagatt gtcacattta tagcagggaa ataaaaactc aaattaggca
  9841 tcctgcagca ggctactctg aaaataacat taggcaacat cagagcttca cttgcaaaga
  9901 aatgttaaaa tcattttagg gaaatcagtg aagtctcttt agaaacagac atcttgtgta
  9961 tggcgtaacc cagtcttggg gccctcacgg agaagagggg gaagtctttt cattgattgg
10021 tcaaaacaaa tctctcattt gcctgatttg aagcatttca acagtgcctg gagagtacat
10081 tattttcagg ggaaaaaaag gagagagtct tttttatatg ccagctggga tcatgggaac
10141 ttcgaatgcc aggaatttac tactgtttct ggtaattctg tgtgtagtca tttgaaatgt
10201 tgaagtgtga aaagagaaga aattgggcac ttcttgcggc gggggagggg gggggggcgg
10261 tggctttcca gattttatgc cagttgcacc agcatgcaga atatttgtaa tgcatttcaa
10321 agtggatata atggcaccct ttgtcagaat cacaaagctc actgcggcac tgctacaaga
10381 ggacactgag gaaaatctgg ccctatgaac ctagtcaacc ccaagcaaaa agaatgacta
10441 tgtgtgtgag tgcagcacat ggccagttcg tttctcactg ttttggaaag ccctgtgtgc
10501 caaaccaagg acgtgtcttt cagggaaagg ttaattttcc gaagtttatt aaaatagaac
10561 ttggaaaacc aagcattttg aatttattcc agtcctctgg gcatcattcc tatttcttct
10621 gccatgtcaa ggagaaattc caagcctgca ttctgtcatg ctaaaataac cagcccatac
10681 ttctcggtga ccttctgttg aacgtacctg agcctgcaaa tgtaaaaatg attgtatctg
10741 aatctgcact aatggtgtct gagagcaaaa agagtgtgac ctctattgga aacctttgtt
10801 caaattcaat aattcagaga tgctacatac ttctgcaagc ttcctgatta tgttcactgt
10861 aatattaatg acctaagttt gaatgtattt ccttacagtc cattaatttg acatccatct
10921 tttacctggg gattattaca attgcaataa gtcattaatg ttttcttcac acagcttctt
10981 aaaccaagtt tctctgcagc tctttcggtt ctgcttacag tgtgtgggaa atctgatttt
11041 tttcccctag taatagtttg ataagaaatt tagtgtattg actgcctcag tgacacaatt
11101 tatctttaaa ggtgtggaag ctggtgggga ccaaatgtta cctgtgtttt tgctgttgat
11161 tgctattttc agaagcaaac catgtttttc acttacagta ggagtcaaca aatttgggat
11221 tttagaaggg ggaggaggga gctatttgtg taagactgct gtcatatttg actacatatt
11281 aaaaacagta aatgagcatt ttgttttaat ttcttaaata ccttgtcttt caacatacgt
11341 tttgtttcct ttcttccatt agtgttcaaa aggttctacc cattgtggaa gaaattctgt
11401 gtgcagaatt cagaggcaca aggctgatgg caagatagaa agttattttg cttctaaacc
11461 caccccgatg tggaaactga tactagctag agggagctgt agaaaacaaa gatttcagga
11521 ttgcacagtg tgtgggcaat gggatggaga ctttttcccc tattcccagc cacagtgccc
11581 aagcgttcaa gtctcctgga tcagacagat gggattttag ctgctgcttt aaatcctagt
11641 gctggaataa gtcaaggtac ttcagttcag ctcttgcctc tgtcactaat cttgctttat
11701 gaactccttt gattttctga ataagttcca gaaggttctc tattattctg tccttcttcc
11761 aaactggaaa tggctgtatc taattctcag gatattttgg atgtgtgcct caggtaattt
11821 atgtggaatg tgtaaagcaa gatgtctcca attctgaata ttccttcccc ttttcccaat
11881 cctccactct tggactacct ttataacaac accgagtacg cacagacctg aacccatgcc
11941 caagaagcac acacaatgac tggagctgtc gggaattcct gtcagtggca ttccctgagc
12001 actggctctg tacaactcaa ttataatttt ttaagaatca tacctctgta tagatctttt
12061 ggactgtact gattaaactt tgatattgtg gagtaaattc agaagtgcaa ttttaaaaaa
12121 aaaaaaaaaa aaaaa
SEQ ID NO: 10
Human PLXDC2 Amino Acid Sequence variant 2
(NP_001269665.1)
        1 marfpkadla aagvmllchf ftdqfqfadg kpgdqildwq ygvtqafpht eeevevdsha
      61 yshrwkrnld flkavdtnra svgqdspepr sftdlllddg qdnntqierv nlsfdfpfyg
    121 hflreitvat ggfiytgevv hrmltatqyi aplmanfdps vsrnstvryf dngtalvvqw
    181 dhvhlqdnyn lgsftfqatl lmdgriifgy keipvlvtqi sstnhpvkvg lsdafvvvhr
    241 iqqipnvrrr tiyeyhrvel qmskitnisa vemtplptcl qfnrcgpcvs sqigfncswc
    301 sklqrcssgf drhrqdwvds gcpeeskekm centepvets srttttvgat ttqfrvlttt
    361 rravtsqfpt slpteddtki alhlkdngas tddsaaekkg gtlhagliig ililvlivat
    421 ailvtvymyh hptsaasiff ierrpsrwpa mkfrrgsghp ayaevepvge kegfivseqc
```

To prepare this polyclonal antibody, rabbits were immunized with recombinant gel-filtered PLXDC1, PLXDC2, or disclosed epitopes herein (SEQ ID NOs: 1 or 2) protein. Sera were incubated with GST-Sepharose 4B beads to remove contaminants, yielding the polyclonal antibodies in serum, as described by the applicants in Jun Yang et al., Molecular Cell (2002).

PLXDC1 and PLXDC2 both have extracellular, transmembrane and intracellular domains. With many years' experience in designing and generating antibodies for transmembrane proteins, the instant inventors were able to use structural modeling and laboratory testing to select, for each protein, a peptide sequence for antibody generation. For human PLXDC1, the peptide is SPQPGAGHDEGPGSGWAAKGTVRG (SEQ ID NO:1). For human PLXDC2, the peptide is KPGDQILDWQYGVTQAFPHTE (SEQ ID NO:2).

Each of these peptides was first conjugated to the keyhole limpet hemocyanin (KLH) and the peptide-KLH conjugate was used to immunize rabbits. After the fourth bleed, the rabbit sera were further affinity-purified using the peptide conjugated to Affigel (Biorad®). The purified antibodies were able to specifically recognize the corresponding target protein but did not bind to the other protein, despite their sequence homology (FIG. 1A-1D).

In accordance with one aspect of the present disclosure, therefore, provided is an antibody or antigen-binding fragment thereof having binding specificity to a human Plexin domain containing 1 (PLXDC1) protein, wherein the antibody or fragment thereof is capable of binding to at least one of the amino acid residues within SEQ ID NO:1 (SPQPGAGHDEGPGSGWAAKGTVRG). In some embodiments, the antibody or fragment thereof is capable of binding to at least two non-adjacent amino acid residues within SEQ ID NO:1. In some embodiments, the antibody or fragment thereof is capable of binding to at least three, four, five, six, seven, eight, nine, ten, twelve, fifteen or twenty amino acid residues within SEQ ID NO:1. In some embodiments, the binding between the antibody or fragment thereof and amino acid residues in SEQ ID NO:1 is sufficient to maintain the binding specificity between the antibody or fragment thereof and the PLXDC1 protein.

The antibody may be a polyclonal antibody or a monoclonal antibody. In some embodiments, the antibody or fragment thereof is obtained by immunizing an animal with a fusion protein comprising a fragment of PLXDC1 shorter than 50 amino acid residues and comprising at least 50% of SEQ ID NO:1. In some embodiments, the antibody or fragment thereof is obtained by phage display screened with a fragment of PLXDC1 shorter than 50 amino acid residues and comprising at least 50% of SEQ ID NO:1.

In some embodiments, the fragment includes at least 60%, 70%, 80% or 90% of SEQ ID NO: 1. In some embodiments, the fragment includes the entire SEQ ID NO: 1. In some embodiments, the fragment consists of SEQ ID NO:1.

In some embodiments, the fragment is conjugated to a carrier protein, such as KLH (keyhole lympet hemocyanin), BSA (bovine serum albumin), OVA (ovalbumin), and THY (thyroglobulin).

In accordance with another aspect of the present disclosure, therefore, provided is an antibody or antigen-binding fragment thereof having binding specificity to a human Plexin domain containing 2 (PLXDC2) protein, wherein the antibody or fragment thereof is capable of binding to at least one of the amino acid residues within SEQ ID NO:2 (KPGDQILDWQYGVTQAFPHTE). In some embodiments, the antibody or fragment thereof is capable of binding to at least two non-adjacent amino acid residues within SEQ ID NO:2. In some embodiments, the antibody or fragment thereof is capable of binding to at least three, four, five, six, seven, eight, nine, ten, twelve, fifteen or twenty amino acid residues within SEQ ID NO:2. In some embodiments, the binding between the antibody or fragment thereof and amino acid residues in SEQ ID NO:2 is sufficient to maintain the binding specificity between the antibody or fragment thereof and the PLXDC2 protein.

The antibody may be is a polyclonal antibody or a monoclonal antibody. In some embodiments, the antibody or fragment thereof is obtained by immunizing an animal with a fusion protein comprising a fragment of PLXDC2 shorter than 50 amino acid residues and comprising at least 50% of SEQ ID NO:2. In some embodiments, the antibody or fragment thereof is obtained by phage display screened with a fragment of PLXDC2 shorter than 50 amino acid residues and comprising at least 50% of SEQ ID NO:2.

In some embodiments, the fragment includes at least 60%, 70%, 80% or 90% of SEQ ID NO:2. In some embodiments, the fragment includes the entire SEQ ID NO:2. In some embodiments, the fragment consists of SEQ ID NO:2.

In some embodiments, the fragment is conjugated to a carrier protein, such as KLH (keyhole lympet hemocyanin), BSA (bovine serum albumin), OVA (ovalbumin), and THY (thyroglobulin).

### Preparation of Fresh Tumor Tissues for Immunostaining

The present disclosure also provides compositions and methods for processing tissue, such as fresh human or animal tissues, for immunohistochemical studies. The disclosed tissue preparation method is distinct from known methods of immunohistochemistry for human tissues. The present technology can preserve the tissue morphology and not mask antibody epitopes by chemical crosslinking using standard fixatives such as paraformaldehyde or formaldehyde.

Tissue, such as fresh frozen human tissues, are fixed according to certain embodiments of the present disclosure, following cryostat sectioning. The fixation can be done with non-crosslinking agents such as 100% methanol or a mixture of ethanol and acetic acid, and undergoes a period of, e.g., at least 5 hours and preferably overnight, incubation on glass slides. This technology is shown to be far superior to standard tissue preparation and fixation methods for immunohistochemistry of native tissues. It is also discovered that a longer fixation time (e.g., 5 hours or longer versus 30 minutes) can greatly increase the sensitivity of the subsequent immunostaining (see, e.g., FIG. 2). The longer fixation likely helps to dissociate the transmembrane protein from its associated proteins in the tissue sample so that the epitopes are more exposed during immunostaining. This method, without limitation, applies to both DAB (3,3′-diaminobenzidine) staining and immunofluorescence staining.

In some embodiments, the present disclosure provides a method for preparing a tissue sample for detection of the expression of a transmembrane protein in the tissue sample. The method may entail sectioning a tissue slide from the tissue sample, and fixing the tissue slide in a non-crosslinking fixative at a temperature of about 0° C. to 25° C.

The tissue sample may be a sample isolated from any tissue in a subject, such as a human subject. For instance, the tissue may be a blood sample, or a tissue block from the liver, bladder, colon, rectum, pancreas, lung, breast, urethra, head, neck, stomach, intestine, esophagus, ovary, kidney, skin, prostate, or thyroid. The subject may be one that is suffering from a disease such as diabetes, diabetic retinopathy, or cancer. In some embodiments, the subject is a human subject suffering from a cancer selected from bladder cancer, liver cancer, colon cancer, rectal cancer, endometrial cancer, leukemia, lymphoma, pancreatic cancer, small cell lung cancer, non-small cell lung cancer, breast cancer, urethral cancer, head and neck cancer, gastrointestinal cancer, stomach cancer, esophageal cancer, ovarian cancer, renal cancer, melanoma, prostate cancer and thyroid cancer.

The sample isolated, in some embodiments, may also include metastatic cells migrated from another tissue. For instance, metastatic cells from a colon tumor may migrate to the liver where the detection is made. As such, the present technology is also able to detect metastatic tumors.

Tissue sectioning can be done with methods known in the art. Generally, prior to sectioning, the tissue may be frozen with liquid nitrogen or in a freezer. The sectioning can be carried out on a Cryostat station. The slides may have a thickness from about 1 micron to about 20 microns, or from about 5 microns to about 16 microns.

A “non-crosslinking fixative” as used herein, refers to fixatives that do not form cross-linkage with molecules on the cells in a tissue sample. Crosslinking fixatives such as formaldehyde, paraformaldehyde, glutaraldehyde, acrolein, and osmium tetroxide primarily react with proteins or unsaturated lipids. A non-crosslinking fixative, by contrast, coagulates and/or precipitate proteins. For instance, alcohols such as methanol and ethanol coagulate and precipitate proteins in the tissue. Acids including acetic acid and picric acid mainly precipitate proteins. Additional examples of non-crosslinking fixatives include acetone and combinations of any non-crosslinking fixatives described herein.

In a preferred embodiments, the non-crosslinking fixative is methanol. In some embodiments, the tissue may be a blood sample, or a tissue block from the liver, bladder, colon, rectum, pancreas, lung, breast, urethra, head, neck, stomach, intestine, esophagus, ovary, kidney, skin, prostate, or thyroid. In some embodiments, the tissue includes blood vessels.

In some embodiments, the non-crosslinking fixative is ethanol or acetic acid, or a mixture thereof. In some embodiments, the tissue may be a blood sample, or a tissue block from the liver, bladder, colon, rectum, pancreas, lung, breast, urethra, head, neck, stomach, intestine, esophagus, ovary, kidney, skin, prostate, or thyroid. In some embodiments, the tissue may be a pancreatic tissue. In some embodiments, the tissue includes blood vessels.

In some embodiments, the non-crosslinking fixative is used at a concentration that is at least 90%, or at least 95%, 98%, 99%, 99.5%, 99.9%, or 99.99% or is about 100% (v/v). In some embodiments, the non-crosslinking fixative includes two or more agents, such as ethanol and acetic acid which may, collectively, have a concentration that is at least 90%, or at least 95%, 98%, 99%, 99.5%, 99.9%, or 99.99% or is about 100% (v/v). In some embodiments, when ethanol and acetic acid are mixed, their ratio is at least about 1:1 (v/v), or at least about 1.1:1, 1.2:1, 1.3:1, 1.4:1, 1.5:1, 1.6:1, 1.7:1, 1.8:1, 1.9:1, 2:1, 2.1:1, 2.2:1, 2.3:1, 2.4:1, 2.5:1, 2.6:1, 2.7:1, 2.8:1, 2.9:1 or 3:1 (v/v). In some embodiments, the ratio is not greater than about 6:1, 5.5:1, 5:1, 4.9:1, 4.8:1, 4.7:1, 4.6:1, 4.5:1, 4.4:1, 4.3:1, 4.2:1, 4.1:1, 4:1, 3.9:1, 3.8:1, 3.7:1, 3.6:1, 3.5:1, 3.4:1, 3.3:1, 3.2:1, 3.1:1 or 3:1 (v/v). In some embodiments, the ratio is from about 1:1 to about 5:1, from about 1.5:1 to about 4.5:1, from about 2:1 to about 4:1, from about 2.5:1 to about 3.5:1, from about 2.8:1 to about 3.2:1, from about 2.9:1 to about 3.1:1, or at about 3:1 (v/v).

The time period for the fixation varies depending on the need and on the type of the transmembrane protein. It is discovered herein that relatively longer time of fixation may results in more sufficient exposure of the epitope on the transmembrane protein. In certain embodiments, the fixation is carried out for 30 min to about 24 hours. In some embodiments, the fixation is carried out for at least 1 hour, 2 hours, 3 hours, 4 hours, 5 hours, 6 hours, 7 hours, 8 hours, 9 hours, 10 hours, 12 hours, 15 hours, 18 hours, 20 hours or 22 hours. In some embodiments, the fixation is carried out for no more than 6 hours, 7 hours, 8 hours, 9 hours, 10 hours, 12 hours, 15 hours, 18 hours, 20 hours or 24 hours.

The operating temperature for the fixation is generally between 0° C. to 25° C., but often at a temperature within 0-10° C., more preferably within 2-8° C., or around 4° C.

In some embodiments, prior to fixation, the tissue slide may need to be dried following sectioning. In some embodiments, the fixation starts relatively shortly following sectioning, such as within 24 hours, within 16 hours, within 12 hours, within 8 hours, within 4 hours, within 2 hours, within an hour, or within 30 minutes. In some embodiments, the fixation starts relatively shortly following drying, such as within 16 hours, within 12 hours, within 8 hours, within 4 hours, within 2 hours, within an hour, within 30 minutes, within 20 minutes, within 25 minutes, within 20 minutes, within 15 minutes, within 10 minutes or within 5 minutes.

In some embodiments, the tissue preparation method does not include treatment with any crosslinking fixative such as paraformaldehyde, formaldehyde, glutaraldehyde acrolein, and osmium tetroxide.

In some embodiments, the tissue sample was frozen within two hours after isolation from a human patient, to keep it fresh. In some embodiments, the tissue sample comprises a blood vessel, where the transmembrane protein is likely present.

Once the tissue slide is prepared, it can be used for detecting the transmembrane protein with immunohistochemical staining. The methods disclosed herein can be used to prepare tissue slides for immunohistochemical study of various transmembrane proteins. In some embodiments, the transmembrane protein is a cell surface tumor antigen. In some embodiments, the transmembrane protein is plexin domain containing 1 (PLXDC1) or plexin domain containing 2 (PLXDC2).

For detection or qualification of PLXDC1, the immunohistochemical staining can use an antibody that recognizes at least an amino acid residue of the PLXDC1 protein within SEQ ID NO:1 (SPQPGAGHDEGPGSGWAAKGTVRG). For detection or qualification of PLXDC2, the immunohistochemical staining can use an antibody that recognizes at least an amino acid residue of the PLXDC2 protein within SEQ ID NO:2 (KPGDQILDWQYGVTQAFPHTE). In some embodiments, the antibody is one or more disclosed in the present disclosure.

### Diagnostic Assays

The present invention provides, in part, methods, systems, and code for accurately classifying whether a biological sample expresses PLXDC1 or PLXDC2 and/or whether the levels of PLXDC1 or PLXDC2 are modulated (e.g., upregulated or downregulated), thereby indicative of the state of a disorder of interest, such as cancer. In some embodiments, the present invention is useful for classifying a sample (e.g., from a subject) as associated with or at risk for cancer or a subtype thereof, mediated by PLXDC1 or PLXDC2 using a statistical algorithm and/or empirical data (e.g., the presence, absence, or level of PLXDC1 or PLXDC2).

An exemplary method for detecting the level of PLXDC1, PLXDC2, or fragments thereof, and thus useful for classifying whether a sample is associated with a disease or disorder mediated by an aberrant expression (e.g., upregulation or downregulation) of PLXDC1, PLXDC2, or a clinical subtype thereof involves obtaining a biological sample from a test subject and contacting the biological sample with an antibody or antigen-binding fragment thereof of the present invention capable of detecting PLXDC1 or PLXDC2 such that the level of PLXDC1 or PLXDC2 is detected in the biological sample. In some embodiments, at least one antibody or antigen-binding fragment thereof is used, wherein two, three, four, five, six, seven, eight, nine, ten, or more such antibodies or antibody fragments can be used in combination (e.g., in sandwich ELISAs) or in serial. In certain instances, the statistical algorithm is a single learning statistical classifier system. For example, a single learning statistical classifier system can be used to classify a sample as a PLXDC1 or PLXDC2 sample based upon a prediction or probability value and the presence or level of PLXDC1 or PLXDC2, respectively The use of a single learning statistical classifier system typically classifies the sample as a PLXDC1 or PLXDC2 sample with a sensitivity, specificity, positive predictive value, negative predictive value, and/or overall accuracy of at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%.

In other embodiments, the method of the present invention further provides a diagnosis in the form of a probability that the individual has a condition or disorder associated with PLXDC1 or PLXDC2. For example, the individual can have about a 0%, 5%, 10%, 15%, 20%, 25%, 30%, 35%, 40%, 45%, 50%, 55%, 60%, 65%, 70%, 75%, 80%, 85%, 90%, 95%, or greater probability of having the condition or disorder. In yet other embodiments, the method of the present invention further provides a prognosis of the condition or disorder in the individual. In some instances, the method of classifying a sample as a PLXDC1 or PLXDC2 sample is further based on the symptoms (e.g., clinical factors) of the individual from which the sample is obtained. The symptoms or group of symptoms can be, for example, lymphocyte count, white cell count, erythrocyte sedimentation rate, diarrhea, abdominal pain, cramping, fever, anemia, weight loss, anxiety, depression, and combinations thereof. In some embodiments, the diagnosis of an individual as having a condition or disorder associated with PLXDC1 or PLXDC2 is followed by administering to the individual a therapeutically effective amount of a drug useful for treating one or more symptoms associated with the condition or disorder (e.g., chemotherapeutic agents).

In some embodiments, the methods further involve obtaining a control biological sample (e.g., biological sample from a subject who does not have a condition or disorder mediated by PLXDC1 or PLXDC2), a biological sample from the subject during remission or before developing a condition or disorder mediated by PLXDC1 or PLXDC2, or a biological sample from the subject during treatment for developing a condition or disorder mediated by PLXDC1 or PLXDC2.

An exemplary method for detecting the presence or absence of PLXDC1 or PLXDC2 polypeptide or fragments thereof is an antibody of the present invention, or fragment thereof, capable of binding to a PLXDC1 or PLXDC2 polypeptide, preferably an antibody with a detectable label. Antibodies can be polyclonal, or more preferably, monoclonal. Such agents can be labeled. The term “labeled”, with regard to the antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody. The term “biological sample” is intended to include tissues, cells, and biological fluids isolated from a subject, such as serum, as well as tissues, cells, and fluids present within a subject. That is, the detection method of the present invention can be used to detect PLXDC1 or PLXDC2, or fragments thereof, in a biological sample in vitro as well as in vivo. In vitro techniques for detection of PLXDC1 or PLXDC2 polypeptide include enzyme linked immunosorbent assays (ELISAs), Western blots, immunoprecipitations, immunohistochemistry (RIC), intracellular flow cytometry and related techniques, and immunofluorescence. Furthermore, in vivo techniques for detection of a PLXDC1 or PLXDC2 polypeptide or a fragment thereof include introducing into a subject a labeled anti-PLXDC1 or anti-PLXDC2 antibody. For example, the antibody can be labeled with a radioactive, luminescent, fluorescent, or other similar marker whose presence and location in a subject can be detected by standard imaging techniques, either alone or in combination with imaging for other molecules, such as markers of cell type.

In certain embodiments, the biological sample contains polypeptide molecules from the test subject. Preferred biological samples are serum, tumor microenvironment, peritumoral, or intratumoral, e.g., isolated by conventional means, from a subject.

In other embodiments, the methods further involve obtaining a control biological sample from a control subject, contacting the control sample with a compound or agent capable of detecting PLXDC1 or PLXDC2 polypeptide, or fragments thereof, such that the presence of PLXDC1 or PLXDC2 polypeptide, or fragments thereof, is detected in the biological sample, and comparing the presence of PLXDC1 or PLXDC2 polypeptide, or fragments thereof, in the control sample with the presence of PLXDC1 or PLXDC2 polypeptide, or fragments thereof in the test sample.

In still other embodiments, the antibodies can be associated with a component or device for the use of the antibodies in an ELISA or RIA. Non-limiting examples include antibodies immobilized on solid surfaces for use in these assays (e.g., linked and/or conjugated to a detectable label based on light or radiation emission as described above). In other embodiments, the antibodies are associated with a device or strip for detection of PLXDC1 or PLXDC2 by use of an immunochromatographic or immunochemical assay, such as in a “sandwich” or competitive assay, immunohistochemistry, immunofluorescence microscopy, and the like. Additional examples of such devices or strips are those designed for home testing or rapid point of care testing. Further examples include those that are designed for the simultaneous analysis of multiple analytes in a single sample. For example, an unlabeled antibody of the invention may be applied to a “capture” PLXDC1 or PLXDC2 polypeptides in a biological sample and the captured (or immobilized) PLXDC1 or PLXDC2 polypeptides may be bound to a labeled form of an anti-PLXDC1 or anti-PLXDC2 antibody of the invention for detection. Other standard embodiments of immunoassays are well-known the skilled artisan, including assays based on, for example, immunodiffusion, immunoelectrophoresis, immunohistopathology, immunohistochemistry, and histopathology.

### Identification and Treatment of Cancer Patients

The present technology can be used to identify subjects that may be suffering from or at the risk of developing a disease or condition characterized with expression, under-expression, or over-expression of the target transmembrane protein. Once identified, the subject can be subjected to suitable treatment or other medical interventions. PLXDC1, for instance, may be expressed (or over-expressed) in the blood vessels of certain tumor types. A patient having detected for such an expression, therefore, may be suitable for a treatment with an agent that inhibits PLXDC1 or the PLXDC1 signaling pathway.

The expression of PLXDC2 in tumor samples, however, has not been well established. Neither is the role of PLXDC2 in tumorigenesis or tumor therapy. It is discovered herein that PLXDC2 was detected in tumor blood vessels in human liver cancer and other metastatic cancers with metastatic cells migrated to the liver.

In one aspect, therefore, provided is a method for identifying a human cancer patient suitable for an anti-PLXDC2 (plexin domain containing 2) therapy. The method entails detecting the expression of the PLXDC2 protein in a sample isolated from the patient, wherein expression of the PLXDC2 protein in the liver sample indicates that the patient is suitable for a therapy comprising an agent that inhibits the PLXDC2 signaling.

In some embodiments, the sample is a liver sample. In some embodiments, the agent is an anti-PLXDC2 antibody.

Also provided, in some embodiments, is a method for treating a human cancer patient identified as having expression of the PLXDC2 (plexin domain containing 2) protein in the liver, comprising administering to the patient an agent that inhibits the PLXDC2 signaling. The patient may be suffering from liver cancer or a metastatic cancer that has spread to liver. In some embodiments, the agent is an anti-PLXDC2 antibody.

### Therapeutic Compositions and Methods

The present disclosure is further directed to antibody-based therapies which involve administering the antibodies of the disclosure to a patient such as an animal, a mammal, and a human for treating one or more of the disorders or conditions described herein. Therapeutic compounds of the disclosure include, but are not limited to, antibodies of the disclosure (including variants and derivatives thereof as described herein) and nucleic acids or polynucleotides encoding antibodies of the disclosure (including variants and derivatives thereof as described herein).

Accordingly, in some embodiments, provided are methods for treating a cancer in a patient in need thereof. The method, in some embodiments, entails administering to the patient an effective amount of an antibody of the present disclosure.

By a “therapeutically effective amount” of the polypeptide of the invention is meant a sufficient amount of the antibody to treat the disorder of interest, such as cancer, at a reasonable benefit/risk ratio applicable to any medical treatment. It will be understood, however, that the total daily usage of the antibodies and compositions of the present invention will be decided by the attending physician within the scope of sound medical judgment. The specific therapeutically effective dose level for any particular patient will depend upon a variety of factors including the disorder being treated and the severity of the disorder; activity of the specific antibody employed; the specific composition employed, the age, body weight, general health, sex and diet of the patient; the time of administration, route of administration, and rate of excretion of the specific antibody employed; the duration of the treatment; drugs used in combination or coincidental with the specific polypeptide employed; and like factors well-known in the medical arts. For example, it is well-known within the skill of the art to start doses of the compound at levels lower than those required to achieve the desired therapeutic effect and to gradually increase the dosage until the desired effect is achieved.

Tumors, including tumors of the local tissue or tumor cells migrated from a metastatic tumor from another tissue, that express the PLXDC1 or PLXDC2 protein include those of bladder cancer, non-small cell lung cancer, renal cancer, breast cancer, urethral cancer, colorectal cancer, head and neck cancer, squamous cell cancer, Merkel cell carcinoma, gastrointestinal cancer, stomach cancer, esophageal cancer, ovarian cancer, renal cancer, and small cell lung cancer. Accordingly, the presently disclosed antibodies can be used for treating any one or more such cancers. In some embodiments, the cancer patient being treated has PLXDC1 or PLXDC2 expressed in the tumor endothelial cells or tumor cells.

The present disclosure also provides pharmaceutical compositions. Such compositions comprise an effective amount of an antibody, and an acceptable carrier. In some embodiments, the composition further includes a second anticancer agent (e.g., an immune checkpoint inhibitor).

In certain embodiments, the term “pharmaceutically acceptable” means approved by a regulatory agency of the Federal or a state government or listed in the U.S. Pharmacopeia or other generally recognized pharmacopeia for use in animals, and more particularly in humans. Further, a “pharmaceutically acceptable carrier” will generally be a non-toxic solid, semisolid or liquid filler, diluent, encapsulating material or formulation auxiliary of any type.

The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents such as acetates, citrates or phosphates. Antibacterial agents such as benzyl alcohol or methyl parabens; antioxidants such as ascorbic acid or sodium bisulfite; chelating agents such as ethylenediaminetetraacetic acid; and agents for the adjustment of tonicity such as sodium chloride or dextrose are also envisioned. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in Remington's Pharmaceutical Sciences by E. W. Martin, incorporated herein by reference. Such compositions will contain a therapeutically effective amount of the antigen-binding polypeptide, preferably in purified form, together with a suitable amount of carrier so as to provide the form for proper administration to the patient. The formulation should suit the mode of administration. The parental preparation can be enclosed in ampoules, disposable syringes or multiple dose vials made of glass or plastic.

In certain embodiments, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The pharmaceutical compositions can be included in a container, pack, or dispenser together with instructions for administration.

### EXAMPLES

The following examples are included to demonstrate specific embodiments of the disclosure. It should be appreciated by those of skill in the art that the techniques disclosed in the examples which follow represent techniques to function well in the practice of the disclosure, and thus can be considered to constitute specific modes for its practice. However, those of skill in the art should, in light of the present disclosure, appreciate that many changes can be made in the specific embodiments which are disclosed and still obtain a like or similar result without departing from the spirit and scope of the disclosure.

### Example 1: DAB Immunostaining Method

This example describes a procedure to prepare a tissue slide for DAB (3,3′-diaminobenzidine) immunostaining. The steps are as follow:
1. Freeze a fresh tumor (less than 2 hours old) immediately in optimum cutting temperature compound (OCT) using liquid nitrogen. The frozen block can be stored at −80° C. for up to two weeks.
2. Section the tumor block in −20° C. in a Cryostat in approximately 16 micron thickness. Dry the sections for 10-15 min and then immediately fix all sections in 100% methanol at 4° C. for 5-6 hours in plastic mailers. Once fixed by methanol, the slides can be stored at −20° C. or used immediately.
3. To start immunostaining, add up to 5 methanol-fixed slides per plastic mailer. Remove OCT by soaking slides in PBS+(PBS and 2 mM MgCl₂) for 10 min.
4. Wipe clean the edges to completely dry and draw around the slide using the PAP pen. After the PAP pen line dries for 10 min, add PBS+ to the section to rehydrate the sections.
5. Block the sections with Blocking Buffer containing horse serum from Vectastain ABC Kit for 1 hour at room temperature in a humidified chamber.
6. Start primary anti-hTEM7 or anti-PLXDC2 antibody diluted in the Blocking Buffer at room temperature for 2 hours or 4° C. overnight in a humidified chamber.
7. Block the sections using 0.5 mg/ml avidin in PBS+ for 30 min to block endogenous biotin in the tissue sections.
8. Wash 3× using PBS+ and block the sections using 2 mM biotin in PBS+ for 30 min.
9. Wash 3× using PBS+. Incubate the sections with 3% H₂O₂ freshly diluted in PBS for 5 min and wash 3× using PBS+ again
10. Wash the sections with PBS+ for 5-10 min 3 times. Incubate sections for 45 min with biotinylated secondary antibody diluted in Blocking Buffer. At the same time mix Vector reagent A and B in PBS for at least 30 min.
11. Wash the sections with PBS+ for 5-10 min 3 times.
12. Incubate sections for 30 min with Vector A+B mix diluted 2× in 1 mg/ml BSA in PBS+.
13. Wash the sections with PBS+ for 5-10 min 3 times.
14. Incubate each section with 150-200 μl of freshly prepared ImmPACT DAB substrate
15. After 1-5 min of color development, stop the staining by washing with PBS+ twice.
16. Spread VectaMount onto tissue sections while the sections are still wet with xylene. Apply the coverslip and dry for overnight.

### Example 2: Immunofluorescence Staining Method

This example describes a procedure to prepare a tissue slide for immunofluorescence staining. The steps are as follow:
1. Freeze a fresh tumor (less than 2 hours old) immediately in OCT using liquid nitrogen. The frozen block can be stored at −80° C. for up to two weeks.
2. Section the tumor block in −20° C. in a Cryostat in approximately 16 micron thickness. Dry the sections for 10-15 min and then immediately fix all sections in methanol at 4° C. for 2-3 hours in plastic mailers. Once fixed, the slides can be stored at −20° C. or used immediately (more ideal).
3. To start immunostaining, add up to 5 methanol-fixed slides per plastic mailer. Remove OCT by soaking slides in PBS+ for 5 min.
4. Take each slide out of the mailer. Wipe clean the edges to completely dry and draw around the edges of the slide using the PAP pen.
5. After the PAP pen line dries for 10 min, add PBS+ to the section to rehydrate the sections.
6. Block the sections with Blocking Buffer (5% Normal goat serum, 0.3% Triton in PBS+) 1 hour at room temperature a humidified chamber.
7. Start primary anti-hTEM7 or anti-PLXDC2 antibody in the Blocking Buffer at room temperature for 2 hours or 4° C. overnight in a humidified chamber. It is important to include an adjacent section without adding the primary antibody to control for autofluorescence in human tissues.
8. Wash the sections with PBS+ for 10 min 3 times. Incubate sections for one hour using fluorescently labeled goat anti-rabbit secondary antibody diluted in the Blocking Buffer.
9. Wash the sections with PBS+ for 5-10 min 3 times and mount the sections using aqueous mounting media.

### Example 3: Immunostaining with Anti-PLXDC1 and Anti-PLXDC2 Antibodies

The above methods were used to generate data as shown in FIGS. 1-5. Rabit polyclonal antibodies were raised specifically to epitopes close to the N-terminus of each of TEM7 (PLXDC1) and PLXDC2. These epitopes, SEQ ID NO:1 and 2 (see Table 1) were selected in the least conserved regions of PLXDC1 and PLXDC2 such that the antibodies could distinguish the two receptor proteins.

In FIGS. 1A-1D, the polyclonal antibodies specifically recognized human TEM7 (PLXDC1) and PLXDC2 in immunostaining. FIG. 1A shows that polyclonal antibody against human TEM7, Anti-hTEM7 Antibody, recognized human TEM7 transfected into HEK293 cells. FIG. 1B shows that the polyclonal antibody against human TEM7 did not recognize human PLXDC2 transfected into HEK293 cells or untransfected cells. FIG. 1C shows that polyclonal antibody against human PLXDC2, Anti-hPLXDC2 Antibody, did not recognize human TEM7 transfected into HEK293 cells or untransfected cells. FIG. 1D shows that the polyclonal antibody against human PLXDC2 recognized human PLXDC2 transfected into HEK293 cells. In FIGS. 1A-D, antibody staining signal is in green and cell nuclei are in blue. Cells were fixed with 100% methanol.

FIGS. 2A-2D show that longer fixation using 100% methanol is important in revealing TEM7 immunostaining signals in the human tumor samples. Five hours of methanol fixation (FIG. 2A) revealed more robust TEM7 signals in tumor blood vessels than two hours of methanol fixation (FIG. 2B) of fresh frozen tumor sections. TEM7 was detected using the polyclonal antibody against TEM7. As a control, the tumor sections were stained using antibody against VEGFR2, a marker of blood vessels (FIG. 2C). A control staining by omitting the primary antibody is shown in FIG. 2D. All sections are from the same human liver cancer tumor (hepatocellular carcinoma). Immunostaining signal is in brown color in FIGS. 2A-D.

FIGS. 3A-3F show the results of immunostaining of human liver cancer tumor (hepatocellular carcinoma) by polyclonal antibodies against human TEM7. FIGS. 3A and 3B show that polyclonal antibody against human TEM7 recognized human TEM7 expressed in tumor blood vessels in human liver cancer. FIGS. 3C and 3D show control immunostaining using antibody against VEGFR2, a known marker of blood vessels in different sections of the same tumor. FIGS. 3E and 3F show control immunostaining without the primary antibody (but with all other steps). Antibody staining signal is in brown color in FIGS. 3A-3F. This liver cancer tumor is from a different cancer patient as the tumors in FIGS. 2A-2D.

Immunostaining of human liver cancer tumor (hepatocellular carcinoma) by polyclonal antibodies against human TEM7 (PLXDC1) and PLXDC2 is shown in FIGS. 4A-4F. FIGS. 4A-4C show staining in the region of the tumor that has abundant large tumor vessels. FIGS. 4D-4F show staining in the region of the tumor that has mostly tumor microvessels. FIGS. 4A and 4D show that polyclonal antibody against human TEM7 recognizes human TEM7 expressed in tumor blood vessels in human liver cancer. FIGS. 4B and 4E show that polyclonal antibody against human PLXDC2 recognized human PLXDC2 expressed in tumor blood vessels in human liver cancer. Like PLXDC1, PLXDC2 was also highly enriched in tumor blood vessels in this liver cancer tumor. FIGS. 4C and 4F show control immunostaining using antibody against VEGFR2, a known marker of blood vessels in different sections of the same tumor. Antibody staining signal is in brown color in FIGS. 4A-4F. This liver cancer tumor is from a different cancer patient as the tumors in FIGS. 2A-2D and FIGS. 3A-3F.

Immunostaining of a metastatic human tumor from colon cancer by polyclonal antibodies against human TEM7 (PLXDC1) and PLXDC2 is shown in FIGS. 5A-5B. FIG. 5A shows that polyclonal antibody against human TEM7 recognized human TEM7 expressed in tumor blood vessels in human metastatic colon cancer. FIG. 5B shows that polyclonal antibody against human PLXDC2 recognized human PLXDC2 expressed in tumor blood vessels in human metastatic colon cancer Like PLXDC1, PLXDC2 was also highly enriched in tumor blood vessels in this metastatic colon cancer tumor. Antibody staining signal is in brown color in FIGS. 5A-5B.

### Example 4: Sample Preparation and Immunostaining of Pancreatic Tumor Tissues

The sample preparation and immunostaining methods described in Examples 1 and 2 were applied on tumor tissues from a patient with pancreatic tumor. In addition to the use of 100% methanol for fixation, this example also tested an ethanol/acetic acid (75%/25%, v/v) mixture. The same polyclonal antibodies for TEM7 (PLXDC1) and PLXDC2 were used for immunostaining.

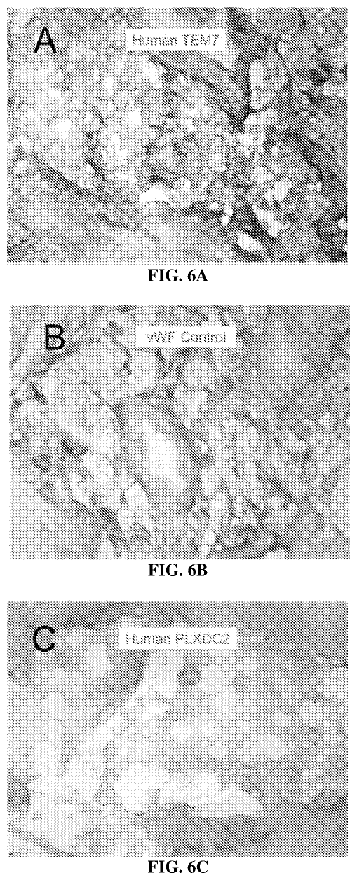

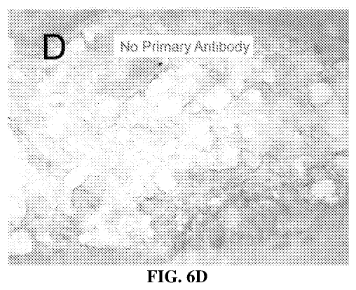

The immunostaining results for TEM7 in ethanol/acetic acid-fixed pancreatic tumor tissues are presented in FIGS. 6A-6B. In FIG. 6A, polyclonal antibody against human TEM7 recognized human TEM7 expressed in tumor blood vessels of a pancreatic tumor tissue. As a positive control an antibody against von Willebrand factor (vWF), a general marker of blood vessels, demonstrates the effectiveness of the fixation method (FIG. 6B). Unlike TEM7, PLXDC2 was not expressed in these samples (FIG. 6C). FIG. 6D presents control staining without a primary antibody. It is interesting to note that the immunostaining of these ethanol/acetic acid-fixed pancreatic tumor tissues showed even more robust signals than those fixed with methanol.

This example also demonstrates that TEM7 (PLXDC1) is expressed in the blood vessels of pancreatic tumor tissues. Accordingly, pancreatic tumors that express TEM7 may be suitable for therapies that target the TEM7 protein.

Unless otherwise defined, all technical and scientific terms used herein have the same meaning as commonly understood by one of ordinary skill in the art to which this invention belongs.

The inventions illustratively described herein may suitably be practiced in the absence of any element or elements, limitation or limitations, not specifically disclosed herein. Thus, for example, the terms “comprising”, “including,” “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed.

Thus, it should be understood that although the present invention has been specifically disclosed by preferred embodiments and optional features, modification, improvement and variation of the inventions embodied therein herein disclosed may be resorted to by those skilled in the art, and that such modifications, improvements and variations are considered to be within the scope of this invention. The materials, methods, and examples provided here are representative of preferred embodiments, are exemplary, and are not intended as limitations on the scope of the invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

In addition, where features or aspects of the invention are described in terms of Markush groups, those skilled in the art will recognize that the invention is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

It is to be understood that while the disclosure has been described in conjunction with the above embodiments, that the foregoing description and examples are intended to illustrate and not limit the scope of the disclosure. Other aspects, advantages and modifications within the scope of the disclosure will be apparent to those skilled in the art to which the disclosure pertains.

1. A method for preparing a tissue sample for detection of the expression of a transmembrane protein in the tissue sample, comprising:
sectioning a tissue slide from the tissue sample, and
fixing the tissue slide in a non-crosslinking fixative at a temperature of about 0° C. to 25° C.

2. The method of claim 1, wherein the non-crosslinking fixative is selected from the group consisting of methanol, ethanol, acetone, acetic acid and combinations thereof.

3. The method of claim 1, wherein the non-crosslinking fixative is methanol.

4. The method of claim 1, wherein the non-crosslinking fixative is used at a concentration that is at least 99%.

5. The method of claim 2, wherein the non-crosslinking fixative comprises ethanol and acetic acid.

6. The method of claim 5, wherein the non-crosslinking fixative comprises ethanol and acetic acid at a ratio of about 2:1 to about 4:1 (v/v).

7. The method of claim 1, wherein the tissue slide is fixed in the non-crosslinking fixative for about 2 hours to about 24 hours.

8. The method of claim 7, wherein the tissue slide is fixed in the non-crosslinking fixative for 5 to 16 hours.

9. The method of claim 1, further comprising drying the tissue slide prior to fixing.

10. The method of any one of claims 1-9, wherein the fixing starts within 16 hours following the sectioning.

11. The method of claim 10, wherein the fixing starts within 2 hours following the sectioning.

12. The method of claim 10, wherein the fixing starts within 30 minutes following the drying.

13. The method of any one of claims 1-12, wherein the tissue block and tissue slide are not treated with a crosslinking fixative.

14. The method of claim 13, wherein the crosslinking fixative comprises paraformaldehyde, formaldehyde, glutaraldehyde acrolein, and osmium tetroxide.

15. The method of any one of claims 1-14, wherein the transmembrane protein is Plexin domain containing 1 (PLXDC1) or Plexin domain containing 2 (PLXDC2).

16. The method of claim 15, further comprising detecting the transmembrane protein with immunohistochemical staining of the tissue slide.

17. The method of claim 16, wherein the immunohistochemical staining uses an antibody that recognizes at least an amino acid residue of the PLXDC1 protein within SEQ ID NO:1 (SPQPGAGHDEGPGSGWAAKGTVRG).

18. The method of claim 16, wherein the immunohistochemical staining uses an antibody that recognizes at least an amino acid residue of the PLXDC2 protein within SEQ ID NO:2 (KPGDQILDWQYGVTQAFPHTE).

19. The method of any one of claims 1-18, wherein the tissue sample was frozen within two hours after isolation from a human patient.

20. The method of claim 19, wherein the tissue sample comprises a blood vessel.

21. The method of any one of claims 19-20, wherein the human patient suffers from tumor or diabetic retinopathy.

22. The method of any one of claims 1-21, wherein the tissue slide has a thickness of about 1 micrometer to about 25 micrometers.

23. An antibody or antigen-binding fragment thereof having binding specificity to a human Plexin domain containing 1 (PLXDC1) protein, wherein the antibody or fragment thereof is capable of binding to at least one of the amino acid residues within SEQ ID NO:1 (SPQPGAGHDEGPGSGWAAKGTVRG).

24. The antibody or fragment thereof of claim 23, which is capable of binding to at least two non-adjacent amino acid residues within SEQ ID NO:1.

25. The antibody or fragment thereof of claim 23 or 24, wherein the binding between the antibody or fragment thereof and amino acid residues in SEQ ID NO:1 is sufficient to maintain the binding specificity between the antibody or fragment thereof and the PLXDC1 protein.

26. The antibody or fragment thereof of any one of claims 23-25, which is a polyclonal antibody or fragment thereof.

27. The antibody or fragment thereof of any one of claims 23-25, which is a monoclonal antibody or fragment thereof.

28. The antibody or fragment thereof of claim 23, which is obtained by immunizing an animal with a fusion protein comprising a fragment of PLXDC1 shorter than 50 amino acid residues and comprising at least 50% of SEQ ID NO:1.

29. The antibody or fragment thereof of claim 26, wherein the fragment comprises SEQ ID NO:1.

30. An antibody or antigen-binding fragment thereof having binding specificity to a human Plexin domain containing 2 (PLXDC2) protein, wherein the antibody or fragment thereof is capable of binding to at least one of the amino acid residues within SEQ ID NO:2 (KPGDQILDWQYGVTQAFPHTE).

31. The antibody or fragment thereof of claim 30, which is capable of binding to at least two non-adjacent amino acid residues within SEQ ID NO:2.

32. The antibody or fragment thereof of claim 30 or 31, wherein the binding between the antibody or fragment thereof and amino acid residues in SEQ ID NO:2 is sufficient to maintain the binding specificity between the antibody or fragment thereof and the PLXDC2 protein.

33. The antibody or fragment thereof of any one of claims 30-32, which is a polyclonal antibody or fragment thereof.

34. The antibody or fragment thereof of any one of claims 30-32, which is a monoclonal antibody or fragment thereof.

35. The antibody or fragment thereof of claim 30, which is obtained by immunizing an animal with a fusion protein comprising a fragment of PLXDC2 shorter than 50 amino acid residues and comprising at least 50% of SEQ ID NO:2.

36. The antibody or fragment thereof of claim 35, wherein the fragment comprises SEQ ID NO:1.

37. A method of detecting the expression of PLXDC1 or PLXDC2 in a human sample, comprising contacting the sample with an antibody or fragment thereof of any one of claims 23-36, and detecting the binding of the antibody to the PLXDC1 or PLXDC2 in the sample.

38. The method of claim 37, wherein the antibody or fragment thereof is radiolabeled.

39. The method of claim 38, wherein the antibody or fragment thereof is labeled with a positron-emitting radionuclide.

40. The method of claim 39, wherein the positron-emitting radionuclide is selected from the group consisting of ¹⁸F, ¹²⁴I, ⁸⁹Zr, ⁶⁸Ga, ⁶⁴Cu, and ⁷⁶Br.

41. The method of any one of claims 37-40, wherein the contacting is ex vivo.

42. The method of any one of claims 37-40, wherein the contacting is in vivo.

43. The method of claim 39 or claim 42, wherein the detection is carried out with positron emission tomography (PET).

44. A method of treating a cancer patient that has PLXDC1 or PLXDC2 expressed in tumor endothelial cells or tumor cells, comprising administering to the patient an antibody or fragment thereof of any one of claims 23-36.

45. A method for identifying a human cancer patient suitable for an anti-PLXDC2 (Plexin domain containing 2) therapy, comprising detecting the expression of the PLXDC2 protein in a liver cancer tumor sample isolated from the patient, wherein expression of the PLXDC2 protein in the liver sample indicates that the patient is suitable for a therapy comprising an agent that inhibits the PLXDC2 signaling.

46. The method of claim 45, wherein the agent is an anti-PLXDC2 antibody.

47. A method for treating a human cancer patient identified as having expression of the PLXDC2 (Plexin domain containing 2) protein in the liver, comprising administering to the patient an agent that inhibits the PLXDC2 signaling.

48. The method of claim 47, wherein the patient suffers from liver cancer or a metastatic cancer that has spread to liver.

49. The method of claim 48, wherein the agent is an anti-PLXDC2 antibody.
